(19) United States
(12) Patent Application Publication
(10) Pub. No.: US 2021/0222121 A1
(43) Pub. Date: Jul. 22, 2021

(54) COMPOSITIONS AND METHODS FOR ENHANCING SPERM FUNCTION

(71) Applicant: OHANA BIOSCIENCES, INC., Cambridge, MA (US)

(72) Inventors: Kathleen Inez Seyb, Wakefield, MA (US); Felipe A. Navarrete Solano, Medford, MA (US); Eric Steven Furfine, Lincoln, MA (US); Christopher R. Perley, Tewksbury, MA (US); Nicolas Da Silva Santos, Cambridge, MA (US)

(21) Appl. No.: 17/221,683

(22) Filed: Apr. 2, 2021

Related U.S. Application Data
(63) Continuation of application No. PCT/US2019/063687, filed on Nov. 27, 2019.
Application No. PCT/US2019/063687 is a continuation-in-part of application No. 16/282,224, filed on Feb. 21, 2019, now abandoned.
Application No. 16/282,224 is a continuation-in-part of application No. 16/282,204, filed on Feb. 21, 2019, now Pat. No. 10,603,075.
Application No. 16/282,204 is a continuation-in-part of application No. 16/282,217, filed on Feb. 21, 2019, now Pat. No. 10,470,798.
(60) Provisional application No. 62/914,803, filed on Oct. 14, 2019.
(60) Provisional application No. 62/773,448, filed on Nov. 30, 2018.
(60) Provisional application No. 62/773,453, filed on Nov. 30, 2018.
(60) Provisional application No. 62/773,462, filed on Nov. 30, 2018.
(60) Provisional application No. 62/773,471, filed on Nov. 30, 2018.
(60) Provisional application No. 62/773,440, filed on Nov. 30, 2018.
(60) Provisional application No. 62/773,433, filed on Nov. 30, 2018.

(51) Int. Cl. C12N 5/076; C12N 15/873

(52) U.S. Cl. CPC C12N 5/061; C12N 15/873; C12N 2517/10; A61B 17/43; C12N 2500/12; C12N 2500/60; C12N 2500/34; C12N 2500/38

(57) ABSTRACT

The disclosure provides, inter alia, methods of improving sperm function and related methods of fertilization, together with preparations of activated or potentiated sperm. The methods provided by the disclosure, in some embodiments entail energy depletion with subsequent staged reintroduction of different energy sources. The disclosure additionally provides articles of manufacture suitable for performing the methods provided by the invention. The invention provides kits for separating sperm and for processing and preparing sperm for, in some embodiments, IVF or IUI. Also provided are nutrient free reagents useful preparing sperm.

### CROSS-REFERENCE

This application claims priority to International Application No. PCT/US2019/063687, filed Nov. 27, 2019, which claims the benefit of U.S. Provisional Application No. 62/773,448, filed Nov. 30, 2018, U.S. Provisional Application No. 62/773,453, filed Nov. 30, 2018, U.S. Provisional Application No. 62/773,462, filed Nov. 30, 2018, U.S. Provisional Application No. 62/773,471, filed Nov. 30, 2018, U.S. Provisional Application No. 62/773,433, filed Nov. 30, 2018, U.S. Provisional Application No. 62/773,440, filed Nov. 30, 2018, U.S. Provisional Application No. 62/914,803, filed Oct. 14, 2019, U.S. patent application Ser. No. 16/282,204, filed Feb. 21, 2019, U.S. patent application Ser. No. 16/282,217, filed Feb. 21, 2019, and U.S. patent application Ser. No. 16/282,224, filed Feb. 21, 2019, each of which application is incorporated herein by reference in its entirety.

### BACKGROUND

Male factor is a contributing factor for ˜50% of couples having difficulty conceiving. An important aspect of assisted reproduction is obtaining maximal function of male gametes (sperm) to help maximize fertilization. Accordingly, a need exists for media, compositions, and methods for increasing sperm function, e.g., to facilitate assisted reproduction.

### SUMMARY

The invention provides, inter alia, media, compositions, and methods for increasing sperm function, e.g., to facilitate assisted reproduction.

Provided herein are methods for promoting fertilization comprising: (a) incubating a mammalian sperm under energy depletion conditions for a time suitable to potentiate the mammalian sperm, (b) providing the potentiated mammalian sperm from step (a) with an effective amount of a first energy source and a second energy source in a serial manner, and (c) providing the mammalian sperm resulting from step (b) with access to an egg under conditions to promote fertilization, wherein the effective amount is an amount sufficient to induce improved sperm function.

Provided herein are methods of inducing increased sperm function comprising: (a) incubating a mammalian sperm under energy depletion conditions for a time suitable to generate a potentiated mammalian sperm, (b) providing the potentiated mammalian sperm from step (a) with an effective amount of a first energy source selected from: (i) a glycolytic energy source or (ii) a gluconeogenesis substrate, and (c) subsequently providing the mammalian sperm from step (b) with an effective amount of a second energy source, selected from: (i) the glycolytic energy source or (ii) the gluconeogenesis substrate, wherein the second energy source provided is not selected in step (b), wherein the effective amount is an amount sufficient to induce increased sperm function.

Provided herein are preparations of sperm comprising increased percentage of hyperactivated sperm prepared by a process of: (a) incubating a mammalian sperm under energy depletion conditions for a time suitable to generate a potentiated mammalian sperm, and (b) providing the potentiated mammalian sperm from step (a) with an effective amount of a first energy source and a second energy source in a serial manner, wherein the effective amount is an amount sufficient to induce an increase in one or more sperm function, and wherein the preparation of sperm comprising increased percentage of hyperactivated sperm comprises an increase in one or more sperm function relative to suitable control sperm selected from: an untreated mammalian sperm, the potentiated mammalian sperm of step (a) provided with an effective amount of the first energy source or the second energy source independently, the potentiated mammalian sperm of step (a) provided with an effective amount of the first energy source and the second energy source simultaneously, or a mammalian sperm treated with standard capacitation medium, such as C-HTF.

Provided herein are methods of inducing increased sperm function comprising; (a) providing a mammalian sperm in a preservation medium comprising a buffer and having a pH of between about: 6-7 and an osmolality of between about: 300 and 400 mOsm/kg, (b) incubating the mammalian sperm under energy depletion conditions for a time suitable to potentiate the mammalian sperm, and (c) providing the potentiated mammalian sperm from step (b) with an effective amount of (i) a glycolytic energy source and/or (ii) a gluconeogenesis substrate, thereby inducing increased sperm function compared to a suitable control sperm.

Provided herein are methods for promoting fertilization comprising: (a) incubating a mammalian sperm under energy depletion conditions for a time suitable to potentiate the sperm, wherein prior to the incubating, the mammalian sperm is stored in a preservation medium comprising a buffer and having a slightly acidic pH and an osmolality of between about: 300 and 400 mOsm/kg, (b) providing the potentiated sperm from step (a) with an effective amount of a first energy source and a second energy source in a serial manner, (c) increasing one or more of sperm function, to a level greater than that obtained by providing the potentiated mammalian sperm of step (a) with an effective amount of the first energy source or the second energy source independently, or providing an effective amount of the first energy source and the second energy source simultaneously; and

(d) providing the mammalian sperm with increased function with access to an egg under conditions to promote fertilization.

Provided herein are kits comprising: a) a first container comprising a first composition comprising a buffered sperm-potentiating energy depletion composition, and b) a second container comprising a second composition comprising at least a first energy source suitable for a mammalian sperm,

wherein, upon incubating the mammalian sperm in the first composition for a suitable time, generates a potentiated mammalian sperm, and wherein, upon providing the potentiated mammalian sperm an effective amount of at least the first energy source, increases function of the potentiated mammalian sperm relative to a suitable control.

Provided herein are preparations of hyperactivated sperm comprising at least 5% hyperactivated sperm, optionally wherein the preparation has not been previously sorted on the basis of hyperactivation, optionally wherein the hyperactivated and/or intermediate sperm have 10, 15, 20, 25, 30, 35, 40, 45, 50%, or more (e.g., 2, 3, 4, 5, 6, 7, 8, 9, 10-fold, or more) reduction in intracellular RNA concentration (such as small non-coding RNA, including microRNA), relative to a suitable control.

Provided herein are preparations of sperm prepared by: a. enriching sperm from semen of a male subject, such as a normospermic male, sub fertile male, or oligospermic male, e.g., a subfertile (including oligospermic) male, b. incubating the sperm under energy depletion for a time suitable to potentiate the sperm, c. providing the potentiated sperm with a first energy source selected from: an effective amount of a glycolytic energy source or an effective amount of a gluconeogenesis substrate, but not an effective amount of both a glycolytic energy source and gluconeogenesis substrate.

Provided herein are sperm preservation media comprising a buffer and having a slightly acidic pH and an osmolality of between about: 300 and 400 mOsm/kg, e.g., between about: 300-380, 320-370, 330-370, 340-360, e.g., about: 320, 330, 340, 350, 360, 370, or 380, e.g., about 350, wherein the medium does not comprise a significant amount of, or in some embodiments any, egg yolk.

Provided herein are preparations of sperm prepared by: (a) incubating a mammalian sperm under energy depletion for a time suitable to generate a potentiated mammalian sperm, and (b) providing the potentiated mammalian sperm from step (a) with an effective amount of a first energy source and a second energy source in a serial manner, wherein the sperm of step (b) comprises a different epigenetic profile than a suitable control sperm.

Provided herein are methods of producing an offspring with improved fitness comprising; (a) incubating a sperm sample under energy depletion for a time suitable to generate a potentiated sperm, (b) providing the potentiated sperm with an effective amount of a first energy source, and (c) subsequently providing the sperm from step (b) with an effective amount of a second energy source, (d) fertilizing an egg with the sperm from step (c) to generate an embryo, and (e) growing the embryo in a female subject to produce the offspring with improved fitness, wherein the improved fitness comprises a reduced risk of developing a condition.

The various methods, media, preparations and kits described herein can be used combinatorially. For example, sperm preparations preserved with sperm preservation media provided by the invention can, in some embodiments, be used in the various methods provided by the invention (e.g., enhancing sperm function, promoting fertilization, producing an offspring with improved fitness, etc.), which methods can, in some embodiments, be performed using the various kits provided by the invention to then, in certain embodiments, produce the sperm preparations provided by the invention, and/or in additional methods provided by the invention, such as methods of fertilization, including methods of assisted reproduction.

Features of the methods, media, preparations of sperm, and kits described herein can include one or more of aspects of the following enumerated embodiments, which can be combined and interpolated and should not be viewed as narrow specific embodiments not amenable to combination or modulation, unless specifically provided. The examples of the instant disclosure provide non-limiting exemplification that can readily be adapted to the enumerated embodiments below:

1. A method for promoting fertilization comprising:
(a) incubating a mammalian sperm under energy depletion conditions for a time suitable to potentiate the mammalian sperm;
(b) providing the potentiated mammalian sperm from step (a) with an effective amount of a first energy source and a second energy source in a serial manner; and
(c) providing the mammalian sperm resulting from step (b) with access to an egg under conditions to promote fertilization, wherein the effective amount is an amount sufficient to induce improved sperm function.
2. The method of embodiment 1, wherein one or more sperm function selected from curvilinear velocity, amplitude of lateral head displacement, autophagy, sperm capacitation, percentage of hyperactivated sperm, percentage of intermediate motility sperm and percentage of hyperactivated sperm and intermediate motility sperm, is improved relative to a method wherein the potentiated mammalian sperm are provided with only one of the first energy source and the second energy source or with the first energy source and the second energy source simultaneously.
3. The method of embodiment 1, wherein the first energy source is a glycolytic energy source and the second energy source is a gluconeogenesis substrate, or the first energy source is the gluconeogenesis substrate and the second energy source is the glycolytic energy source, further wherein the mammalian sperm of step (a) is a human sperm.
4. The method of embodiment 3, wherein the method is performed in vitro.
5. The method of embodiment 3, wherein step (c) is performed in vivo, in the reproductive tract of a female subject by artificial insemination in the vagina or intrauterine insemination (IUI) of the mammalian sperm from step (b).
6. The method of embodiment 1, wherein providing the second energy source of step (b) is performed in vivo, in the reproductive tract of a female subject by intrauterine insemination (IUI) of the potentiated mammalian sperm provided with an effective amount of the first energy source.
7. The method of embodiment 6, wherein the first energy source is a gluconeogenesis substrate that is pyruvate and the second energy source is a glycolytic energy source.
8. The method of embodiment 4, wherein step (c) comprises incubating the mammalian sperm from step (b) with the egg, or injecting the mammalian sperm from step (b) into the cytoplasm of the egg to promote in vitro fertilization of the egg.
9. The method of embodiment 1, wherein promoting fertilization comprises generation of an embryo, wherein the embryo exhibits increased viability and/or improved implantation relative to an embryo generated by a suitable control sperm.
10. The method of embodiment 1, wherein promoting fertilization comprises generation of an embryo which develops to at least a 2-cell developmental stage, a blastocyst developmental stage, or an offspring.
11. The method of embodiment 1, wherein the mammalian sperm of step (a) is from an oligospermic subject or a subfertile subject.
12. The method of embodiment 1, wherein the mammalian sperm of step (a) is a human, non-human primate, porcine, bovine, equine, ovine, canine, feline, or murine sperm.
13. The method of embodiment 12, wherein the mammalian sperm of step (a) is a human sperm.
14. The method of embodiment 1, wherein the mammalian sperm of step (a) is a sperm recovered from a non-cryogenic or cryogenic storage.
15. The method of embodiment 1, wherein the mammalian sperm of step (a) is provided as a pool of two or more ejaculates.
16. The method of embodiment 1, wherein the mammalian sperm of step (a) is enriched from semen prior to step (a) by density gradient centrifugation, swim up, or microfluidics.
17. The method of embodiment 1, wherein the method is performed at an osmolality ranging from 200-280 mOsm/kg.
18. The method of embodiment 1, wherein step (b) further comprises providing the mammalian sperm with one or more components upstream or downstream of glycolysis in combination with at least the first energy source or the second energy source.
19. The method of embodiment 3, wherein the first energy source is selected from: (i) glucose or (ii) pyruvate; and the second energy source is selected from: (i) glucose or (ii) pyruvate, and wherein the first and second energy source are different.
20. The method of embodiment 1, wherein the incubating under energy depletion conditions of step (a) is for at least 10 minutes.
21. A method of inducing increased sperm function comprising:
(a) incubating a mammalian sperm under energy depletion conditions for a time suitable to generate a potentiated mammalian sperm;
(b) providing the potentiated mammalian sperm from step (a) with an effective amount of a first energy source selected from: (i) a glycolytic energy source or (ii) a gluconeogenesis substrate; and
(c) subsequently providing the mammalian sperm from step (b) with an effective amount of a second energy source, selected from: (i) the glycolytic energy source or (ii) the gluconeogenesis substrate, wherein the second energy source provided is not selected in step (b), wherein the effective amount is an amount sufficient to induce increased sperm function.
22. The method of embodiment 21, wherein the increased sperm function is increased relative to a suitable control sperm and wherein the suitable control sperm is an untreated mammalian sperm, the potentiated mammalian sperm of step (a) provided with an effective amount of the glycolytic energy source or the gluconeogenesis substrate independently, the potentiated mammalian sperm of step (a) provided with an effective amount of the glycolytic energy source and the gluconeogenesis substrate simultaneously, or sperm treated with standard capacitation medium (C-HTF).
23. The method of embodiment 21, which is performed in vitro.
24. The method of embodiment 21, wherein step (c) is performed in vivo, in the reproductive tract of a female subject by artificial insemination in the vagina or intrauterine insemination (IUI) of the mammalian sperm from step (b).
25. The method of embodiment 21, wherein the increased sperm function comprises an increase in motility as measured by computer assisted semen analysis (CASA).
26. The method of embodiment 25, wherein the increase in motility comprises an increase in curvilinear velocity of the mammalian sperm, increase in percentage of hyperactivated sperm, increase in percentage of intermediate motility sperm, or a combination thereof.
27. The method of embodiment 21, wherein the increased sperm function comprises an increase in sperm capacitation as measured by a sperm-zona pellucida binding assay.
28. The method of embodiment 21, wherein the increased sperm function comprises an increase in ability of the mammalian sperm to fertilize an egg as measured by a sperm penetration assay.
29. The method of embodiment 21, wherein the increased sperm function comprises generation of an embryo with increased viability, improved implantation, increased ability to develop to at least a 2-cell developmental stage, blastocyst developmental stage or an offspring relative to an embryo generated with a suitable control sperm.
30. The method of embodiment 21, wherein the mammalian sperm is a human, non-human primate, porcine, bovine, equine, ovine, canine, feline, or murine sperm.
31. The method of embodiment 30, wherein the mammalian sperm is a human sperm.
32. The method of embodiment 31, wherein the glycolytic energy source is glucose and the gluconeogenesis substrate is pyruvate.
33. A preparation of sperm comprising the mammalian sperm with increased sperm function prepared by the method of embodiment 21.
34. A preparation of sperm comprising increased percentage of hyperactivated sperm prepared by a process of:
(a) incubating a mammalian sperm under energy depletion conditions for a time suitable to generate a potentiated mammalian sperm; and
(b) providing the potentiated mammalian sperm from step (a) with an effective amount of a first energy source and a second energy source in a serial manner, wherein the effective amount is an amount sufficient to induce an increase in one or more sperm function, and wherein the preparation of sperm comprising increased percentage of hyperactivated sperm comprises an increase in one or more sperm function relative to suitable control sperm selected from:
an untreated mammalian sperm, the potentiated mammalian sperm of step (a) provided with an effective amount of the first energy source or the second energy source independently, the potentiated mammalian sperm of step (a) provided with an effective amount of the first energy source and the second energy source simultaneously, or a mammalian sperm treated with standard capacitation medium (C-HTF).
35. The preparation of sperm of embodiment 34, wherein the first energy source is a glycolytic energy source and the second energy source is a gluconeogenesis substrate, or the first energy source is the gluconeogenesis substrate and the second energy source is the glycolytic energy source.
36. The preparation of sperm of embodiment 34, wherein the mammalian sperm of step (a) is provided as a pool of two or more ejaculates.
37. The preparation of sperm of embodiment 34, wherein the mammalian sperm of step (a) is from a subfertile male or an oligospermic male.
38. The preparation of sperm of embodiment 34, wherein the mammalian sperm of step (a) is a human, non-human primate, porcine, bovine, equine, ovine, canine, feline, or murine sperm.
39. The preparation of sperm of embodiment 38, wherein the mammalian sperm of step (a) is a human sperm.
40. The preparation of sperm of embodiment 34 further comprising reduced intracellular RNA.
41. A method of inducing increased sperm function comprising;
(a) providing a mammalian sperm in a preservation medium comprising a buffer and having a pH of between about: 6-7 and an osmolality of between about: 300 and 400 mOsm/kg;
(b) incubating the mammalian sperm under energy depletion conditions for a time suitable to potentiate the mammalian sperm; and
(c) providing the potentiated mammalian sperm from step (b) with an effective amount of (i) a glycolytic energy source and/or (ii) a gluconeogenesis substrate, thereby inducing increased sperm function compared to a suitable control sperm.
42. The method of embodiment 41, wherein the glycolytic energy source and the gluconeogenesis substrate are provided simultaneously.
43. The method of embodiment 41, wherein the glycolytic energy source and the gluconeogenesis substrate are provided in a serial manner.
44. The method of embodiment 41, wherein the increased sperm function comprises increase in curvilinear velocity, amplitude of lateral head displacement, autophagy, sperm capacitation, percentage of hyperactivated sperm, percentage of intermediate motility sperm, or a combination thereof.
45. The method of embodiment 41, wherein the increased sperm function is increase in the percentage of hyperactivated sperm and intermediate motility sperm.
46. The method of embodiment 41, wherein the mammalian sperm of step (a) is provided as a pool of two or more ejaculates.
47. The method of embodiment 41, wherein the preservation medium does not comprise egg yolk.
48. The method of embodiment 41, wherein the preservation medium further comprises an antibiotic.
49. The method of embodiment 41, wherein the preservation medium further comprises a serum albumin.
50. The method of embodiment 41, wherein the buffer is HEPES, MOPS, or a combination thereof.
51. The method of embodiment 41, wherein the preservation medium has a pH of between about: 6.6-6.9 and an osmolality of between about 330-370 mOsm/kg.
52. The method of embodiment 41, wherein the preservation medium further comprises one or more carbon sources selected from the group consisting of glucose, fructose, mannose, and sucrose.
53. The method of embodiment 41, wherein the mammalian sperm in the preservation medium is stored under non-cryogenic conditions prior to step (a).
54. A method for promoting fertilization comprising:
(a) incubating a mammalian sperm under energy depletion conditions for a time suitable to potentiate the sperm, wherein prior to the incubating, the mammalian sperm is stored in a preservation medium comprising a buffer and having a slightly acidic pH and an osmolality of between about: 300 and 400 mOsm/kg;
(b) providing the potentiated sperm from step (a) with an effective amount of a first energy source and a second energy source in a serial manner;
(c) increasing one or more of sperm function, to a level greater than that obtained by providing the potentiated mammalian sperm of step (a) with an effective amount of the first energy source or the second energy source independently, or providing an effective amount of the first energy source and the second energy source simultaneously; and
(d) providing access to the mammalian sperm with increased function with an egg under conditions to promote fertilization.
55. The method of embodiment 54, wherein the mammalian sperm of step (a) is provided as a pool of two or more ejaculates.
56. The method of embodiment 54, wherein the one or more sperm function is selected from curvilinear velocity, amplitude of lateral head displacement, autophagy, sperm capacitation, percentage of hyperactivated sperm, percentage of intermediate motility sperm, and percentage of hyperactivated sperm and intermediate motility sperm.
57. The method of embodiment 54, wherein the method is performed in vitro.
58. The method of embodiment 54, wherein step (d) is performed in vivo, in the reproductive tract of a female subject by intrauterine insemination (IUI) of the mammalian sperm with increased function from step (c).
59. The method of embodiment 54, wherein providing the second energy source of step (b) is performed in vivo, in the reproductive tract of a female subject by intrauterine insemination (IUI) of the potentiated mammalian sperm provided with an effective amount of the first energy source from step (b).
60. The method of embodiment 57, wherein step (d) comprises incubating the mammalian sperm with increased function with the egg, or injecting the mammalian sperm with increased function into the cytoplasm of the egg to promote in vitro fertilization of the egg.
61. A kit comprising:
a) a first container comprising a first composition comprising a buffered sperm-potentiating energy depletion composition; and
b) a second container comprising a second composition comprising at least a first energy source suitable for a mammalian sperm,
wherein, upon incubating the mammalian sperm in the first composition for a suitable time, generates a potentiated mammalian sperm, and
wherein, upon providing the potentiated mammalian sperm an effective amount of at least the first energy source, increases function of the potentiated mammalian sperm relative to a suitable control.
62. The kit of embodiment 61, wherein the first composition comprising a buffered sperm-potentiating energy depletion composition is a nutrient-free synthetic human tubal fluid.
63. The kit of any one of embodiments 61-62, wherein the function of the potentiated mammalian sperm is curvilinear velocity, amplitude of lateral head displacement, autophagy, sperm capacitation, percentage of hyperactivated sperm, percentage of intermediate motility sperm, percentage of hyperactivated sperm and intermediate motility sperm or a combination thereof.
64. The kit of any one of embodiments 61-63, wherein the buffered sperm-potentiating energy depletion composition comprises glucose concentration of less than about: 0.5, 0.4, 0.3, 0.2, 0.1, 0.09, 0.08, 0.07, 0.06, 0.05, 0.04, 0.03 mM or less.
65. The kit of any one of embodiments 61-64, wherein the buffered sperm-potentiating energy depletion composition comprises pyruvate concentration of less than about: 0.15, 0.10, 0.09, 0.08, 0.07, 0.06, 0.05, 0.04, 0.03, 0.02, 0.01, 0.005, 0.003, 0.002 mM, or less.
66. The kit of any one of embodiments 61-65, wherein the suitable time is for at least about: 10, 20, 30, 40, 45, 50, 55, 60, 90, 120, 150, or 180 minutes.
67. The kit of any one of embodiments 61-66, wherein the buffered sperm-potentiating energy depletion composition comprises an osmolarity ranging from between about: 200-280 mOsm, e.g., between about: 220-260, 225-255, 230-250 mOsm.
68. The kit of any one of embodiments 61-67, wherein providing the potentiated mammalian sperm with an effective amount of at least the first energy source is at an osmolarity of at least about: 270, 275, 280, 285, 290, or 295 mOsm.
69. The kit of any one of embodiments 61-68, wherein the first composition comprises one or more of HEPES, e.g., about 5-20 mM, such as 7.5-12.5 mM; sodium chloride, e.g., about 80-120 mM, such as 90-100 mM; potassium chloride, e.g., about 3-8 mM, such as 4-7 mM mM, calcium chloride, e.g, about 1-5 mM, such as 1.5-2.5 mM, potassium phosphate, e.g., about 0.1-0.5 mM, such as 0.3-0.4 mM, magnesium sulfate, e.g., 0.1-0.5 mM, such as 0.16-0.35 mM, sodium bicarbonate, e.g., about 10-50 mM, such as 15-30 mM.
70. The kit of any one of embodiments 61-69, wherein the first composition further comprises phenol red, e.g., about: 0.0001%, 0.0002%, 0.0003%, 0.0004%, 0.0005%, 0.0006%, 0.0007%, 0.0008%, 0.0009%, or 0.001%.
71. The kit of any one of embodiments 61-70, wherein the first composition, and/or the second composition further comprises an antibiotic, such as gentamicin, e.g., at a concentration of about: 1-20 μg/ml, 2-18 μg/ml, 4-16 μg/ml, 6-14 μg/ml, or 8-12 μg/ml.
72. The kit of any one of embodiments 61-71, wherein the first energy source is a glycolytic energy source, or a gluconeogenesis substrate.
73. The kit of any one embodiments 61-72, wherein the second container containing the second composition comprising the at least first energy source, further comprises the buffered sperm-potentiating energy depletion composition.
74. The kit of any one of embodiments 61-73, wherein the first composition, the second composition, or both the first composition and second composition is an aqueous solution, such as a sterile aqueous solution, e.g., previously sterilized by sterile filtration.
75. The kit of any one embodiments 61-73, wherein the first composition and/or the second composition is a lyophilized composition.
76. The kit of any one of embodiments 61-75, further comprising a third container, comprising a third composition comprising at least a second energy source suitable for the mammalian sperm, wherein, upon providing the potentiated mammalian sperm an effective amount of the second energy source, increases function of the sperm, wherein the effective amount of the second energy source is provided simultaneously or sequentially with the effective amount of the first energy source.
77. The kit of embodiment 76, wherein the third composition is an aqueous solution, such as a sterile aqueous solution, e.g., previously sterilized by sterile filtration.
78. The kit of embodiment 76, wherein the third composition is a lyophilized composition.
79. The kit of any one of embodiments 76-78, wherein the third container, comprising the third composition comprising at least the second energy source suitable for the mammalian sperm, further comprises the buffered sperm-potentiating energy depletion composition.
80. The kit of any one of embodiments 76-79, wherein the third composition further comprise an antibiotic, such as gentamicin, e.g., at a concentration at a concentration of about: 1-20 μg/ml, 2-18 μg/ml, 4-16 μg/ml, 6-14 μg/ml, or 8-12 μg/ml.
81. The kit of one of embodiments 76-80, wherein the second energy source is the glycolytic energy source, or the gluconeogenesis substrate, wherein the second energy source is one that is not selected as the first energy source.
82. The kit of any one of embodiments 72-81, wherein the glycolytic energy source is glucose, e.g., at a concentration of about: 100 mM-1M, 200-900 mM, 300-800 mM, 400-600 mM or 500 mM, e.g., at least about: 100, 200, 300, 400, 500, 600, 700, 800, 900 mM, or 1M.
83. The kit of any one of embodiments 72-82, wherein the gluconeogenesis substrate is pyruvate, e.g., at a concentration of about: 10-50 mM, 15-45 mM, 20-40 mM, or 25-35 mM, e.g., at least about: 10, 15, 20, 25, 30, 35, 40, 45, or 50 mM.
84. The kit of any one of embodiments 71-83, wherein the first energy source or, optionally, the second energy source is glucose in an effective amount, wherein the effective amount of glucose is between about 0.6 mM-10.0 mM, e.g., about: 1.0-7.0 mM, 2.5-7.0 mM, 3.5-6.5 mM or about 5 mM, e.g., at least about: 1, 2, 3, or 4 mM, upon introducing into a diluent.
85. The kit of any one of embodiments 71-83, wherein the first energy source or, optionally, the second energy source is pyruvate in an effective amount, wherein the effective amount of pyruvate is between about 0.15-0.66 mM, e.g., about: 0.20-0.50 mM, 0.25-0.40 mM, or about 0.30 mM, upon introducing into a diluent.
86. The kit of any one of embodiments 71-85, wherein the first energy source is pyruvate, optionally in the form of sodium pyruvate.
87. The kit of any one of embodiments 76-86, wherein the second energy source is glucose, 88. The kit of any one of embodiments 71-87, wherein the first composition comprises human serum albumin, e.g., at a concentration of about: 1-10 mg/ml, 2-8 mg/ml, or 3-7 mg/ml.
89. The kit of any one of embodiments 71-88, wherein the kit comprises a further container comprising human serum albumin.
90. The kit of any one of embodiments 71-89, wherein the first, and/or second composition consists essentially of NaCl e.g., at a concentration of about 97.8 mM, KCl, e.g., at a concentration of about 4.7 mM, CaCl₂), e.g., at a concentration of about 2 mM, KH₂PO₄, e.g., at a concentration of about 0.37 mM, MgSO₄.7H₂O, e.g., at a concentration of about 0.2 mM, HSA, e.g., at a concentration of about 4 mg/ml, gentamycin e.g., at a concentration of about 10 μg/ml, HEPES, e.g., at a concentration of about 10 mM, and phenol red, e.g., at a concentration of about 0.0006%.
91. The kit of any one of embodiments 76-90, wherein the third composition consists essentially of NaCl e.g., at a concentration of about 97.8 mM, KCl, e.g., at a concentration of about 4.7 mM, CaCl₂), e.g., at a concentration of about 2 mM, KH₂PO₄, e.g., at a concentration of about 0.37 mM, MgSO₄.7H₂O, e.g., at a concentration of about 0.2 mM, HSA, e.g., at a concentration of about 4 mg/ml, gentamycin e.g., at a concentration of about 10 μg/ml, HEPES, e.g., at a concentration of about 10 mM, and phenol red, e.g., at a concentration of about 0.0006%.
92. The kit of any one of embodiments 71-91, further comprising a means for selecting sperm selected from: a microfluidic device, a density gradient solution, a sperm isolating matrix (such as silanized silica, optionally suspended in a nutrient-free synthetic human tubal fluid), or a combination thereof.
93. The kit of any one of embodiments 71-92, further comprising instructions for use, such as instructions for performing a method as described herein, such a starve-rescue/starve-refeed method.
94. The kit of any one of embodiments 71-93, further comprising a collection container for collecting a sample of the mammalian sperm from a mammalian donor.
95. The kit of any one of embodiments 71-94, wherein the first container and/or the second container is a bottle, a vial, a syringe, or a test tube.
96. The kit of any one of embodiments 76-95, wherein the third container is a bottle, a vial, a syringe, or a test tube.
97. The kit of any one of embodiments 71-96, wherein the first container, and/or the second container is a multi-use container.
98. The kit of any one of embodiments 76-97, wherein the third container is a multi-use container.
99. A method of increasing sperm function comprising
i) incubating the sperm under energy depletion for a time suitable to potentiate the sperm;
ii) providing the potentiated sperm with an effective amount of a first energy source selected from: a glycolytic energy source or a gluconeogenesis substrate, but not an effective amount of a glycolytic energy source and an effective amount of a gluconeogenesis substrate; and
iii) subsequently providing the sperm with an effective amount of a second energy source, so as to provide an effective amount of both a gluconeogenesis substrate and a glycolytic energy source, thereby increasing sperm function.
100. The method of embodiment 99, wherein the energy depletion comprises a low glucose concentration, e.g., less than about: 0.5, 0.4, 0.3, 0.2, 0.1, 0.09, 0.08, 0.07, 0.06, 0.05, 0.04, 0.03 mM glucose, or less, such as less than about: 0.02 or 0.01 mM, e.g., less than about 0.01 mM.
101. The method of embodiment 99 or embodiment 100, wherein the energy depletion comprises a low pyruvate concentration, e.g., less than about 0.15, 0.10, 0.09, 0.08, 0.07, 0.06, 0.05, 0.04, 0.03, 0.02, 0.01, 0.005, 0.003, 0.002 mM, or less.
102. The method of any one of embodiments 99-101, wherein the energy depletion is for at least about: 10, 20, 30, 40, 50, 60 minutes, e.g., at least about: 30, 40, 45, 50, 55, 60, 90, 120, 150, or 180 minutes, or 1, 1.5, 2, 2.5, 3, 3.5, 4, 4.5, 5, 5.5, 6, 6.5, 7, 7.5, 8, 8.5, 9, 9.5, or 10 hours.
103. The method of any one of embodiments 99-102, wherein the time between providing an effective amount of a first energy source after potentiating the sperm and providing an effective amount of a second energy source is at least about: 1, 2, 3, 4, 5, 10, 15, 20, 25, 30, 35, 40, 45, 50, 55, or 60 minutes, e.g., at least 5, 6, 7, 8, 9, 10, 11, 12, 13, 14, 15, 16, 17, 18, 19, or 20 minutes, e.g., at least between about: 5-15 minutes.
104. The method of any one of embodiments 99-103, wherein the gluconeogenesis substrate is pyruvate, e.g., at a concentration of between about: 0.15-0.66 mM, e.g., about 0.20-0.50 mM, such as about 0.25-0.40 mM, or about 0.30 mM.
105. The method of any one of embodiments 99-104, wherein the glycolytic energy source is glucose, e.g., at a concentration of between about: 0.6 mM-10.0 mM, 1.0-7.0 mM, 2.5-7.0 mM, 3.5-6.5 mM or 5 mM, e.g., at least about: 1, 2, 3, or 4 mM.
106. The method of any one of embodiments 99-105, wherein the first energy source is a glycolytic energy source, such as glucose.
107. The method of any one of embodiments 99-105, wherein the second energy source is a glycolytic energy source, such as glucose.
108. The method of any one of embodiments 99-105, wherein the first energy source is a gluconeogenesis substrate, such as pyruvate.
109. The method of any one of embodiments 99-105, wherein the second energy source is a gluconeogenesis substrate, such as pyruvate.
110. The method of any one of embodiments 99-109, wherein the sperm is a mammalian sperm (e.g., bovine, ovine, porcine, equine, feline, canine, or primate sperm, such as a human sperm.
111. The method of any one embodiments 99-110, wherein the method is performed at an osmolarity ranging from between about: 200-280 mOsm, e.g., between about: 220-260, 225-255, 230-250 mOsm during energy depletion, optionally, wherein upon addition of an effective amount of the first and/or second energy source, the osmolarity is increased to at least about: 270, 275, 280, 285, 290, or 295 mOsm.
112. The method of any one of embodiments 99-111, further comprising one or more quantitative assessments of sperm motility or quality, e.g., by CASA or measuring DNA fragmentation (e.g., by TUNEL), lipid peroxidation, reactive oxygen species, or a combination thereof.
113. The method of any one of the preceding embodiments, wherein prior to treatment, the sperm are recovered from cryogenic storage.
114. The method of any one of embodiments 99-112, wherein prior to treatment, the sperm are recovered from non-cryogenic storage.
115. The method of any one of embodiments 99-114, wherein the sperm are pooled from two or more ejaculates (e.g., 2, 3, 4, 5, 6, or more ejaculates).
116. The method of any one of embodiments 99-115, wherein the sperm is obtained from a subfertile male or an oligospermic male, e.g., with a sperm count of less than about 15 million sperm per milliliter.
117. The method of any one of embodiments 99-116, wherein the sperm are enriched (or isolated) from semen prior to energy depletion, e.g., by density gradient centrifugation, swim up, or microfluidics.
118. The method of any one of embodiments 99-117, further comprising providing the sperm to a female reproductive tract, optionally wherein the effective amount of the second energy source is provided in the female reproductive tract.
119. The method of any one of embodiments 99-118, wherein the method is performed in vitro.
120. The method of embodiment 119, further comprising contacting the sperm with increased function with an egg under conditions to promote fertilization.
121. A method of fertilization comprising providing sperm prepared by the method of any one of embodiments 99-117, with access to an egg (including by, for example, ICSI) for a time sufficient to fertilize the egg.
122. The method of any one of embodiments 99-121, wherein relative to a suitable control, there is an increase in hyperactivated and/or intermediate motility sperm of at least about: 30, 35, 40, 45, 50, 55, 60, 65, 70, 75, 80, 85, 90, 95, or 100%, or more, such as about 1.1, 1.2, 1.3, 1.4, 1.5, 1.6, 1.7, 1.8, 1.9, 2, 3, 4, 5-fold, or more, including about 1.2-fold.
123. The method of any one of embodiments 99-122, further comprising a step of providing the sperm with one or more components upstream or downstream of glycolysis such as NADH, NAD+, citrate, AMP, or ADP, in combination with at least the first energy source or the second energy source.
124. A preparation of hyperactivated sperm comprising at least 5% hyperactivated sperm, optionally wherein the preparation has not been previously sorted on the basis of hyperactivation, optionally wherein the hyperactivated and/or intermediate sperm have 10, 15, 20, 25, 30, 35, 40, 45, 50%, or more (e.g., 2, 3, 4, 5, 6, 7, 8, 9, 10-fold, or more) reduction in intracellular RNA. concentration (such as small non-coding RNA, including microRNA), relative to a suitable control.
125. A preparation of sperm prepared by:
a. enriching sperm from semen of a male subject, such as a normospermic male, sub fertile male, or oligospermic male, e.g., a subfertile (including oligospermic) male;
b. incubating the sperm under energy depletion for a time suitable to potentiate the sperm;
c. providing the potentiated sperm with a first energy source selected from: an effective amount of a glycolytic energy source or an effective amount of a gluconeogenesis substrate, but not an effective amount of both a glycolytic energy source and gluconeogenesis substrate.
126. A method of fertilization comprising providing the preparation of embodiment 125 with access to an egg and an effective amount of a second energy source so as to provide an effective amount of both a gluconeogenesis substrate and a glycolytic energy source for a time sufficient to fertilize the egg.
127. The method of embodiment 126, which is performed in vitro.
128. The method of embodiment 126, which is performed in vivo, in the reproductive tract (vagina or uterus) of a female.
129. The preparation of embodiment 125, wherein the sperm are from an oligospermic subject or subfertile (e.g., low sperm motility) subject.
130. The preparation of embodiment 125, prepared by the method of any one of embodiments 99-120.
131. An article of manufacture comprising:
i) a sperm potentiating solution that, upon contact with sperm, induces energy depletion;
ii) a solution providing a first energy source selected from: an effective amount of a glycolytic energy source or an effective amount of a gluconeogenesis substrate, but not an effective amount of both a glycolytic energy source and a gluconeogenesis substrate; and
iii) a solution providing an effective amount of a second energy source.
132. The article of manufacture of embodiment 131, further comprising a sperm isolating matrix.
133. The article of manufacture of embodiment 132, wherein the sperm isolating matrix is silanized silica, optionally wherein the silanized silica is in media substantially free of any glycolytic energy source or gluconeogenesis substrate.
134. A sperm preservation medium comprising a buffer and having a slightly acidic pH and an osmolality of between about: 300 and 400 mOsm/kg, e.g., between about: 300-380, 320-370, 330-370, 340-360, e.g., about: 320, 330, 340, 350, 360, 370, or 380, e.g., about 350, wherein the medium does not comprise a significant amount of, or in some embodiments any, egg yolk.
135. The sperm preservation medium of embodiment 134, further comprising a carbon source, optionally wherein the carbon source is selected from glucose, fructose, mannose, sucrose, or a combination thereof.
136. The sperm preservation medium of embodiment 135, wherein the carbon source is glucose.
137. The sperm preservation medium of embodiment 136, wherein the glucose is present at a concentration of between about: 0.1-0.4 M, such as between about 0.2-0.4 M, e.g., between about: 0.30-0.36 M or about 0.33 M.
138. The sperm preservation medium of any one of embodiments 134-137, wherein the buffer is a zwitterionic buffer, wherein the buffer concentration is between about: 1 and 100 mM, e.g., 1 and 50 mM, 1 and 40 mM, 1 and 30 mM, 1 and 20 mM, 5-15 mM; e.g., about: 2, 3, 4, 5, 6, 7, 8, 9, 10, 11, 12, 13, 14, 15, 16, 17, 18, 19, or 20 mM, e.g., about 10 mM.
139. The sperm preservation medium of embodiment 138, wherein the buffer is 4-(2-hydroxyethyl)-1-piperazineethanesulfonic acid (HEPES), 3-(N-morpholino) propanesulfonic acid (MOPS), or a combination thereof.
140. The sperm preservation medium of any one of embodiments 134-139, further comprising an antibiotic.
141. The sperm preservation medium of embodiment 140, wherein the antibiotic is an aminoglycoside.
142. The sperm preservation medium of embodiment 140 or 141, wherein the antibiotic is gentamicin.
143. The sperm preservation medium of embodiment 142, wherein the gentamicin is present at a concentration of between about: 5 and 20 μg/ml, e.g., about 10 μg/ml.
144. The sperm preservation medium of any one of embodiments 131-143, further comprising a serum albumin.
145. The sperm preservation medium of embodiment 144, wherein the serum albumin is bovine serum albumin (BSA) or human serum albumin (HSA), or a combination thereof, more particularly wherein the serum albumin is present at a concentration of about: 1.5-4.5% (W/V), e.g., about: 2-4%, 2.5-3.5%, or 3%.
146. The sperm preservation medium of any one of embodiments 134-145, having an osmolality of between about 340-360 mOsm/kg.
147. The sperm preservation medium of any one of embodiments 134-146, having a pH of between about: 6-7, e.g., 6.6-6.9.
148. The sperm preservation medium of any one of embodiments 134-147, further comprising one or more of a sterol, an antioxidant, or an anti-inflammatory agent.
149. A sperm preservation medium comprising a zwitterionic buffer and pH of between about: 6.6 and 6.9, glucose at a concentration of between about: 0.25-0.36 M, and osmolality of between about: 320-380 mOsm/kg, wherein the medium does not comprise a significant amount of, or in some embodiments any, egg yolk.
150. The sperm preservation medium of embodiment 149, further comprising an antibiotic, optionally wherein the antibiotic is gentamicin.
151. The sperm preservation medium of embodiment 149 or embodiment 150, wherein the pH is about 6.8, the glucose concentration is about 0.330 mM, the osmolality is about 350 mOsm/kg, and wherein the serum albumin is BSA and/or HSA, optionally wherein the BSA and/or HSA is present at a concentration of about: 2-4% (W/V).
152. The sperm preservation medium of embodiment 151, wherein the buffer is HEPES, MOPS, or a combination thereof.
153. The sperm preservation medium of any one of embodiments 149-152 provided as a sterile formulation, optionally in a sealed sterile container.
154. The sperm preservation of embodiment 153, wherein the medium is lyophilized.
155. The sperm preservation medium of embodiment 153, wherein the medium is a liquid formulation.
156. The sperm preservation medium of any one of embodiments 149-155, wherein sperm stored in the preservation medium for up to 4, 5, 6, 7, 8, 9, 10, 11, 12 days, or more, at about 4° C., maintain at least about: 40, 45, 50, 55, 60, 65, 70, 75, 80%, or more, motile sperm upon transfer to capacitation medium, relative to suitable control sperm.
157. The sperm preservation medium of any one of embodiments 149-156, wherein sperm stored in the preservation medium for 7 days at about 4° C., have at least about: 40, 45, 50, 55, 60, 65, 70, 75, 80%, or more, motile sperm upon transfer to capacitation medium, relative to suitable control sperm.
158. The sperm preservation medium of any one of embodiments 149-157, wherein sperm stored in the preservation medium for 4, 5, 6, 7, 8, 9, 10, 11, 12 days, or more, at about 4° C., have at least about 75% motile sperm upon transfer to capacitation medium, relative to suitable control sperm.
159. The sperm preservation medium of any one of embodiments 149-158, wherein sperm stored in the preservation medium for 7 days at about 4° C. have, upon transfer to capacitation medium, a percent motile sperm that is no more than 1, 2, 5, 10, 15, or 20% reduced, relative to control sperm before storage in the preservation medium.
160. The sperm preservation medium of any one of embodiments 156-159, wherein sperm stored in the medium exhibit one or more of:
reduced TUNEL staining of at least 30, 40, 50, 55, 60, 65, 70, 80, 85, 90, 95%, or more, relative to cryogenically stored cells;
reduced lipid peroxidation of at least 30, 40, 50, 55, 60, 65, 70, 80, 85, 90, 95%, or more, relative to cryogenically stored cells;
reduced reactive oxygen species of at least 30, 40, 50, 55, 60, 65, 70, 80, 85, 90, 95%, or more, relative to cryogenically stored cells; or
a combination of the foregoing, including 1, 2, or all 3.
161. A sterile liquid sperm preservation medium having a pH of between about: 6.7 and 6.9, consisting essentially of a zwitterionic buffer, glucose at a concentration of between about: 0.25-0.35 M, an antibiotic, osmolality of between about 340-360 mOsm/kg, BSA or HSA at a concentration of about: 2-4% (W/V), wherein sperm stored in the preservation medium for up to 12 days at 4° C. maintain at least 60% motile sperm upon transfer to capacitation medium, relative to suitable control sperm; optionally wherein the medium does not contain egg yolk.
162. A composition comprising the sperm preservation medium of any one of the preceding embodiments, further comprising live sperm, optionally wherein the sperm are enriched from semen (e.g., by density gradient centrifugation, swim up, filtration, or microfluidics).
163. The composition of embodiment 162, wherein the sperm is mammalian sperm, such as bovine, ovine, equine, porcine, leporine, feline, canine, or primate sperm, such as human.
164. The composition of embodiment 163, wherein the mammalian sperm is from a subject with reduced sperm count, e.g., less than about 15 million sperm per milliliter.
165. The composition of any one of embodiments 162-164, wherein when stored for up to 4, 5, 6, 7, 8, 9, 10, 11, 12 days, or more, at about 4° C., at least about: 40, 45, 50, 55, 60, 65, 70, 75, 80%, or more, of the sperm are motile upon transfer to capacitation medium, relative to suitable control sperm.
166. A composition comprising:
(i) sperm, e.g., human sperm, and
(ii) a buffer,
wherein the composition has a pH of between 5 and 7 (e.g., 6-7 or 6.6-6.9), and an osmolality of between about: 300 and 400 mOsm/kg (e.g., between about: 300-380, 320-370, 330-370, 340-360, e.g., about: 320, 330, 340, 350, 360, 370, or 380, e.g., about 350) wherein the medium optionally does not comprise a significant amount of, or in some embodiments any, egg yolk.
167. The composition of embodiment 166, wherein the non-sperm portion of the composition is the sperm preservation medium of any one of embodiments 134-161.
168. A composition comprising human sperm and liquid sperm preservation medium, the liquid sperm preservation medium having a pH of between about: 6.7 and 6.9, consisting essentially of a zwitterionic buffer, glucose at a concentration of between about: 0.25-0.35 M, osmolality of between about 340-360 mOsm/kg, an antibiotic, BSA or HSA at a concentration of about: 2-4% (W/V), wherein when stored for up to 12 days at 4° C., at least 60% of sperm are motile upon transfer to capacitation medium, relative to suitable control sperm; optionally wherein the medium does not contain egg yolk.
169. The composition of any one of embodiments 134-168, wherein the sperm are pooled from two or more ejaculates, (e.g., 2, 3, 4, 5, 6, or more ejaculates).
170. A method of preserving sperm comprising contacting sperm with the medium of any one of embodiments 134-161.
171. A method of fertilization comprising introducing to the reproductive system (e.g., vagina or uterus) of a female recipient, the composition of any one of embodiments 162-169, optionally wherein the sperm are isolated from the composition and placed in a capacitation medium before introduction to the reproductive system of the female recipient.
172. A method of fertilization comprising contacting an egg with the composition of any one of embodiments 162-169 (including, for example, by injection, such as by ISCI), optionally wherein the sperm are isolated from the composition and placed in a capacitation medium before contacting the egg.
173. A preparation of sperm prepared by:
(a) incubating a mammalian sperm under energy depletion for a time suitable to generate a potentiated mammalian sperm; and
(b) providing the potentiated mammalian sperm from step (a) with an effective amount of a first energy source and a second energy source in a serial manner, wherein the sperm of step (b) comprises a different epigenetic profile than a suitable control sperm.
174. The preparation of sperm of embodiment 173, wherein the suitable control sperm is an untreated mammalian sperm, the potentiated mammalian sperm of step (a) provided with an effective amount of the first energy source or the second energy source independently, the potentiated mammalian sperm of step (a) provided with an effective amount of the first energy source and the second energy source simultaneously, or sperm treated with standard capacitation medium (C-HTF).
175. The preparation of sperm of any one of embodiments 173-174, wherein the different epigenetic profile comprises an altered level of DNA methylation, DNA acetylation, RNA methylation, protein (e.g, histone) methylation, protein (e.g., histone) acetylation, or a combination thereof.
176. The preparation of sperm of any one of embodiments 173-175, wherein the sperm of step (b) further comprises a reduced RNA level relative to the suitable control sperm.
177. The preparation of sperm of embodiment 176, wherein the reduced RNA level comprises a reduction in non-coding RNA (ncRNA).
178. The preparation of sperm of embodiment 177, wherein the non-coding RNA is miRNA.
179. A method of producing an offspring with improved fitness comprising;
(a) incubating a sperm sample under energy depletion for a time suitable to generate a potentiated sperm;
(b) providing the potentiated sperm with an effective amount of a first energy source; and
(c) subsequently providing the sperm from step (b) with an effective amount of a second energy source;
(d) fertilizing an egg with the sperm from step (c) to generate an embryo; and
(e) growing the embryo in a female subject to produce the offspring with improved fitness, wherein the improved fitness comprises a reduced risk of developing a condition.
180. The method of embodiment 179, wherein the offspring with improved fitness does not develop the condition.
181. The method of any one of embodiments 179-180, wherein the sperm of step (c) comprises a different epigenetic profile than a suitable control sperm.
182. The method of any one of embodiments 179-181, wherein the sperm of step (c) comprises reduced intracellular RNA levels than a suitable control sperm.
183. The method of any one of embodiments 179-182, wherein the condition is obesity or an obesity-associated disorder (e.g., type 2 diabetes, cardiovascular disease, respiratory disease, infertility or cancer).
184. The method of any one of embodiments 179-183, wherein the first energy source is a glycolytic energy source and the second energy source is a gluconeogenesis substrate.
185. The method of any one of embodiments 179-183, wherein the first energy source is a gluconeogenesis substrate and the second energy source is a glycolytic energy source.

### BRIEF DESCRIPTION OF THE DRAWINGS

FIG. 1 is a bar graph of the percentage of hyperactive and intermediate motility sperm in control and starved (glucose, pyruvate, and lactate-free) conditions.

FIG. 2 is a bar graph of the percentage of hyperactive and intermediate motility sperm in control and starved (glucose, pyruvate, and lactate-free) conditions following: addition of glucose and pyruvate together (Starve/Rescue simultaneous), glucose only (Starve/Glucose rescue), pyruvate only (Starve/Pyruvate only), 1 hour glucose+15 minutes pyruvate (Starve/glucose rescue+15 minute pyruvate), or 1 hour pyruvate+15 minute Glucose (Starve/pyruvate rescue+15 minute Glucose).

FIG. 3 is an illustration of density gradient isolation of sperm coupled to certain exemplary embodiments of methods provided by the invention.

FIG. 4 is an illustration of swim-up isolation of sperm coupled to certain exemplary embodiments of methods provided by the invention.

FIG. 5A is a bar graph of the percentage of intermediate motility sperm+/−SEM in 7 different donors N=20, *: p<0.05 relative to control as determined by t-test. Semen samples were obtained from healthy volunteers.

FIG. 5B is a bar graph of the percentage of hyperactive motility sperm+/−SEM in 7 different donors N=20, *: p<0.05 relative to control as determined by t-test. Semen samples were obtained from healthy volunteers.

FIG. 6A is a bar graph of the percentage of intermediate motility sperm+/−SEM. N=5, *: p<0.05 relative to control as determined by t-test. Semen samples were obtained from men seeking treatment for infertility.

FIG. 6B is a bar graph of the percentage of hyperactive motility sperm+/−SEM. N=5, *: p<0.05 relative to control as determined by t-test. Semen samples were obtained from men seeking treatment for infertility.

FIG. 7 illustrates general practices for IVF and IUI. Sperm processing for IVF (left) generally consists of a separation step, such as density gradient centrifugation (pictured here), followed by a washing step. Swim-up (not pictured) is sometimes used as an alternative means of separation. Sperm processing for IUI (right) requires only a washing step, but some clinics prefer to include an initial separation step via either density gradient centrifugation (pictured) or swim-up (not pictured).

FIG. 8 provides an overview of sperm processing for IVF using an exemplary kit of the invention. In one exemplary application for IVF, a kit for nutrient-free density gradient centrifugation can be utilized with a kit for nutrient free wash, incubation, and sequential nutrient addition. This allows a sperm preparation to be prepared with nutrient free separation, washing and incubation, followed by staged re-addition of glucose and pyruvate. After the final glucose incubation, the sample is centrifuged and resuspended in the appropriate volume of fertilization medium, which can, for example, be the clinic's preferred fertilization medium prior to oocyte co-incubation.

FIG. 9 provides an overview of sperm processing for IUI using an exemplary kit of the invention. In one exemplary application for IUI, a kit for nutrient-free density gradient centrifugation can be utilized with a kit for for nutrient free wash, incubation, and single nutrient addition. This allows a sperm preparation to be prepared with nutrient free separation, washing and incubation, followed by staged re-addition of pyruvate. For IUI, glucose is not reintroduced prior to insemination, since it is present in the uterus.

FIG. 10 shows effects of starve-rescue protocol. Common sperm preparations were compared to a starve-rescue protocol wherein human sperm were initially incubated in the absence of glucose and pyruvate. Shortly after the glucose and pyruvate were reintroduced (C), a greater percentage of sperm exhibited intermediate and hyperactivated motility phenotypes (representative traces of each are shown in the inset images). N=20 (samples from 7 individuals). * p=0.0084.

FIG. 11 shows the number of 2-cell and blastocyst-stage embryos obtained. N=4 (C57BL/6J mice: subfertile mouse strain), * P<0.005, ** P=0.17. Transfer of these embryos to females also yielded a more than 3-fold increase in live birth rate.

FIG. 12 provides an overview of sperm processing using a nutrient-free sHTF (here depicted as “sHTF medium” or “sHTF wash buffer”), a component of a kit for IVF and a kit for IUI, for swim-up protocol sperm separation. Aligning with common practice, the semen is carefully layered beneath sHTF medium with a pipette and incubated to allow motile sperm to swim upward, out of the semen, and into the overlying medium. After the incubation, the upper sHTF medium containing motile sperm is carefully transferred to the wash step of a kit for IVF or a kit for IUI to complete sperm preparation.

FIG. 13A is a line graph of the percentage of motile sperm recovered after storage at 4° C. Sperm stored in either Test preservation Medium or in EFM for the time indicated on the x-axis were recovered and motility following capacitation was measured. Data is shown as a percentage of the total motile sperm at time zero (acquired shortly after sample processing).

FIG. 13B is a line graph of the percentage motile sperm recovered after storage at 4° C. Sperm stored in either Test preservation Medium or in Refrigeration medium at 4° C. for the time indicated on the x-axis were recovered and motility following capacitation was measured. Data is shown as a percentage of the total motile sperm at time zero (acquired shortly after sample processing).

FIG. 14A is bar graph of the percentage of sperm with DNA fragmentation as determined by TUNEL staining following storage for 7 days in Test Medium (preservation medium) at 4° C. or cryopreservation.

FIG. 14B is a scatter plot of the percentage motile sperm recovered after 7 days of storage in Test Medium (preservation medium) at 4° C. compared to cryopreservation (CRYO). Sperm were recovered, and motility was assessed following capacitation. Data are shown as a percentage of the total motile sperm at time zero, with each data point representing an individual measurement.

### DETAILED DESCRIPTION OF THE INVENTION

Male factor is a contributing factor for ˜50% of couples having difficulty conceiving. Low sperm count is a recognized factor in male infertility. The World Health Organization defines low sperm count (oligospermia) as less than 15 million sperm per milliliter (Cooper et al., Human Reproduction Update, 16(3), 231-245, 2009). Other factors contributing to male infertility or subfertility include low motility or abnormal morphology. An important aspect of assisted reproduction is obtaining maximal function of male gametes (sperm) to help maximize fertilization. Before fertilization, sperm must go through a series of changes to be able to fertilize the egg, a process called sperm capacitation. In vitro capacitation media, includes three components (albumin, calcium and bicarbonate) and initiate sperm capacitation. Sperm initially swim progressively with an almost symmetrical flagellar movement. After different periods of time, which depend on the species, the straight sperm movement is replaced by an in-place helical movement known as “hyperactivation”. While methods for activating sperm exist, they fail to achieve maximal sperm activation and therefore do not adequately address the impact of male factor in infertility. Accordingly, a need exists for media, compositions, and methods for increasing sperm function, e.g., to facilitate assisted reproduction.

The present disclosure provides, inter alia, methods for preserving sperm, methods for increasing sperm function, methods for promoting fertilization, kits for performing such methods, preparations of sperm, and articles of manufacture. The invention is based, at least in part, on Applicant's surprising discovery that staged reintroduction of different energy sources after a period of starvation achieves superior activation of sperm.

### Definitions

To facilitate an understanding of the present disclosure, a number of terms and phrases are defined below.

The terms “increased”, “increase”, “increasing” or “enhance” or “promote” are all used herein to generally mean an increase; for the avoidance of doubt, the terms “increased”, “increase”, or “enhance”, mean an increase of at least 5%, e.g., at least 10% as compared to a suitable control, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a suitable control, or at least about a 2-fold, or at least about a 3-fold, or at least about a 4-fold, or at least about a 5-fold or at least about a 10-fold increase, or any increase between 2-fold and 10-fold or greater as compared to a suitable control. The increase can be, for example, at least 10%, at least 20%, at least 30%, at least 40% or more, and is preferably to a level accepted as within the range of normal sperm from a mammalian male subject without a given disease (e.g., male infertility, due to abnormal sperm function or oligospermia).

The terms, “decrease”, “reduce”, “reduction”, “lower” or “lowering,” or “inhibit” are all used herein generally to mean a decrease. For example, “decrease”, “reduce”, “reduction”, or “inhibit” means a decrease by at least 5%, e.g., 10% as compared to a suitable control, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a suitable control), or any decrease between 10-100% as compared to a suitable control. The decrease can be, for example, at least 10%, at least 20%, at least 30%, at least 40% or more, than the range of normal for an individual without a given disease.

As used herein, the term “effective amount” means the total amount of the active component(s) of a first energy source or a second energy source that is sufficient to cause a change on a detectable function of the mammalian sperm (e.g., sperm motility, curvilinear velocity, amplitude of lateral head displacement, autophagy, sperm capacitation, percentage of hyperactivated sperm, percentage of intermediate motility sperm and percentage of hyperactivated sperm and intermediate motility sperm, ability to fertilize an egg, and generation of an embryo). When applied to an individual energy source, administered alone, the term refers to that energy source alone. When applied to a combination, the term refers to combined amounts of the first energy source and the second energy source that result in the effect, whether administered in combination, serially or simultaneously.

The term “an effective amount” includes within its meaning a sufficient amount of an energy source (e.g., a gluconeogenesis substrate or glycolytic energy source) to provide the desired effect. As it relates to the present disclosure, the desired effect can be increase in one or more sperm function or increase in fertilization. The exact amount required will vary depending on factors such as the mammalian sperm species being treated, the age and general condition of the male subject from whom the mammalian sperm is obtained, for example if the sperm is obtained from a sub-fertile mammalian subject. Thus, it is not possible to specify an exact “effective amount”. However, for any given case, an appropriate “effective amount” may be determined by one of ordinary skill in the art using only routine experimentation.

The term “spermatozoon” refers to a live reproductive cell from a male mammal. The term “spermatozoa” refers to a plurality of live male reproductive cells. Unless required otherwise by context, the plural and singular forms are interchangeable. The term “sperm” is used as an abbreviation and refers to at least one spermatozoon.

As used herein, the term “ability to fertilize an egg” refers to ability of a sperm (e.g., mammalian sperm) to penetrate an unfertilized egg (ovum) resulting in combination of their genetic material resulting in the formation of a zygote. As it relates to the present disclosure, the “ability to fertilize” an egg can be ability to fertilize in vitro and/or in vivo. In some embodiments, ability to fertilize in vitro comprises fertilization by natural conception, intravaginal insemination, intrauterine insemination, or intracytoplasmic sperm injection (ICSI).

The term “embryo” is used herein to refer both to the zygote that is formed upon fertilization of an unfertilized egg by a mammalian sperm, to form a diploid totipotent cell, e.g. a fertilized egg and to the embryo that undergoes subsequent cell divisions to develop to 2-cell stage or greater (e.g., 4-cell stage, 16-cell stage, 32-cell stage, the blastocyst stage (with differentiated trophectoderm and inner cell mass) or development into an offspring).

As used herein, the term “ability to develop” refers to the ability or capacity of an embryo to grow or develop. The terms may refer to the ability or capacity of an embryo to reach at least the 2-cell developmental stage, the blastocyst developmental stage, implant into the uterus, to develop to a full offspring, or be born live. The term “offspring” as used herein refers to a progeny of a parent, wherein the progeny is an unborn fetus or a newborn.

The term “blastocyst” refers to an embryo, five or six days after fertilization, having an inner cell mass, an outer cell layer called the trophectoderm, and a fluid-filled blastocele cavity containing the inner cell mass from which the whole of the embryo is derived. The trophectoderm is the precursor to the placenta. The blastocyst is surrounded by the zona pellucida which is subsequently shed when the blastocyst “hatches.” The zona pellucida, composed of a glycoprotein coat, surrounds the oocyte from the one-cell stage to the blastocyst stage of development. Prior to embryo attachment and implantation, the zona pellucida is shed from the embryo by a number of mechanisms including proteolytic degradation. The zona pellucida functions initially to prevent entry into the oocyte by more than one sperm, then later to prevent premature adhesion of the embryo before its arrival into the uterus.

As used herein, the term “enriched” refers to a composition or fraction or preparation wherein an object species has been partially purified such that the concentration of the object species is substantially higher than the naturally occurring level of the species in a finished product or preparation without enrichment.

The term “assisted reproductive technologies” or “ART” or “assisted fertilization” has its general meaning in the art and refers to methods used to achieve pregnancy by artificial or partially artificial means. Assisted reproductive technologies include but are not limited to classical in vitro fertilization (IVF), intracytoplasmic sperm injection (ICSI), intrauterine insemination (IUI), and intracervical insemination.

The term “intrauterine insemination” or “IUI” refers to intrauterine injection of sperm or spermatozoa directly into a uterus.

The term “in vitro fertilization” or “IVF” refers to a process by which oocytes are fertilized by sperm outside of the body, in vitro. IVF is a major treatment in infertility when in vivo conception has failed.

The term “intracytoplasmic sperm injection” or “ICSI” refers to an in vitro fertilization procedure in which a single sperm is injected directly into the cytoplasm of an egg. This procedure is most commonly used to overcome male infertility factors, although it may also be used where oocytes cannot easily be penetrated by sperm, and occasionally as a method of in vitro fertilization.

Some numerical values disclosed throughout are referred to as, for example, “X is at least or at least about 100; or 200 [or any numerical number].” This numerical value includes the number itself and all of the following:
i. X is at least 100;
ii. X is at least 200;
iii. X is at least about 100; and
iv. X is at least about 200.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i. X being administered on between day 1 and day 2;
ii. X being administered on between day 2 and day 3;
iii. X being administered on between about day 1 and day 2;
iv. X being administered on between about day 2 and day 3;
v. X being administered on between day 1 and about day 2;
vi. X being administered on between day 2 and about day 3;
vii. X being administered on between about day 1 and about day 2; and
viii. X being administered on between about day 2 and about day 3.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

### Sperm Function

In some embodiments, provided herein is a method for increasing sperm function. The method comprises incubating a mammalian sperm under energy depletion for a time suitable to potentiate the mammalian sperm, providing the potentiated mammalian sperm with an effective amount of a first energy source selected from: (i) a glycolytic energy source or (ii) a gluconeogenesis substrate, and subsequently providing the mammalian sperm from step (b) with an effective amount of a second energy source, selected from: (i) the glycolytic energy source or (ii) the gluconeogenesis substrate, wherein the energy source provided is not the one selected as first energy source, thereby inducing increased sperm function compared to a suitable control sperm. In some embodiments, the method is performed in vitro. In some embodiments, the providing of the second energy source is performed in vivo, for example, by cervical or intrauterine insemination of the sperm which has been previously incubated under energy depletion and provided a first energy source. Increased sperm function includes one or more of: increased motility such as the percentage of sperm in a population exhibiting hyperactivation and/or intermediate motility as assessed by CASAnova (see Goodson et al., 2017, Biol. Reprod. 97:698-708; doi:10.1093/biolre/iox120), increased autophagy, increased capacitation, and increased rates of fertilization, e.g., development to at least two cells, blastocyst development, or live birth. Accordingly, in some embodiments, sperm function can be sperm motility, curvilinear velocity, amplitude of lateral head displacement, autophagy, sperm capacitation, percentage of hyperactivated sperm, percentage of intermediate motility sperm and percentage of hyperactivated sperm and intermediate motility sperm, ability to fertilize an egg, generation of an embryo. In some embodiments, the embryo generated by the sperm with increased function comprises one or more characteristics selected from increased viability, increased implantation, increased ability to develop to a at least a 2-cell developmental stage, blastocyst developmental stage, an offspring—i.e., a live birth, or an offspring with improved fitness (e.g., improved fitness comprising a reduced risk of developing a condition).

In some embodiments, the first and second energy sources are provided in a serial manner (e.g., providing a first energy source and subsequently providing a second energy source). In some embodiments, the first and second energy sources are provided simultaneously. An increase in one or more sperm functions, as contemplated herein, constitutes an increase in the one or more sperm functions relative to a suitable control sperm. In some embodiments, the one or more sperm functions can be increased by at least or at least about: 5%, 10%, 20%, 30%, 40%, 50%, 60%, 75%, 80%, 90%, or 100%, 200%, 300% or more. In some embodiments, the one or more sperm functions can be increased by from 10% to 200%, from 25% to 150%, from 50% to 100%, or from 70% to 90%.

Provided herein, are methods to increase sperm function and preparation of sperm comprising increased function relative to a suitable control sperm. As it relates to the present disclosure, sperm “activity” and/or “function” encompass physiological processes such as, for example, sperm motility, sperm tropism (namely, the tendency of sperm to move towards or away from certain stimuli), and ability to fertilize an egg. The terms “activity” and/or “function” can further include processes which occur prior to, during fertilization and/or interaction with the egg (or membranes/layers thereof)—such processes may include, for example sperm capacitation and acrosomal activity, and/or processes after fertilization of egg, for example, formation of an embryo. In some embodiments, the embryo exhibits increased (longer) viability, improved implantation, and/or ability to develop to a 2-cell stage, a blastocyst, or to an offspring resulting in live birth.

Exemplary methods to measure an increase in sperm function may be assessed by motility, mucus penetration, oocyte fertilization or subsequent embryonic development and the like. Methods to determine sperm function are well known in the art, see for example, SS. Vasan Indian J Urol. 2011 January-March; 27(1): 41-48.

### Sperm Motility

With regard to sperm motility, one of skill will appreciate that the term “motility” not only relates to general movement, but may be applied to other aspects of motility such as, for example, the speed of movement of a sperm cell and/or any increase or decrease in the proportion of moving sperm cells in any given population. As such, the methods disclosed herein may be used not only to increase sperm motility, but also to increase the speed of movement of a sperm cell and/or the proportion (percentage) of moving cells in any given population of sperm.

Motility of sperm is expressed as the total percent of motile sperm, or the velocity of sperm that are motile. These measurements may be made by a variety of assays, but are conveniently assayed in one of two ways. Either a subjective visual determination is made using a phase contrast microscope when the sperm are placed in a hemocytometer or on a microscope slide, or a computer assisted semen analyzer is used. Under phase contrast microscopy, motile and total sperm counts are made and speed is assessed as fast, medium or slow. A second method of assessing sperm motility is by using a computer assisted semen analyzer (Hamilton Thorn, Beverly, Mass.), the motility characteristics of individual sperm cells in a sample are objectively determined. Briefly, a sperm sample is placed onto a slide or chamber designed for the analyzer. The analyzer tracks individual sperm cells and determines motility and velocity of the sperm. Data is expressed as percent motile, and measurements are obtained for path velocity and track speed as well.

Accordingly, the term “motility” encompasses percentage of motile sperm which can be the percentage of the total number of sperm assessed that fall within all World Health Organization (WHO) categories of motility except the category designated “no motility” regardless of velocity or directionality as discussed in Cooper et al. Human Reproduction update, Vol 16, No 3 pp 231-245, 2010. Manual counting classifies sperm cells into 4 categories (immotile, locally motile, non linear and linear motile) using qualitative subjective criteria of selection.

The term “motility” encompasses percentage of motile sperm i.e., the percentage of total number of sperm assessed in a population exhibiting progressive motility, hyperactivated motility and/or intermediate motility based on Computer assisted sperm analysis (For example, as assessed by CASAnova; see Goodson et al, 2017, Biol. Reprod. 97:698-708).

The methods disclosed herein can increase percentage of progressive motility sperm, i.e., percentage of sperm exhibiting linear movement from one point to another, with turns of the head of less than 90 degrees from sperm that are otherwise non-progressive, i.e., sperm that move but do not make forward progression. In some embodiments, the methods disclosed herein can increase percentage of intermediate motility sperm. Intermediate motility sperm is characterized by movement that is similar to progressive vigorous motility, but has a larger variance from the path and turns of the sperm head of approximately 90 degrees, such as an oscillating movement. In some embodiments, the increased motility comprises increase in percentage of activated hyperactive sperm, also known as hyperactivated sperm. Hyperactivated sperm motility is characterized by sperm that have a high amplitude, asymmetrical beating pattern of the flagellum. Hyperactivated motility is characterized by vigorous movement with many seemingly random variations without a well-defined progressive path and turns of the sperm head of greater than 90 degrees. Hyperactivated sperm motility is more vigorous and short term than progressive motility. Biologically, hyperactivated sperm motility is important to enable sperm to traverse the egg outer investments prior to fertilizing the mature egg. In some embodiments, the methods disclosed herein can increase percentage of hyperactivated sperm and intermediate motility sperm in a given population of sperm.

It should be understood that other standardized measures of sperm motility parameters can also be used. Other measures of sperm motility include “velocity” and “linearity” which can be assessed using automatic semen analyzers. In some embodiments, the methods disclosed herein can increase sperm function comprising increase in average path velocity (VAP), straight-line velocity (VSL), curvilinear velocity (VCL), amplitude of lateral head displacement (ALH) and beat cross frequency (BCF) or other movement parameters of the sperm including parameters known to those of skill in the art. Curvilinear velocity (VCL) is the measure of the rate of travel of the centroid of the sperm head over a given time period. Average path velocity (VAP) is the velocity along the average path of the spermatozoon. Straight-line velocity (VSL) is the linear or progressive velocity of the cell. Linearity of forward progression (LIN) is the ratio of VSL to VCL and is expressed as percentage. Amplitude of lateral head displacement (ALH) of the sperm head is calculated from the amplitude of its lateral deviation about the cell's axis of progression or average path. Methods of measuring sperm motility by CASA are well known in the art, see for example, WO2012061578A2. An increase in sperm motility, as contemplated herein, constitutes an increase in the motility of sperm relative to a suitable control sperm.

In some embodiments sperm with increased motility are provided that are the product of a process comprising incubating sperm in energy depletion conditions to potentiate the sperm, followed by providing the potentiated sperm with a first energy source and a second energy source. In some embodiments, the first and second energy sources are provided simultaneously. In some embodiments, the first and second energy sources are provided serially. In some embodiments, the increase in sperm motility can be more than about: 10%, 20%, 30%, 40%, 50%, 60%, 70%, 80%, 90%, or 99% relative to a suitable control sperm. In some embodiments, the increase in sperm motility can be at least 5%, at least 10%, at least 20%, at least 30%, at least 40%, at least 50%, at least 60%, at least 70%, at least 80%, at least 90%, or at least 95%. In some embodiments, the increase in sperm motility can be by a factor of at least 10, at least 100, at least 1,000, at least 10,000. In some embodiments, the sperm motility can be increased by from 10% to 200%, from 25% to 150%, from 50% to 100%, or from 70% to 90%. In some embodiments, the increased sperm function or increased sperm motility can be an increase in percentage of hyperactivated sperm. In some embodiments, the increased sperm function or increase in sperm motility can be an increase in percentage of intermediate motility sperm. In some embodiments, the increased sperm function or increased sperm motility can be an increase in percentage of progressive motility sperm. In some embodiments, the increased sperm function or increased sperm motility can be an increase in percentage of the hyperactivated sperm and intermediate motility sperm. In some embodiments, the level of hyperactivated sperm, progressive motililty sperm, intermediate motility sperm or a combination thereof is increased so that hyperactivated sperm, progressive motililty sperm, intermediate motility sperm or a combination thereof comprise at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. An increase in sperm motility is indicative of increased sperm function.

### Sperm Capacitation

In some embodiments the increased sperm function comprises an increase in sperm capacitation. “Sperm capacitation” refers to the sperm having the ability to undergo acrosomal exocytosis and binding to and penetrating through the zona pellucida of an unfertilized egg. Completion of capacitation is manifested by the ability of sperm to bind to the zona pellucida and to undergo ligand-induced acrosomal reaction. Methods to determine sperm capacitation are known in the art, for example, the most common sperm-zona pellucida binding tests currently utilized are the hemizona assay (or HZA) and a competitive intact-zona binding assay. A hemizona assay measures the ability of sperm to undergo capacitation and bind to an oocyte. Sperm is incubated with dead oocytes which are surrounded by the zona pellucida, an acellular coating of oocytes. Capacitated sperm bind to the zona and the number of sperm binding is counted microscopically. This number correlates with the number of normal capacitated sperm in a sample and with fertility of a sperm sample. For example, see Cross N L et al. Gamete Res. 1986; 15:213-26.

In some embodiments, sperm with increased capacitation are provided that are the product of a process comprising incubating sperm in energy depletion conditions to potentiate the sperm, followed by providing the potentiated sperm with a first energy source and a second energy source simultaneously, or serially. In some embodiments, the increase in sperm capacitation can be more than about: 10%, 20%, 30%, 40%, 50%, 60%, 70%, 80%, 90%, or 99% relative to a suitable control sperm. In some embodiments, the increase in sperm capacitation can be at least 5%, at least 10%, at least 20%, at least 30%, at least 40%, at least 50%, at least 60%, at least 70%, at least 80%, at least 90%, or at least 95%. In some embodiments, the increase in sperm capacitation can be by a factor of at least 10, at least 100, at least 1,000, at least 10,000. In some embodiments, the sperm capacitation can be increased by from 10% to 200%, from 25% to 150%, from 50% to 100%, or from 70% to 90%. In some embodiments, the level of sperm capacitation is increased so that capacitated sperm can comprise at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50%, or more of the total sperm in a preparation. An increase in sperm capacitation is indicative of increased sperm function.

### Fertilizing Ability

In some embodiments, the sperm function comprises ability of the sperm to fertilize an egg. The fertilizing ability of a sperm can be determined, for example, by a sperm penetration assay. The spermatozoa penetration assay (SPA) utilizes the golden hamster egg, which is unusual in that removal of its zona pellucida results in loss of all species specificity to egg penetration. This test is conducted to determine the ability of sperm to penetrate into the oocyte (Rogers et al., Fert. Ster. 32:664, 1979). Briefly, commercially available zona free hamster oocytes can be used (Fertility Technologies, Natick, Mass.). Hamster oocytes are suitable in this assay for sperm of any species. Sperm are incubated for 3 hours with the hamster oocytes. Following incubation, oocytes are stained with acetolacmoid or equivalent stain and the number of sperm penetrating each oocyte is counted microscopically. Another parameter of sperm fertilizing ability is the ability to penetrate cervical mucus. This penetration test can be done either in vitro or in vivo. Briefly, in vitro, a commercial kit containing cervical mucus (Tru-Trax, Fertility Technologies, Natick, Mass.), typically bovine cervical mucus, is prepared. Sperm are placed at one end of the track and the distance that sperm have penetrated into the mucus after a given time period is determined. Alternatively, sperm penetration of mucus may be measured in vivo in women. In an embodiment sperm with increased fertilizing ability are provided that are the product of a process comprising incubating sperm in energy depletion conditions to potentiate the sperm, followed by providing the potentiated sperm with a first energy source and a second energy source simultaneously, or serially. In some embodiments, the increase in fertilizing ability can be more than about: 10%, 20%, 30%, 40%, 50%, 60%, 70%, 80%, 90%, or 99% relative to a suitable control sperm. In some embodiments, the increase in fertilizing ability can be at least 5%, at least 10%, at least 20%, at least 30%, at least 40%, at least 50%, at least 60%, at least 70%, at least 80%, at least 90%, or at least 95%. In some embodiments, the increase in fertilizing ability can be by a factor of at least 10, at least 100, at least 1,000, at least 10,000. In some embodiments, the fertilizing ability can be increased by from 10% to 200%, from 25% to 150%, from 50% to 100%, or from 70% to 90%. In some embodiments, the level of fertilizing ability is increased so that the number of sperm able to fertilize an egg is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. An increase in fertilizing ability is indicative of increased sperm function and increased fertilization.

### Generating Embryos

In some embodiments, sperm function comprises generating an embryo. In some embodiments, the sperm with increased function prepared by methods herein is provided access to an egg to promote fertilization, wherein promoting fertilization can comprise generation of an embryo. In some embodiments, the sperm with increased function prepared by the methods herein is provided access to an egg in vitro, thereby generating the embryo in vitro. In some embodiments, the sperm with increased function prepared by the methods disclosed herein is provided access to an egg in vivo by IUI of the sperm, thereby generating the embryo in vivo. In some embodiments, the sperm which has been incubated under energy deletion conditions and provided with first energy source is inseminated in the reproductive tract of a female subject such that providing the second energy source and providing access to an egg to generate an embryo occurs in vivo. In some embodiments, where the embryo is generated in vitro, the embryo can be cryopreserved for later use or can be further cultured in vitro to enable embryonic development. In some embodiments, the embryo is developed to at least a two cell stage prior to cryopreserving and/or implantation into a female subject. In some embodiments, the embryo is developed to a developmental stage greater than the two-cell stage in vitro prior to further processing. In some embodiments, the embryo is developed to a blastocyst stage in vitro prior to further processing (e.g., cryopreservation or implantation into a female subject to develop into a full offspring). For in vitro incubation and culture of embryos during via assisted reproductive technologies (ART) procedures, a range of suitable media are available, the types and compositions of which are well known to those of skill in the art. Preferably the culture medium contains at least water, salts, nutrients, essential amino acids, vitamins and hormones, and may also include one or more growth factors. A variety of suitable culture media is commercially available, for example Earle's media, Ham's F10 media and human tubal fluid (HTF) media. The present disclosure also contemplates the co-culture in vitro of embryos on a layer of ‘feeder cells’ by methods known in the art. Appropriate ‘feeder cells’ for co-culture may include, for example, bovine oviductal cells or human tubal epithelial cells.

Those of skill in the art will appreciate that the advantages offered by the sperm with increased function prepared by the methods disclosed herein are not limited to increasing fertilization. Rather the methods and preparation of the present invention are equally applicable as treatment to promote fertilization, whether the embryos are produced in vitro via assisted reproductive technologies (ART) or in the reproductive tract of the animal. The methods of the present invention are applicable to improving fertilization, embryo viability, embryo implantation and pregnancy rates in assisted or otherwise unassisted pregnancies. Embodiments of the present disclosure also provide for methods of increasing the fertilizing ability of sperm in male animals.

In the context of this specification, the terms “embryo with increased viability” and “embryo with longer viability” mean an increase or enhancement in the likelihood of survival of an embryo(s) which has been generated by the mammalian sperm of the methods and preparation disclosed herein, for example, a mammalian sperm with one or more increased sperm function, compared to the likelihood of survival of an embryo(s) which has been generated by a suitable control sperm. In some embodiments, the embryo is generated by an assisted reproductive technology e.g., IVF or ICSI. In some embodiments, the embryo is generated in vivo in the reproductive tract of a female mammalian subject by artificial insemination.

For the purposes of the present disclosure, embryo viability may be reflected in a number of indicators. For example, increased embryo viability may result in increased embryo implantation rates following fertilization, decreased pre- and post-implantation embryo lethality, increased clinical pregnancy rates or increased birth rates. The present disclosure therefore also relates to methods of preventing apoptosis or retarded development in embryos and to methods of increasing pregnancy rates in animals. The embryo viability can refer to viability of an embryo in vitro or in vivo.

In some embodiments, sperm with ability to generate an embryo with increased viability is provided that are the product of a process comprising incubating sperm in an energy depletion conditions to potentiate the sperm, followed by providing the potentiated sperm with a first energy source and a second energy source simultaneously, or serially. In some embodiments, providing the sperm with increased function access to an egg promotes fertilization. In some embodiments, promoting fertilization comprises generation of an embryo(s) with increased viability. In some embodiments, the increase in viability of embryo generated by the sperm prepared by methods herein upon access to an egg can be more than about: 10%, 20%, 30%, 40%, 50%, 60%, 70%, 80%, 90%, or 99% relative to an embryo generated by a suitable control sperm. In some embodiments, the increase in embryo viability can be at least 5%, at least 10%, at least 20%, at least 30%, at least 40%, at least 50%, at least 60%, at least 70%, at least 80%, at least 90%, or at least 95%. In some embodiments, the increase in embryo viability can be by a factor of at least 10, at least 100, at least 1,000, at least 10,000. In some embodiments, the embryo viability can be increased by from 10% to 200%, from 25% to 150%, from 50% to 100%, or from 70% to 90%. In some embodiments, the level of sperms that can generate an embryo with increased viability is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. Generation of an embryo with increased viability is indicative of increased sperm function and/or increased fertilization.

Typically the cleavage stage of embryo occurs during the first three days of culture. The in vitro generated embryo is transferred to a female subject by embryo transfer. “Embryo transfer” is the procedure in which one or more embryos and/or blastocysts are placed into the uterus or fallopian tubes. In the traditional IVF process, embryos are transferred to the uterine cavity two days after fertilization when each embryo is at the four (4) cell stage or three days after fertilization when the embryo is at the eight (8) cell stage or 5 days after fertilization when the embryo is at the blastocyst stage. It has been recognized that it may be desirable to use embryos at the blastocyst stage when reached at day five to seven of culture. The present disclosure allows for embryo transfer at any time along the spectrum of embryo/blastocyst development. Through visual observation, such as by with the use of microscopy, blastocysts or embryos are considered ready to be transferred to the uterus when the blastoceol cavity is clearly evident and comprises greater than 50% of the volume of the embryo. In an in vivo environment, this stage would normally be achieved four to five days after fertilization, soon after the embryo has traversed the fallopian tube and arrives in the uterus. Embryonic developmental stage can be determined by visual observation of the embryo using microscopy (for example, Nikon Eclipse TE 2000-S microscope), the embryo will display certain determined physical or morphological features simultaneously before it is implanted into the uterus. The state of blastocyst maturity will be determined to be the range II AB-VI AA according to classification of Gardner et al., 1998.

The methods disclosed herein result in generation of embryos with increased rate of progressing to 2-cell developmental stage, blastocyst developmental stage, or development to an offspring and live birth. In some embodiments, sperm which can generate an embryo with ability to develop through normal developmental stages (e.g., 2 cell stage, blastocyst stage, development into an offspring and live birth) is provided that are the product of a process comprising incubating sperm in an energy depletion conditions to potentiate the sperm, followed by providing the potentiated sperm with a first energy source and a second energy source simultaneously, or serially. In some embodiments, providing the sperm with increased function access to an egg promotes fertilization. In some embodiments, promoting fertilization comprises generation of embryos with increased ability to develop through normal developmental stages (e.g., 2 cell stage, blastocyst stage, development into an offspring and live birth). In some embodiments, increase in rate of an embryo progressing through normal developmental stages, generated by the sperm prepared by methods can be more than about: 10%, 20%, 30%, 40%, 50%, 60%, 70%, 80%, 90%, or 99% relative to an embryo generated by suitable control sperm. In some embodiments, the increase in rate of an embryo progressing through normal developmental stages can be at least 5%, at least 10%, at least 20%, at least 30%, at least 40%, at least 50%, at least 60%, at least 70%, at least 80%, at least 90%, or at least 95%. In some embodiments, the increase in rate of an embryo progressing through normal developmental stages can be by a factor of at least 10, at least 100, at least 1,000, at least 10,000. In some embodiments, the rate of embryo progressing through normal developmental stages can be increased by from 10% to 200%, from 25% to 150%, from 50% to 100%, or from 70% to 90%. In some embodiments, the level of sperms that can generate an embryo with ability to progress through normal developmental stages is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. Generation of an embryo with ability to progress through one or more normal developmental stages is indicative of increased sperm function and/or increased fertilization.

In vivo, an embryo attaches or implants to a wall of the uterus, creates a placenta, and develops into a fetal offspring during gestation until childbirth. Testing to determine whether one or more embryos have implanted into the endometrium, i.e, whether the procedure has resulted in successful pregnancy inception, is performed two weeks after transfer using blood tests on b-hCG (human chorionic gonadotropin), for example, and other techniques commonly known in the art. U.S. Pat. No. 4,315,908 to Zer et al. sets forth a method for detecting hCG in the urine by radioimmunoassay. U.S. Pat. No. 8,163,508 to O'Connor et al. provides a method and a kit for predicting pregnancy in a subject by hCG method by determining the amount of an early pregnancy associated isoform of hCG in a sample. Such methods of diagnosis and others are useful within the scope of the disclosure.

In some embodiments, sperm with ability to generate an embryo with improved implantation rate or improved rate of pregnancy is provided that are the product of a process comprising incubating sperm in an energy depletion conditions to potentiate the sperm, followed by providing the potentiated sperm with a first energy source and a second energy source simultaneously, or serially. In some embodiments, providing the sperm with increased function access to an egg promotes fertilization. In some embodiments, promoting fertilization comprises generation of an embryo with improved implantation rate or improved rate of pregnancy. In some embodiments, the increase in implantation rate of an embryo generated by the sperm prepared by methods herein or pregnancy rate upon implantation of an embryo can be more than about: 10%, 20%, 30%, 40%, 50%, 60%, 70%, 80%, 90%, or 99% relative to an embryo generated by a suitable control sperm. In some embodiments, the increase in an embryo implantation rate or pregnancy rate can be at least 5%, at least 10%, at least 20%, at least 30%, at least 40%, at least 50%, at least 60%, at least 70%, at least 80%, at least 90%, or at least 95%. In some embodiments, the increase in rate of embryo implantation or rate of pregnancy can be by a factor of at least 10, at least 100, at least 1,000, at least 10,000. In some embodiments, the embryo implantation or pregnancy rate can be increased by from 10% to 200%, from 25% to 150%, from 50% to 100%, or from 70% to 90%. In some embodiments, the level of sperms that can generate an embryo with increased implantation rate or improved pregnancy rate is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. Generation of embryos with improved implantation (i.e., increased rate of implantation) or increased pregnancy rate upon implantation is indicative of increased sperm function and/or increased fertilization.

### Autophagy

In some embodiments, the increased sperm function comprises an increase in autophagy. Methods to determine an increase in autophagy are known in the art. For example, an increase in autophagy can be determined by increase in one or more of autophagy marker proteins. The detection of increase in marker protein can be done by conventional methods such as immunoblotting. Non-limiting examples of autophagy marker proteins include, Atg 5, Atg 16, p62, LC3-II, AMPK, m-TOR and Beclin 1. LC3-II has been widely used to study autophagy and it has been considered as an autophagosomal marker in mammals. A ratio of LC3-II/LC3-I can be used as a determinant of increase in autophagy. An increase in levels of one or more autophagy marker proteins (e.g., Atg 5, Atg 16, p62 and LC3-II, AMPK, m-TOR and Beclin 1), and/or an increase in ratio of LC3-II/LC3-I can be indicative of increase in sperm function. In some embodiments, an increase in autophagy can be indicated by a reduction in cellular RNA levels (such as small non-coding RNAs, including microRNA).

In some embodiments, sperm with increased autophagy are provided that are the product of a process comprising incubating sperm in energy depletion conditions to potentiate the sperm, followed by providing the potentiated sperm with a first energy source and a second energy source simultaneously, or serially. In some embodiments, the increase in autophagy can be more than about: 10%, 20%, 30%, 40%, 50%, 60%, 70%, 80%, 90%, or 99% relative to a suitable control sperm. In some embodiments, the increase in sperm autophagy can be at least 5%, at least 10%, at least 20%, at least 30%, at least 40%, at least 50%, at least 60%, at least 70%, at least 80%, at least 90%, or at least 95%. In some embodiments, the increase in sperm autophagy can be by a factor of at least 10, at least 100, at least 1,000, at least 10,000. In some embodiments, the sperm autophagy can be increased by from 10% to 200%, from 25% to 150%, from 50% to 100%, or from 70% to 90%. In some embodiments, the level of sperm autophagy is increased so that sperm with increased autophagy can comprise at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. An increase in sperm autophagy is indicative of increased sperm function.

### Starvation

“Energy depletion” means suppressing or restricting the energetic output of a cell whether by depletion, reduction (below an effective amount), or removal of such energy sources or inhibition of enzymatic or import machinery. In some embodiments one or more of glycolysis, gluconeogenesis, Kreb's cycle, or oxidative phosphorylation are inhibited in the energy depletion and, in particular embodiments, the energy depletion includes glycolytic energy depletion. Exemplary conditions of glycolytic energy depletion include removing substantially all of glycolytically-liable sugar, such as glucose (other embodiments can include, mannose, fructose, dextrose, sucrose, and combinations thereof, including combinations with glucose), in the sperm's medium or reducing the concentration of glycolytically-liable sugar, or using inhibitors of glycolysis, gluconeogenesis, or importers of glycolytically-liable sugars. As glucose is a primary energy source of sperm, in preferred embodiments, the energy depletion is glucose energy depletion (including starvation), which further entails depletion (including starvation) of gluconeogenesis substrates (including, e.g., pyruvate or, in some embodiments lactate, which can be converte to pyruvate by lactate dehydrogenase), and Kreb's cycle substrates (acetyl CoA, citrate, isocitrate, alpha-ketoglutarate, succinyl-CoA, succinate, fumarate, malate, and oxaloacetate).

In some embodiments, the energy depletion comprises a low glucose concentration, e.g., less than about: 0.5, 0.4, 0.3, 0.2, 0.1, 0.09, 0.08, 0.07, 0.06, 0.05, 0.04, 0.03, mM glucose, or less, such as less than about: 0.02 or 0.01 mM, e.g., less than about 0.01 mM. In some embodiments the energy depletion means a substantially glucose-free condition. The invention provides methods entailing staged provision of effective amounts of first and second energy sources and the skilled artisan will appreciate that in some embodiments encompassed within the invention, sub-effective amounts of a glycolytic energy source are an energy depletion and, for example, the foregoing low glucose concentrations can be employed in such embodiments as an energy depletion.

In some embodiments, the energy depletion comprises a low pyruvate concentration, e.g., less than about: 0.15, 0.10, 0.09, 0.08, 0.07, 0.06, 0.05, 0.04, 0.03, 0.02, 0.01, 0.005, 0.003, 0.002 mM, or less. In some embodiments the energy depletion means a substantially pyruvate-free condition. As noted above and exemplified with glucose for a glycolytic energy source, the skilled artisan will also appreciate that in some embodiments encompassed within the invention sub-effective amounts of a gluconeogenesis substrate are an energy depletion and, for example, the foregoing low pyruvate concentrations can be employed in such embodiments as an energy depletion

In some particular embodiments, the energy depletion comprises a condition substantially free of carbon sources, such as low glucose concentration and low pyruvate concentration, e.g., a substantially glucose-free and substantially pyruvate-free condition.

In some embodiments, the energy depletion is for at least about: 10, 20, 30, 40, 50, 60 minutes, e.g., at least about: 30, 40, 45, 50, 55, 60, 90, 120, 150, or 180 minutes or 1, 1.5, 2, 2.5, 3, 3.5, 4, 4.5, 5, 5.5, 6, 6.5, 7, 7.5, 8, 8.5, 9, 9.5, or 10 hours.

Energy depletion consonant with the invention potentiates the sperm. “Potentiate” or “potentiating” sperm means to condition sperm such that, upon a suitable induction, e.g., removing or reversing the energy depletion and, e.g., incubating the sperm in capacitation conditions or staged energy reintroduction, the sperm rapidly recover motility, such as one or more of: an increased proportion of hyperactivated, intermediate, or progressive motility sperm (or an increased proportion of a combination of two (such as hyperactivated and intermediate) or all three), and/or increased curvilinear velocities.

### Staged Energy Reintroduction

Following energy depletion sufficient to potentiate the sperm, an effective amount of a first and then an effective amount of a second energy source is provided to the potentiated sperm. In some embodiments, the time between providing an effective amount of a first energy source after potentiating the sperm and providing an effective amount of a second energy source is at least about: 1, 2, 3, 4, 5, 10, 15, 20, 25, 30, 35, 40, 45, 50, 55, or 60 minutes, e.g., at least 5, 6, 7, 8, 9, 10, 11, 12, 13, 14, 15, 16, 17, 18, 19, or 20 minutes, e.g., at least between about: 5-15 minutes. In some embodiments, the time between providing an effective amount of a first energy source after potentiating the sperm and providing an effective amount of a second energy source is longer, such as at least 2, 3, 4, or 5 hours, or more.

In some embodiments, the gluconeogenesis substrate is pyruvate, e.g., at a concentration of about: 0.15-0.66 mM, e.g., about 0.20-0.50 mM, such as about 0.25-0.40 mM, or about 0.30 mM. The forgoing concentrations are exemplary effective amounts of a gluconeogenesis substrate, for example, when provided as either a first or second energy source in the methods provided by the invention. The skilled artisan will recognize other effective amounts of gluconeogenesis substrates by virtue of their ability to increase sperm function consonant with the teachings of the invention. In some embodiments, the first energy source is a gluconeogenesis substrate, such as pyruvate. In some embodiments, the second energy source is a gluconeogenesis substrate, such as pyruvate.

In some embodiments, the glycolytic energy source is glucose, e.g., at a concentration of about: 0.6 mM-10.0 mM, 1.0-7.0 mM, 2.5-7.0 mM, 3.5-6.5 mM or 5 mM, e.g., at least about: 1, 2, 3, or 4 mM. The forgoing concentrations are exemplary effective amounts of a glycolytic energy source, for example, when provided as either a first or second energy source in the methods provided by the invention. The skilled artisan will recognize other effective amounts of glycolytic energy sources by virtue of their ability to increase sperm function consonant with the teachings of the invention. In some embodiments, the first energy source is a glycolytic energy source, such as glucose. In some embodiments the second energy source is a glycolytic energy source, such as glucose. In some embodiments, the first energy source is a glycolytic energy source, such as glucose, while the second energy source is a gluconeogenesis substrate, such as pyruvate.

An additional condition regulated in some embodiments of the methods provided by the invention is the osmolarity (mOsm) or osmolality (mOsm/kg). In some embodiments, the method is performed at an osmolarity (or osmolality) ranging from between about: 200-280 mOsm (mOsm/kg), e.g., between about: 220-260, 225-255, 230-250 mOsm (mOsm/kg) during energy depletion, optionally, wherein upon addition of the first or second energy source, the osmolarity (or osmolality) is increased to at least about: 270, 275, 280, 285, 290, or 295 mOsm (mOsm/kg).

Gluconeogenesis substrate suitable for use in the methods of the present disclosure include, but are not limited to, pyruvate, lactate, succinate, citrate, fumarate, malate, aspartate, glycerol, acetyl CoA, isocitrate, alpha-ketoglutarate, succinyl-CoA, oxaloacetate; or a physiologically acceptable derivative, salt, ester, polymer or alpha-keto analogue of the gluconeogenesis substrate. Any gluconeogenic amino acid, or a physiologically acceptable derivative, salt, ester, or polymer, or alpha-keto analogue thereof is also suitable as a gluconeogenesis substrate. Non-limiting examples of gluconeogenic amino acids include alanine, arginine, asparagine, cystine, glutamine, glycine, histidine, hydroxyproline, methionine, proline, serine, threonine and valine. Non-limiting examples of pharmaceutically acceptable salts of pyruvate are lithium pyruvate, sodium pyruvate, potassium pyruvate, magnesium pyruvate, calcium pyruvate, and zinc pyruvate. In some embodiments, the pyruvate is sodium pyruvate. Non-limiting examples of salts of lactate include sodium lactate, potassium lactate, magnesium lactate, calcium lactate, zinc lactate, and manganese lactate. The gluconeogenesis substrate of the methods disclosed herein can be any one of the gluconeogenesis substrates listed above.

Glycolytic energy source suitable for use in the methods of the present disclosure include but are not limited to carbon sources for glycolysis. Non-limiting examples of glycolytic energy source useful in the methods disclosed herein include monosaccharides (such as fructose, glucose, galactose and mannose) and disaccharides (sucrose, lactose, maltose, and trehalose), as well as polysaccharides, galactose, oligosaccharides, polymers thereof.

In some embodiments of the methods provided by the invention, additional components are provided to the sperm. For example, other components upstream and downstream of glycolysis such as NADH, NAD+, citrate, AMP, ADP, or a combination thereof are added in combination with at least the first energy source or the second energy source.

Some embodiments of the methods provided by the invention include assessment of the sperm. For example, in some embodiments, the methods include one or more quantitative assessments of sperm motility, e.g., by CASA, and/or measuring sperm quality, such as DNA fragmentation (e.g., by TUNEL), lipid peroxidation, reactive oxygen species, or a combination thereof.

The methods provided by the invention achieve increased sperm function. In some embodiments, relative to a suitable control sperm. In some embodiments the suitable control is sperm in standard capacitation medium (C-HTF), without a starvation step, while in some embodiments, the suitable control is sperm in standard capacitation medium (C-HTF) following a three hour starvation—e.g., starvation and reintroduction of effective amounts of energy sources without staging reintroduction of the energy sources.

### Mammalian Sperm

The methods disclosed herein comprise increasing one or more functions of a sperm. The present disclosure also relates to promoting fertilization. Preparations of sperm with increased function are also provided. As used herein, the sperm can be from a vertebrate, preferably a mammal. Accordingly, the sperm of the present disclosure can be a mammalian sperm.

Mammals include, without limitation, humans, primates, rodents, wild or domesticated animals, including feral animals, farm animals, sport animals, and pets. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The mammalian sperm can be from a non-human mammal including, an ungulate, such as an even-toed ungulate (e.g., pigs, peccaries, hippopotamuses, camels, llamas, chevrotains (mouse deer), deer, giraffes, pronghorn, antelopes, goat-antelopes (which include sheep, goats and others), or cattle) or an odd-toed ungulate (e.g., horse, tapirs, and rhinoceroses), a non-human primate (e.g., a monkey, chimpanzees, cynomologous monkeys, spider monkeys, and macaques, e.g., Rhesus), a Canidae (e.g., a dog) or a cat. The mammalian sperm can be from a member of the Laurasiatheria superorder. The Laurasiatheria superorder can include a group of mammals as described in Waddell et al., Towards Resolving the Interordinal Relationships of Placental Mammals. Systematic Biology 48 (1): 1-5 (1999). The Members of the Laurasiatheria superorder can include Eulipotyphla (hedgehogs, shrews, and moles), Perissodactyla (rhinoceroses, horses, and tapirs), Carnivora (carnivores), Cetartiodactyla (artiodactyls and cetaceans), Chiroptera (bats), and Pholidota (pangolins). A member of Laurasiatheria superorder can be an ungulate, e.g., an odd-toed ungulate or even-toed ungulate. An ungulate can be a pig. The mammalian sperm can be from a member of Carnivora, such as a cat, or a dog. In some embodiments, the mammalian sperm is a human, non-human primate, porcine, bovine, equine, ovine, canine, feline, or murine sperm. In some embodiments, the mammalian sperm is a human sperm.

In some embodiments, the mammalian sperm is from a healthy male mammal. In some embodiments, the mammalian sperm is from a male suffering from sperm dysfunction, for example, low sperm count, reduced motility of sperm, and abnormal morphology of sperm. In some embodiments, the mammalian sperm can be from a subfertile male or an oligospermic male. The mammalian sperm can be from a male suffering from, for example, oligospermia, Teratozoospermia, Asthenozoospermia, or Oligoasthenoteratozoospermia. Oligospermia refers to a condition characterized by sperm concentration of <20 million/ml. Asthenozoospermia refers to a condition characterized by reduced sperm motility. Teratozoospermia refers to a condition characterized by presence of sperm with abnormal morphology. Oligoasthenoteratozoospermia refers to a condition that includes oligozoospermia (low number of sperm), asthenozoospermia (poor sperm movement), and teratozoospermia (abnormal sperm shape). In some embodiments, the sperm is obtained from a subfertile human male or an oligospermic human male, e.g., having a sperm count below about: 20, 19, 18, 18, 16, 15, 14, 13, 12, 11, 10, 9, 8, 7, 6, 5, 4, 3, 2 million sperm per milliliter, e.g., less than 15 million sperm per milliliter.

In some embodiments, the sperm are enriched (or isolated) from semen prior to energy depletion. Any method of sperm enrichment or isolation can be used consonant with the invention, including density gradient centrifugation, swim up, microfluidics, or a combination thereof.

Sperm may be used in the methods provided by the invention either fresh or from a preserved stock. For example, in some embodiments, prior to treatment, the sperm are recovered from cryogenic storage. In some embodiments, prior to the treatment, the sperm are recovered from non-cryogenic storage. In some embodiments, prior to treatment, the sperm are provided in a preservation medium. In some embodiments, the sperm in preservation medium is stored in cryogenic conditions prior to incubating under energy depletion conditions. In some conditions, the sperm in preservation medium is stored in non-cryogenic conditions prior to incubating under energy depletion conditions.

In some embodiments, the preservation medium is a buffered solution comprising a slightly acidic pH and having an osmolality of between about: 300 and 400 mOsm/kg, e.g., between about: 300-380, 320-370, 330-370, 340-360, e.g., about: 320, 330, 340, 350, 360, 370, or 380, e.g., about 350 mOsm/kg. In some embodiments, the osmolality of the preservation media provided by the invention is between about: 320-340 mOsm/kg. A “slightly acidic” pH means less than 7, but more than 5. In some embodiments, a slightly acidic pH is between about: 6 and 7, e.g., greater than, or about: 6.1, 6.2, 6.3, 6.4, 6.5, 6.7, 6.8, 6.9 and less than 7.0 (such as 6.99), e.g., between about: 6.5 and 6.99, such as between about: 6.7-6.9, e.g., about 6.8. In some embodiments, the preservation medium is a buffered solution having a pH of between about: 6.7 and 6.9, comprising (or consisting essentially of) a zwitterionic buffer, glucose at a concentration of between about: 0.25-0.35 M, an antibiotic, osmolality of between about 340-360 mOsm/kg, BSA or HSA at a concentration of about: 2-4% (W/V), wherein sperm stored in the preservation medium for up to 12 days at 4° C. maintain at least 60% motile sperm upon transfer to capacitation medium, relative to suitable control sperm; optionally wherein the medium does not contain egg yolk.

Different quantities of sperm can be used in the methods provided by the invention, including fractions of a single ejaculate or a whole ejaculate. In some embodiments, the sperm are pooled from two or more ejaculates (e.g., 2, 3, 4, 5, 6, or more ejaculates).

### Preservation Media

In one aspect, the invention provides sperm preservation media. These preservation media provided by the invention can advantageously be incorporated for use in methods provided by the invention (e.g., inducing increased sperm function, promoting fertilization, producing offspring with improved fitness etc.), which methods can, in some embodiments, be performed using the various kits provided by the invention to then, in certain embodiments, produce the sperm preparations provided by the invention, and/or in additional methods provided by the invention, such as methods of fertilization, including methods of assisted reproduction. These preservation media provided by the invention can advantageously be incorporated in the kits provided by the invention, which kits can, in some embodiments, be useful for performing the various methods provided by the invention to then, in certain embodiments, produce the sperm preparations provided by the invention, and/or in additional methods provided by the invention, such as inducing increased sperm function, promoting fertilization, producing offspring with improved fitness, methods of fertilization, including methods of assisted reproduction. In some embodiments, the sperm are recovered from the preservation media prior to performing the methods disclosed herein. In some embodiments, the recovery of the sperm from the preservation media comprises enrichment, washing or diluting the sperm sample, for example, using the kits of components thereof as disclosed herein. In some embodiments, the sperm or preparations of sperm of the disclosure can be stored in the preservation medium after performing the methods described herein, for example, which methods are performed using the kits disclosed herein.

The preservation media provided by the invention comprise a buffered solution comprising a slightly acidic pH and having an osmolality of between about: 300 and 400 mOsm/kg, e.g., between about: 300-380, 320-370, 330-370, 340-360, e.g., about: 320, 330, 340, 350, 360, 370, or 380, e.g., about 350 mOsm/kg. In certain other embodiments, the osmolality of the preservation media provided by the invention is between about: 320-340 mOsm/kg. A “slightly acidic” pH means less than 7, but more than 5. In some embodiments, a slightly acidic pH is between about: 6 and 7, e.g., greater than, or about: 6.1, 6.2, 6.3, 6.4, 6.5, 6.7, 6.8, 6.9 and less than 7.0 (such as 6.99), e.g., between about: 6.5 and 6.99, such as between about: 6.7-6.9, e.g., about 6.8. These preservation media, including the embodiments described below, are referred to as “preservation medi(um/a) provided by the invention” or “sperm preservation medi(um/a) provided by the invention” and the like.

“Sperm preservation” refers to maintaining the viability and function of sperm over time as assessed by, for example, the ability to recover motility in conditions known to induce capacitation of sperm. The preservation media provided by the invention and methods comprising preserving sperm in said preservation medium provided by the invention advantageously may do one or more of: preserve such sperm function, limit damage and degradation of the sperm, or a combination thereof. In certain embodiments, capacitation is induced and measured using techniques known in the art, such as disclosed in Molina et al. 2018 (doi.org/10.3389/fcell.2018.00072)—briefly, capacitation is performed for 4 hours at 37° C. and 5% CO₂ in modified human tubal fluid (mHTF) medium (HEPES 21 mM, NaCl 97.8 mM, KCl 4.7 mM, KH₂PO₄ 0.37 mM, MgSO₄ 0.2 mM, CaCl₂ 2 mM, glucose 2.78 mM, pyruvate 0.33 mM, lactate 21.4 mM) supplemented with 5 mg/ml human serum albumin and 25 mM sodium bicarbonate. In some embodiments, sperm preservation entails inducing a quiescent state rapidly (e.g., within about: 1, 2, 3, 4, 8, 12, 16, 20, or 24 hours upon storage at 4° C., in some embodiments, within about 24 hours upon storage at 4° C., while retaining the ability to quickly (e.g., within about: 1, 2, 3, or 4 hours, or less, in some embodiments about: 30, 40, 50, or 60 minutes) recover motile sperm in capacitation conditions (supra), e.g., 60, 65, 70, 75, 80, 85, 90, 95%, or more, e.g., 96, 97, 98, 99% of the number of pre-preservation motile sperm, that is, relative to control. Thus, in some embodiments, following a rapid induction of quiescence, the preservation media provided by the invention provide a high percentage (e.g., 60, 65, 70, 75, 80, 85, 90, 95%, or more) of the number of motile sperm, relative to a control sample, taken after enrichment of sperm but before quiescence, upon incubation at 37° C. and 5% CO₂ in modified human tubal fluid (mHTF) medium supplemented with 5 mg/ml human serum albumin and 25 mM sodium bicarbonate.

In certain embodiments, the preservation media provided by the invention lack (either completely or a significant amount of) one or more (i.e., 1, 2, 3, 4, or all 5) of: egg yolk, added electrolytes (other than those attributed to buffer, carbon source, optionally a serum albumin, or optionally an antibiotic), glycerol, lecithin, or dextrose. In other embodiments, the preservation medium may comprise a cryoprotectant, such as glycerol. In certain embodiments, the sperm preservation media provided by the invention can also include additional components further comprising, osmolytes (including ionic components, such as calcium), lipids, viscosity control agents, sterols, antioxidants (such as trehalose), an anti-inflammatory agent (e.g., doxycycline), and combinations of the foregoing.

In some embodiments, the sperm preservation medium provided by the invention includes a carbon source, such as glucose, fructose, mannose, sucrose, or a combination thereof (e.g., 1, 2, 3, or all 4). In some embodiments, the carbon source includes glucose. In certain embodiments, glucose is the primary (>50%) or substantially only carbon source. In certain embodiments, glucose is present in the preservation media provided by the invention at a concentration of between about: 0.1-0.7 M, such as between about 0.2-0.5 M, e.g., between about: 0.25-0.36, 0.25-0.35, 0.30-0.36, or 0.3-0.33M, or about: 0.20, 0.25, 0.26, 0.27, 0.28, 0.29, 0.30, 0.31, 0.32, 0.33, 0.34, 0.35, 0.36, 0.37 M. In some embodiments, the carbon source, such as glucose, in the preservation media provided by the invention is the primary (e.g., >50%, such as 55, 60, 65, 70, 75, 80, 85, 90, 95, 96, 97, 98%, or more) osmolyte, but concentration of the primary osmolyte can be readily adjusted to maintain an osmolality consonant with the invention, e.g., 300-400 mOsm/kg.

The sperm preservation provided by the invention includes a buffer to help regulate the slightly acidic condition. In some embodiments the buffer is not a bicarbonate buffer. In certain embodiments, the buffer is a zwitterionic buffer, such as 4-(2-hydroxyethyl)-1-piperazineethanesulfonic acid (HEPES), 3-(N-morpholino)propanesulfonic acid (MOPS), or a combination thereof. The concentration of the buffer component(s) can be varied, e.g., between about: 1 and 100 mM, e.g., 1 and 50 mM, 1 and 40 mM, 1 and 30 mM, 1 and 20 mM, 5-15 mM; e.g., about: 2, 3, 4, 5, 6, 7, 8, 9, 10, 11, 12, 13, 14, 15, 16, 17, 18, 19, or 20 mM, e.g., about 10 mM. In certain particular embodiments, the buffer is 4-(2-hydroxyethyl)-1-piperazineethanesulfonic acid (HEPES), 3-(N-morpholino)propanesulfonic acid (MOPS), or a combination thereof. In some embodiments, the buffer is HEPES. In other embodiments, it is MOPS. In still other embodiments, the buffer is a mixture of HEPES and MOPS. In such embodiments HEPES and MOPS are used in a proportion of 10:90, 20:80, 30:70, 40:60, 50:50, 60:40, 70:30, 80:20, or 90:10; e.g., in a 10 mM buffer, in a 50:50 mix of HEPES and MOPS, each of HEPES and MOPS is present at 5 mM.

The sperm preservation media provided by the invention, in some embodiments, includes an antibiotic. A variety of antibiotics are suitable for use in the invention and one exemplary class is an aminoglycoside, such as gentamicin. In these particular embodiments, the gentamicin may be present in the preservation media provided by the invention at a concentration of between about: 5 and 20 μg/ml, e.g., about 10 μg/ml.

An additional component of the preservation media provided by the invention, in some embodiments, is a serum albumin. While multiple sources of serum albumin are useful consonant with the invention, in some embodiments, the serum albumin is bovine serum albumin (BSA), human serum albumin (HSA), or a combination thereof. When serum albumin is present, in certain embodiments, the serum albumin (e.g., BSA, HSA, or a combination) is present at a concentration of about: 1.5-4.5% (W/V), e.g., about: 2-4%, about: 2.5-3.5%, or about 3%.

In certain embodiments, the sperm preservation media provided by the invention includes a zwitterionic buffer (as noted supra) and pH of between about: 6.6 and 6.9, glucose at a concentration of between about: 0.25-0.36 M, and osmolality of between about: 320-380 mOsm/kg, wherein, in particular embodiments, the medium does not comprise egg yolk. Consonant with the teachings herein, these embodiments can also further include an antibiotic, such as gentamicin. In some embodiments, the preservation media have a pH of about 6.8 (e.g., using HEPES, MOPS, or a combination thereof), a glucose concentration of about 0.330 mM, osmolality of about 350 mOsm/kg, and wherein the serum albumin is BSA or HSA, optionally wherein the BSA or HSA is present at a concentration of about: 2-4% (W/V).

As noted above, without being bound by theory, the sperm preservation media provided by the invention advantageously preserves a high level of sperm function, while minimizing sperm damage. In certain embodiments of the sperm preservation media provided by the invention, sperm stored in the preservation medium for up to 4, 5, 6, 7, 8, 9, 10, 11, 12 days, or more, at about 4° C. maintain at least about: 40, 45, 50, 55, 60, 65, 70, 75, 80%, or more, motile sperm following incubation in capacitation conditions, relative to suitable controls, such as the total number of motile sperm present in the sample prior to preservation and refrigeration. For example, in certain embodiments, sperm stored in the preservation medium provided by the invention for 7 days at about 4° C., have at least about: 40, 45, 50, 55, 60, 65, 70, 75, 80%, or more, motile sperm upon transfer to capacitation medium, relative to suitable control sperm. In some embodiments, sperm stored in the preservation medium provided by the invention for 4, 5, 6, 7, 8, 9, 10, 11, 12 days, or more, at about 4° C., have at least about 75% motile sperm upon transfer to capacitation medium, relative to suitable control sperm. In some embodiments, sperm stored in the preservation medium provided by the invention for 7 days at about 4° C. have a percent motile sperm that is no less than 1%, 2, 5%, 10%, 15%, or 20% less than the percent motile sperm before storage in the preservation medium.

In addition to the retained function of sperm, in certain embodiments, quality of the sperm stored in preservation media provided by the invention is evident after seven days of incubation by, e.g., reduced TUNEL staining, reduced lipid peroxidation, and reduced reactive oxygen species, or a combination thereof, relative to control refrigeration or cryopreservation samples. For example, in some embodiments, sperm stored in the preservation media provided by the invention exhibit reduced TUNEL staining of at least: 30, 40, 50, 55, 60, 65, 70, 80, 85, 90, 95%, or more, relative to cryogenically stored cells, e.g., after seven days in storage in either medium provided by the invention or cryopreservation medium (TYB with gentamicin and 12% glycerol) and thaw/recovery. In certain embodiments, the TUNEL staining is performed using the methods in Simon et al. (Hum Reprod. 2014 May; 29(5):904-17; additional assays can be used according to Gorczyca et al. Int J Oncol 1(6): 639-48 (1992). In certain embodiments, sperm stored in the preservation media provided by the invention exhibit reduced lipid peroxidation as measured by flow cytometry using BODIPY C11 of at least: 30, 40, 50, 55, 60, 65, 70, 80, 85, 90, 95%, or more, relative to cryogenically stored cells, e.g., after seven days in storage in either medium provided by the invention or cryopreservation medium (TYB with gentamicin and 12% glycerol) and thaw/recovery. In certain embodiments, lipid peroxidation is evaluated by the method of Naguib, Anal Biochem. 265(2):290-8 (1998) or Pap et al., FEBS Lett. 453(3):278-82 (1999). In certain embodiments, sperm stored in the preservation media provided by the invention exhibit reduced reactive oxygen species of at least: 30, 40, 50, 55, 60, 65, 70, 80, 85, 90, 95%, or more, relative to cryogenically stored cells, e.g., after seven days in storage in either medium provided by the invention or cryopreservation medium (TYB with gentamicin and 12% glycerol) and thaw/recovery.

In certain embodiments, any of the media provided by the invention are provided as a sterile formulation. Such sterile formulations may be in a sealed sterile container. In certain embodiments, the sterile formulation is a liquid formulation. In other embodiments, the medium is lyophilized. As will be appreciated by the skilled artisan, lyophilized formulations are substantially free of water but are formulated such that, upon reconstitution, e.g., with sterile, distilled water, the formulation is a medium provided by the invention, specifically with the pH, osmolality, and concentration of other components consonant with the invention.

Accordingly, in certain illustrative embodiments, the invention provides a sperm preservation medium that is a sterile liquid having a pH of between about: 6.7 and 6.9, comprising (or consisting essentially of) a zwitterionic buffer, glucose at a concentration of between about: 0.25-0.35 M, osmolality between about: 340-360 mOsm/kg, an antibiotic, BSA or HSA at a concentration of about: 2-4% (W/V), where sperm stored in the preservation medium for up to 14 days at 4° C. maintains at least 60% of motile sperm upon transfer to capacitation conditions, relative to suitable controls, the number of motile sperm at time 0. While illustrated here in a particular embodiment, components of such particular preservation media provided by the invention can be varied as illustrated above, including, in certain embodiments, such preservation media provided by the invention does not contain egg yolk and optionally may lack (or include) other components noted, supra.

A related aspect of the invention are compositions comprising the preservation media provided by the invention, together with additional components, including, in some embodiments, live sperm. In some embodiments the sperm are enriched (or isolated) from semen. A variety of means of sperm enrichment or isolation are possible, including by centrifugation (such as density gradient centrifugation), swim up, filtration, microfluidics, or a combination thereof. In certain embodiments, the live sperm is mammalian sperm, such as bovine, ovine, equine, porcine, leporine, feline, canine, or primate sperm. In certain embodiments, the sperm is from a human. In some embodiments, the human subject has a reduced sperm count, e.g., is oligospermic, e.g., having a sperm count below about: 20, 19, 18, 18, 16, 15, 14, 13, 12, 11, 10, 9, 8, 7, 6, 5, 4, 3, 2 million sperm per milliliter, e.g., less than 15 million sperm per milliliter. In some embodiments, the sperm is from two or more ejaculates, such as 2, 3, 4, 5, 6, or more ejaculates.

Accordingly, consonant with the preservation media provided by the invention, in certain illustrative embodiments, the invention provides a composition comprising liquid sperm preservation medium together with additional components, including, in some embodiments, live sperm; the liquid sperm preservation medium having a pH of between about: 6.7 and 6.9, consisting essentially of a zwitterionic buffer, glucose at a concentration of between about: 0.25-0.35 M, osmolality between about: 340-360 mOsm/kg, an antibiotic, BSA or HSA at a concentration of about: 2-4% (W/V), wherein when stored up to 12 days at 4° C., at least 60% of sperm are motile upon transfer to capacitation medium, relative to suitable control sperm. While illustrated here in a particular embodiment, components of a composition comprising liquid sperm preservation medium together with additional components, including, in some embodiments, live sperm can be varied as illustrated for preservation media provided by the invention. Concordantly, in certain embodiments, a composition comprising a sperm preservation medium does not contain egg yolk and optionally may lack (or include) other components noted for the preservation media, supra.

Given the preservation media and compositions comprising the preservation media provided herein, the invention also provides related aspects of methods of preserving sperm. Such methods entail contacting sperm, including concentrated or isolated sperm, with a preservation medium provided by the invention. As noted above a variety of sperm can be used, including human sperm, such as sperm from an oligospermic human male. The sperm can be pooled from multiple ejaculates. In certain embodiments, the methods of preserving sperm, entail storing a composition comprising preservation media and sperm, e.g., at about 4° C. for a period of time, such as 4, 5, 6, 7, 8, 9, 10, 11, 12 days, or more, while, in certain embodiment, preserving at least 40, 45, 50, 55, 60, 65, 70, 75, 80%, or more, of motile sperm upon capacitation, relative to suitable control.

In some embodiments, the sperm can be isolated from the preservation media provided by the invention before contacting the sperm with an egg, for example contacting in vitro or in vivo by introduction of the sperm to the reproductive system of the female recipient. In some embodiments, following isolation of the sperm from the preservation media, the sperm can be placed in a capacitation medium.

### Methods of Obtaining Sperm Sample

Various methods of collection of viable sperm are known. Such methods include, for example, masturbation into sterile containers, the gloved-hand method, use of an artificial vagina, and electro-ejaculation. Animal semen can be collected by using artificial vagina, electro-ejaculator, or by massaging the ampule of the animal by hand. It can also be directly collected from any section of the male reproductive tract including testicular sperm, and sperm obtained from caput, corpus or cauda epididymis using different methodologies such as puncture of the testis or epididymis using surgical procedures or removing the testis or epididymis and collecting the sperm in surrounding media. The sperm are preferably collected or quickly transferred into an insulated container to avoid a rapid temperature change from physiological temperatures (typically about 35° C. to about 39° C.). The ejaculate typically contains about 0.5 to 15 billion sperm per milliliter, depending upon the species and particular animal. The number may be reduced if obtained from a subfertile male or male suffering from sperm dysfunction.

The sperm may be freshly collected sample from a source animal (e.g., a mammal), or can be previously thawed or cryopreserved sample. At the time of collection, or subsequently, the collected sperm may be combined with any of a number of various buffers that are compatible with sperm, such as TCA, HEPES, PBS, or any of the other buffers disclosed in U.S. Patent Application Publication No. US 2005/0003472, the content of which is hereby incorporated herein by reference. For example, a bovine semen sample typically containing about 0.5 to about 10 billion sperm cells per milliliter may be collected directly from the source mammal into a vessel containing a buffer to form a sperm suspension. The sperm suspension may also contain a range of other additives to maintain sperm viability. Exemplary additives include protein sources, antibiotics, growth factors, and compositions that regulate oxidation/reduction reactions intracellularly and/or extracellularly. Examples of each of these additives are well known in the art, as demonstrated in the disclosure of, for example, U.S. Application Ser. Nos. US20070092860A1 and US20050244805A1, the content of each of which is hereby incorporated herein by reference. Alternatively, the semen sample may be collected into an empty container and then subsequently contacted with a buffer within several minutes to hours after collection to form the sperm suspension. In some embodiments, the sperm cells can be collected directly into a container containing energy depletion medium (e.g., HTF medium devoid of glucose, pyruvate and lactate) for incubation under energy depletion. In some embodiments, the sperm cells can be collected in an empty container and subsequently incubated under energy depleting conditions.

In some embodiments, sperm collection comprises washing sperm cells prior to carrying out the methods disclosed herein. Generally, washing involves centrifuging a sample of semen or thawed sperm through a diluting wash media, which allows collection of a sperm-rich pellet. After a sperm wash process, or in place of it, a specific procedure for the isolation of the motile sperm from a sample can be done.

In some embodiments, the sperms are isolated from semen prior to use in methods disclosed herein. In some embodiments, sperm with increased function can be further enriched, (for example, enriching sperm with increased motility), from sperm prepared according to methods disclosed herein. Generally, sperm are isolated or enriched by allowing the motile sperm to swim away from the dead sperm, non motile sperm and debris (sperm swim-up), by centrifuging the sperm through a density gradient, or by passing the sperm through a column that binds the dead sperm and debris. Isolating (or enriching) the spermatozoa from semen is performed by a method selected from the wash and spin method, the sedimentation method, the direct swim-up method, the pellet and swim-up method, and the buoyant density gradient method. These methods are well known in the art. They are traditionally used in assisted reproduction techniques and described in detail in “A textbook of In vitro Fertilization and Assisted Reproduction, The Bourn Hall guide to clinical and laboratory practice, editor: Peter R. Brinsden, The Parthenon Publishing Group” (1999). In some embodiments, the sperm prepared by the methods disclosed herein can be further enriched for motile sperms by isolation procedures such as the sedimentation method, the direct swim-up method, the pellet and swim-up method, and the buoyant density gradient method.

The direct swim-up method implies self-selection of motile sperms, essentially comprising layering an aliquot of medium on top of a semen sample or a preparation of sperm disclosed herein and allowing it to stand at room temperature for a certain period of time. The motile sperm cells will migrate into the top layer (medium), from which they can be recovered. The method may also include centrifugation step(s). The advantage of “swim-up” selected spermatozoa is that the motile cells present in the sample are isolated and concentrated and that the proportion of morphologically normal sperm is increased.

The method may be varied and combined with further isolation/separation techniques, depending on the amount of motile cells in the sample. For example, the swim-up procedure may be performed through the layering of 1 ml of medium containing albumin on 1 ml of underlying seminal liquid in a test tube. After one hour of incubation at 37° C. in the air or in 5% CO2 the upper phase of the medium to which the spermatozoa with better motility characteristics have migrated is collected. This technique may also comprise or be combined with a centrifugation step, for example centrifugation on Percoll gradients. In typical applications, a sperm containing solution is layered over a gradient material, preferably Percoll at 30-90% mixed with 0.05% pectin, and then subjected to centrifugation to collect sperm enriched for improved function. The separated, isolated or enriched spermatozoa are then used in methods disclosed herein or may be cryopreserved before being further processed, for example. In case of the preparation of sperms prepared by methods herein, they can be used for IVF, ICSI or artificial insemination following enrichment steps or may be cryo-preserved for later use, for example. Accordingly, for any of these isolation, or enrichment methods, the sample may be semen, partially purified sperm, purified sperm, or sperm with increased function prepared by methods herein. In some embodiments, the percentage of motile cells is increased by at least 10%, at least 20%, at least 50%, at least 75%, at least 80%, or about 100% after isolating or enriching the sperm using isolation methods, such as direct swim up, the pellet and swim-up method, and the buoyant density gradient method compared to untreated semen sample or unenriched sperm preparation.

In some embodiments, after isolation, enrichment and washing, the sperm pellet can be resuspended in a medium suitable for further processing, including preservation medium, HTF medium for culturing, medium for energy depleting conditions (e.g., HTF devoid of glucose, lactate and pyruvate). As it relates to sperm with increased function prepared by methods disclosed herein, the sperm preparation can be resuspended in preservation medium, HTF medium for culturing, medium for insemination, assays of fertilization potential as described herein, in vitro fertilization, freezing, intrauterine insemination, cervical cap insemination, and the like. The sperm may be added to medium or the medium can be added to the sperm. The medium can be balanced salt solution which may contain zwitterionic buffers, such as TES, HEPES, PIPES, or other buffers, such as sodium bicarbonate. In general, the medium for diluting sperm or culturing sperm, oocytes, embryos or embryonic stem cells is a balanced salt solution, such as M199, Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BWW Medium, and emCare Media (PETS, Canton, Tex.). In some embodiments, TALP or HTF is used for sperm culture medium, and CZB is used for embryo culture medium. The sperm, or embryo of the present disclosure can be preserved in a cryogenic medium comprising a cryoprotectant.

In some embodiments, the sperm is provided or collected in a preservation medium prior to incubating in energy depletion conditions. In some embodiments, the preservation medium is a buffered solution comprising a slightly acidic pH and having an osmolality of between about: 300 and 400 mOsm/kg, e.g., between about: 300-380, 320-370, 330-370, 340-360, e.g., about: 320, 330, 340, 350, 360, 370, or 380, e.g., about 350 mOsm/kg. In some embodiments, the osmolality of the preservation media provided by the invention is between about: 320-340 mOsm/kg. A “slightly acidic” pH means less than 7, but more than 5. In some embodiments, a slightly acidic pH is between about: 6 and 7, e.g., greater than, or about: 6.1, 6.2, 6.3, 6.4, 6.5, 6.7, 6.8, 6.9 and less than 7.0 (such as 6.99), e.g., between about: 6.5 and 6.99, such as between about: 6.7-6.9, e.g., about 6.8. In some embodiments, the preservation medium is a buffered solution having a pH of between about: 6.7 and 6.9, comprising (or consisting essentially of) a zwitterionic buffer, glucose at a concentration of between about: 0.25-0.35 M, an antibiotic, osmolality of between about 340-360 mOsm/kg, BSA or HSA at a concentration of about: 2-4% (W/V), wherein sperm stored in the preservation medium for up to 12 days at 4° C. maintain at least 60% motile sperm upon transfer to capacitation medium, relative to suitable control sperm; optionally wherein the medium does not contain egg yolk. In some embodiments, the sperm is stored in the preservation medium for about 2 hours, about 3 hours, about 4 hours, about 24 hours, about 10 days, about 1 month, about 6 months, about 10 months, about one year or more prior to incubating under energy depleting conditions. In some embodiments, the sperm in preservation medium are washed, centrifuged in a pellet and resuspended in the energy depletion medium. In some embodiments, the sperm with increased function prepared by the methods disclosed herein are further stored in the preservation medium described above.

### Suitable Control Sperm

A suitable control sperm can be sperm incubated under control conditions, i.e., in a control buffer such as, human tubal fluid (“HTF”) medium or modified HTF medium and not in energy depletion conditions. HTF comprises a sodium bicarbonate buffering system and may be utilized for uses requiring a carbon dioxide atmosphere during incubation. Modified HTF comprises a combined sodium bicarbonate and HEPES ([4-2(2-hydroxyethyl)-1-piperazineethanesulfonic acid]) buffer. Suitable examples of HTF medium or modified HTF medium include those that are commercially available from Irvine Scientific, Santa Ana, Calif. In some embodiments, the incubating in energy depletion conditions can be incubating the HTF medium from which glucose, lactate and pyruvate has been omitted. The sperm may be incubated for a period sufficient to provide a measurable change in the motility (or other characteristics) of the sperm; in specific embodiments of the method, incubation is from 1 minute to 24 hours, 15 minutes to 3 hours, 30 minutes to 1.5 hours, about 1 hour, or any subrange or subvalue thereof. In some embodiments, a suitable control sperm is sperm which is incubated in energy depletion conditions followed by treatment with a first energy source (e.g., selected from a gluconeogenesis substrate, or a glycolytic substrate) or a second energy source (e.g., selected from a gluconeogenesis substrate, or a glycolytic substrate but not same as first energy source) independently. In some embodiments, a suitable control sperm is sperm which is incubated in energy depletion conditions followed by treatment with a gluconeogenesis substrate, or a glycolytic substrate independently. In some embodiments, a suitable control is a sperm which is incubated in energy depletion conditions followed by treatment with a first energy source and a second energy source simultaneously. In some embodiments, a suitable control sperm is a sperm which is incubated in energy depletion conditions followed by treatment with a gluconeogenesis substrate and a glycolytic substrate simultaneously. In some embodiments, a suitable control sperm is an untreated sperm. It is understood that a suitable control sperm can be at least one sperm or a population of sperm, for example, a sperm preparation, or a sperm suspension. A suitable control can be sperm incubated under control conditions, i.e., in a control buffer. The control condition can comprise, for example, incubating sperm under standard capacitation conditions. “Standard capacitation conditions” as used herein refers to incubating sperm in standard capacitation media.

### Sperm Preparation

In some embodiments, the invention provides sperm preparations, such as preparations of activated (e.g., sperm having been starved following introduction of an effective amount of both the first and second energy sources, enriched for hyperactivated and intermediate sperm), partially activated sperm (sperm having been starved and contacted with an effective amount of only a first energy source), or potentiated sperm. These are collectively “sperm preparations provided by the invention” or “preparations provided by the invention.” In some embodiments the invention provides preparations of hyperactivated sperm comprising at least 5% hyperactivated sperm, e.g., at least about: 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0%, or more hyperactivated sperm, e.g., between about: 5-20, 8.5-20, 10-20, or 12.5-20%. In some embodiments, the preparation also contains at least about: 5.5, 6.0, 6.5, 7.0, 7.5, 8.0, 8.5, 9.0, 9.5, 10.0, 12, 14, 15, 16, 18, 20, 22, 24, 25, 26, 28, or 30% intermediate motility sperm, e.g., between about 20.5-30%, 22.5-30%. Thus, in some embodiments, the percentage sum of hyperactivated and intermediate motility sperm is at least: 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21.0, 21.5, 22.0, 22.5, 23.0, 23.5, 24.0, 24.5, 25, 26, 27, 28, 29, 30, 35, 40, 41, 42, 43, 44, 45, 46, 47, 48, 49, 50%, or more, e.g., between about: 10-50, 30.5-50, 32.5-50. As the skilled artisan will appreciate, sperm may be separated based on hyperactivation (and/or intermediate) phenotype, but in some embodiments, the foregoing percentages are based on preparations that have not been activated and then sorted based on hyperactivation (however, in some embodiments, sperm preparations may have been pre-processed, e.g., to separate or otherwise enrich sperm from other seminal components, including certain irregular sperm). In some embodiments, the hyperactivated (or intermediate motility, or hyperactivated and intermediate motility) sperm in the preparation have 10, 15, 20, 25, 30, 35, 40, 45, 50%, or more (e.g., 2, 3, 4, 5, 6, 7, 8, 9, 10-fold, or more) reduction in intracellular RNA concentration (such as small non-coding RNAs, including microRNA), relative to a suitable control. In some embodiments sperm in a preparation provided by the invention are characterized (as assessed by either bulk average metrics or percentages in categories) by altered sperm head morphology, increased tail movement (e.g., amplitude), or a combination thereof.

In some embodiments, the disclosure provides a preparation of sperm comprising a different epigenetic profile than a suitable control sperm.

“Epigenetic profile,” is defined as DNA, RNA or protein modifications (e.g., methylation or acetylation) that do not involve an altered nucleotide sequence. Non-limiting examples of modifications include methylation and/or acetylation and/or binding of non-coding RNA and/or histone modifications. Non-coding RNA (e.g., miRNA, piRNA, snoRNA, endo-siRNA) binding encompasses genetic signaling of spliced intronic or exonic RNA and generation of single or double stranded RNA entities including RNAi-like entities. “Epigenetic profile” may also include protein modifications e.g., protein methylation, protein acetylation e.g., include histone modification, such as changes in acetylation, methylation, and the like.

In some embodiments, the sperm treated with the methods disclosed herein exhibit a different or altered epigenetic profile relative to a suitable control (e.g., a suitable control sperm). As used herein, the term “different epigenetic profile” refers to a change in pattern of one or more modifications to the DNA, RNA, and/or protein. In some embodiments, a change in pattern comprises a presence of a modification (e.g., methylation or acetylation) at a specific site on the DNA, RNA or protein of the sperm relative to that of a suitable control sperm. In some embodiments, a change in pattern comprises an absence of a modification (e.g., methylation or acetylation) at a specific site on the DNA, RNA or protein of the sperm relative to that of a suitable control sperm. In some embodiments, a change in pattern comprises an altered level of one or more modifications. In some embodiments, a change in pattern comprises an increase in level of one or more modifications. In some embodiments, a change in pattern comprises a decrease in level of one or more modifications. For example, an altered level of one or more modification can comprise, an increase or decrease in methylation level, acetylation level and the like. In some embodiments, the different epigenetic profile comprises an altered level of DNA methylation and/or DNA acetylation. In some embodiments, the different epigenetic profile comprises a presence and/or absence of methylation at a specific DNA site. In some embodiments, the different epigenetic profile comprises a presence and/or absence of acetylation at a specific DNA site. Methods to measure epigenetic changes are well known in the art. See e.g., Stephens K. E. et al., Biol Res Nurs. 2013 October; 15(4): 373-381, and DeAngelis T. J. Mol Biotechnol. 2008 February; 38(2): 179-183, which are incorporated herein by reference in their entireties.

In some embodiments, the different epigenetic profile in a sperm sample relative to a control sperm is associated with a physiological condition, trait, phenotype or state. For example, the different epigenetic profile can be associated with absence of a condition such as obesity (or obesity-associated disorder such as cancer or diabetes) or presence of a desirable trait such as increased milk production, or absence of a non-desirable trait such as decreased fertility. Accordingly, the sperm treated with the methods disclosed herein can be useful for producing an offspring with improved fitness relative to a parent, either male or female.

In some embodiments, the invention provides a preparation of sperm prepared by any one of the methods provided by the invention.

In some embodiments, the invention provides preparations of sperm prepared by enriching sperm from semen of a male subject, such as a normospermic male, sub fertile male, or oligospermic male, e.g., a subfertile (including oligospermic) male, incubating the sperm under energy depletion for a time suitable to potentiate the sperm and providing the sperm with a first energy source selected from: an effective amount of a glycolytic energy source or an effective amount of a gluconeogenesis substrate, but not an effective amount of both a glycolytic energy source and gluconeogenesis substrate.

For any of the preparations provided by the invention, sperm can be from any male subject, such as a mammal, and in some embodiments, a human. In some embodiments, the human is a normospermic male, or in other embodiments, the male is an oligospermic or subfertile (e.g., low sperm motility) subject.

In some embodiments, the sperm preparations described herein can be preserved with sperm preservation media provided by the invention. In some embodiments, the sperm preparations provided herein can be prepared by various methods provided by the invention (e.g., enhancing sperm function, promoting fertilization, etc.), which methods can, in some embodiments, be performed using the various kits provided by the invention to then, in certain embodiments, produce the sperm preparations provided by the invention, and/or in additional methods provided by the invention, such as methods of fertilization, including methods of assisted reproduction.

### Promoting Fertilization

The preparations of sperm with increased function prepared by the methods disclosed herein can be useful to promote fertilization. Accordingly, the present disclosure also relates to methods of promoting fertilization. The methods comprise incubating a sperm under energy depleting conditions to potentiate the sperm, providing the potentiated sperm with a first energy source and a second energy source in a serial manner to increase one or more sperm function, and providing the sperm with increased function with access to an egg under conditions to promote fertilization. The preparation of sperm with increased function can be applied in IVF, ICSI, artificial insemination (e.g., intra-uterine insemination) in human as well as in the biomedical research industry of animal models for human diseases (infertility, sperm dysfunction), and in the breeding and agricultural industries. The sperm with increased function prepared by the methods disclosed herein, can be provided access to an unfertilized egg of the same species as the sperm to promote in vitro fertilization, ICSI, or can be used for artificial insemination, including for example, intrauterine insemination of female subjects of the same species as the sperm.

### In Vivo Fertilization

The sperm with increased function prepared by the methods disclosed herein can be useful to promote fertilization in vivo by providing the sperm with increased function access to an egg, e.g., in the reproductive tract of a female subject of the same species as the sperm. In vivo fertilization can be done by artificial insemination of sperm, for example, by intracervical insemination or intrauterine insemination. Standard artificial insemination and intrauterine insemination, and other methods are well known to those of skill in the art. In some embodiments, the sperm with increased function is provided access to an egg in the reproductive tract of a female subject by intrauterine insemination of the said sperm to promote fertilization of the egg. In other embodiments, the sperm can be provided the second energy source and access to an egg in vivo by intrauterine insemination of a mammalian sperm which has been incubated under energy depletion conditions and provided the first energy source in vitro. The sperm that is injected, may be used as held in suitable liquids. Liquid used for this purpose may be those liquids generally used as a medium for artificial insemination.

### In Vitro Fertilization

The present methods and preparations of sperm disclosed herein are useful in promoting fertilization by assisted reproductive technology, e.g., embryo viability following ART, and in particular IVF. Other suitable ART techniques to which the present disclosure is applicable include, but are not limited to, gamete intrafallopian transfer (GIFT), zygote intrafallopian transfer (ZIFT), blastocyst transfer (BT), intracytoplasmic sperm injection (ICSI), gamete, embryo and cell cryopreservation, in vitro preparation of embryos for embryo biopsy and other forms of embryo micromanipulation including formation of embryos by nuclear transfer and production transgenic lines and genetically modified lines. It is also applicable to production of embryonic stem cell lines.

In some embodiments, the sperm with increased function prepared by the methods disclosed herein can be used to fertilize an egg in vitro, such as for example, by microinjection, including intracytoplasmic sperm injection (ICSI), and other methods well known to those in the art. Typically, in IVF, after fertilization, the cells are grown to the blastocyst stage and then implanted. The methods disclosed herein result in increase in formation of an embryo with longer viability and increased ability to develop into a 2-cell stage, blastocyst stage. Accordingly, the preparation of sperm disclosed herein can be useful in vitro fertilization procedures, including, for example ICSI.

The methods of the present disclosure encompass providing the sperm prepared by methods herein with access to an egg to promote in vitro fertilization. Providing the sperm access in vitro to the egg may be carried out in an appropriate medium. The medium used for this purpose can be a medium generally used as a medium for in vitro fertilization, for example, HTF medium. Temperature conditions for providing access may be a general temperature to be used in vitro fertilization, for example, can be an average body or a temperature close thereto of the mammal. Time for providing access may be any time that is generally required in vitro fertilization, but not particularly limited, and preferably from 6 to 24 hours. In vitro fertilization rate can be determined by incubating one or more sperms with matured oocytes for about 24 hr. Oocytes are then be stained with a 1% aceto-orcein stain to determine the percent fertilized or left in culture to divide and the number of embryos formed are counted. Oocytes can be matured in vitro in M199 media with 50 μg luteinizing hormone/ml (Brackett and Zuelke, Theriogenology 39:43, 1993).

### Fertilization Uses

These methods and preparation of sperm disclosed herein are generally applicable to many species, including human, bovine, canine, equine, porcine, ovine, avian, rodent and others. Although useful whenever fertilization is desired, the present methods have particular use in animals and humans that have a fertilization dysfunction in order to increase the likelihood of conception. Such dysfunctions include low sperm count, reduced motility of sperm, and abnormal morphology of sperm. Accordingly, the methods disclosed herein can be useful for preparation of sperm with increased function in infertility clinics prior to their use in vitro fertilization or intrauterine insemination. The methods described herein can be used to improve artificial insemination, IVF or ICSI in exotic species and/or endangered species. As such the methods can find use for promoting fertilization in animals maintained captive in a zoo, and in conservation programs aiming to improve reproduction in animals that are close to extinction in the wild. For example, the methods and preparation of sperm of the present disclosure can be used to improve fertilization and pregnancy rates in animal husbandry, for species of agricultural value, and in species bred for conservation purposes.

In addition, the methods and compositions of the present invention are useful in artificial insemination procedures, e.g., in commercial breedings. The method can be carried out with sperm from domesticated animals, especially livestock, as well as with sperm from wild animals (e.g., endangered species). For example, as disclosed herein, embodiments of the methods and compositions of the disclosure find application in bovine reproduction. The methods and preparation can be useful for artificial insemination in the livestock production industry where it is desirable to influence the outcome towards offspring having one or more preferred characteristics or traits by introducing specific genetically-determined traits into the livestock, e.g., offspring of a particular gender, offspring with enhanced milk production, offspring for quality meat production. Use of the methods described herein will result in improved pregnancy rates. Mammalian sperm are frequently damaged by freezing and thawing and results in lower fertility. By improving the performance of the viable sperm, sperm prepared by methods disclosed herein when used for insemination may promote a higher pregnancy rate per estrus cycle, reducing the number of cycles required to ensure conception and hence reducing the overall cost of artificial insemination.

Semen from animals with highly desirable traits could be used to inseminate more females because fewer cycles would be needed to ensure conception in any one female. For such applications, the semen is obtained from a male with desired characteristics. In order to influence gender outcome of the resulting offspring, the sperm preparation can be sorted into X- and Y chromosome bearing cells, and/or enriched for sperm with one or more increased sperm function disclosed herein. The sperm may be sorted by commonly used methods, for example, as described in Johnson et al. (U.S. Pat. No. 5,135,759) using a flow cytometer/cell sorter into X and Y chromosome-bearing sperm enriched populations. The sperm prepared by the methods disclosed herein can be sorted the into a population comprising a certain percent X chromosome bearing or Y chromosome bearing sperm cells. For example, the spermatozoa of one of the populations may comprise at least about 65% X chromosome bearing or Y chromosome bearing sperm cells, at least about 70% X chromosome bearing or Y chromosome bearing sperm cells, at least about 75% X chromosome bearing or Y chromosome bearing sperm cells, at least about 80% X chromosome bearing or Y chromosome bearing sperm cells, at least about 85% X chromosome bearing or Y chromosome bearing sperm cells, at least about 90% X chromosome bearing or Y chromosome bearing sperm cells, or even at least about 95% X chromosome bearing or Y chromosome bearing sperm cells. In some embodiments, the sorting can be done prior to preparing the sperm with increased function as disclosed herein. In some embodiments, the sorting can be done prior to providing the sperm with increased function with access to an egg for fertilization as in IVF, ICSI or AI.

The methods and preparations provided by the invention can be used in assisted fertilization, such as IVF, including by ICSI (intracytoplasmic sperm injection). In some embodiments, any of the methods provided by the invention can include the step of providing the sperm to a female reproductive tract, optionally wherein the effective amount of a second energy source is provided in the female reproductive tract. In some embodiments, a sperm preparation provided by the invention (having increased sperm function) can be provided access to an egg for a time sufficient to fertilize the egg, which egg may be ex vivo (e.g., IVF, including ICSI) or, in some embodiments, in a female reproductive tract. Such methods, in some embodiments, entail a subsequent implantation of the fertilized egg in a female carrier.

In some embodiments, the invention provides methods of fertilization comprising providing a preparation provided by the invention that has not been contacted with an effective amount of a second energy source with access to an egg and an effective amount of a second energy source so as to provide an effective amount of both a gluconeogenesis substrate and a glycolytic energy source for a time sufficient to fertilize the egg. In some embodiments, these methods are performed in vitro. In other embodiments, these methods are performed in vivo, in the reproductive tract (vagina or uterus) of a female.

In some embodiments, the invention provides methods of fertilization comprising providing a preparation of sperm with a different epigenetic profile. In some embodiments, the different epigenetic profile in a sperm sample relative to a control sperm is associated with a physiological condition, trait, phenotype or state. For example, the different epigenetic profile can be associated with absence of a condition such as obesity (or an obesity-associated disorder, such as cancer or diabetes) or presence of a desirable trait such as increased milk production, or absence of a non-desirable trait such as decreased fertility. Accordingly, the sperm treated with the methods disclosed herein can be useful for producing an offspring with improved fitness than a parental male subject. In one aspect provided herein is a method for producing an offspring with improved fitness than a parental male subject comprising treating the sperm sample from the parental male according to the methods disclosed herein and fertilizing an egg with the treated sperm to generate an embryo, and growing the embryo in a female subject to produce the offspring with improved fitness. The term “offspring with improved fitness” refers to an offspring exhibiting desirable change or improvement in a physiological condition, trait, phenotype or state relative to that in a parental subject. For example, a desirable change can include absence of a condition such as obesity (or an obesity-associated disorder such as cancer, cardiovascular disease, infertility and the like). For example, an improvement can include presence of a desirable trait such as increased milk production.

### Articles of Manufacture

In some embodiments, the invention also provides articles of manufacture and kits, e.g., suitable for performing any of the methods provided by the invention or preparing any of the preparations provided by the invention. For example, in some embodiments, the invention provides articles of manufacture comprising a sperm potentiating solution that, upon contact with sperm, induces energy depletion; a first solution providing a first energy source selected from: an effective amount of a glycolytic energy source or an effective amount of a gluconeogenesis substrate, but not an effective amount of both a glycolytic energy source and gluconeogenesis substrate; and a second solution providing an effective amount of a second energy source. In some embodiments, the articles of manufacture further include a means for isolating or enriching sperm, such as, in some embodiments, a sperm isolating matrix. In some embodiments, the sperm isolating matrix is silanized silica, optionally wherein the silanized silica is in media substantially free of any glycolytic energy source or gluconeogenesis substrate. In some embodiments, the kit comprises instructions for carrying out the methods disclosed herein. The kit can also include a washing medium, a preservation medium, culture medium (e.g., HTF), a diluent, and the like. The kits can further contain adjuvants, reagents, and buffers as necessary.

In certain embodiments, all components and reagents of the kits disclosed herein meet at least United States Pharmacopeia (USP) monograph-grade purity for the component. For some components a USP monograph may not be available, and thus, in certain embodiments, a suitable pharmaceutical grade reference standard purity of the component is used. In some embodiments, the purity of components of kits are a purity of about 95%. The components and reagents more particularly have a purity of at least about 95%, more particularly at least about 98%, more particularly at least about 99%. In some embodiments, the components and reagents of the kits are substantially sterile, substantially pyrogen free or substantially sterile and substantially pyrogen free.

In some embodiments, the components included in the reagents of the kits are substantially pure. As used herein, substantially pure means sufficiently homogeneous to appear free of readily detectable impurities as determined by any appropriate method, e.g., column chromatography, gel electrophoresis, or HPLC. The term “substantially pure” means a preparation which is at least 60% by weight (dry weight), the component of interest (e.g., glucose or pyruvate). In particular embodiments the preparation is at least 75%, more particularly at least 90%, and still more particularly at least 99%, by weight the component of interest. Where a preparation includes two or more components of interest a “substantially pure” preparation means a preparation in which the total weight (dry weight) of all components of interest is at least 60% of the total dry weight. Similarly, for such preparations containing two or more components of interest, the total weight of the two or more components of interest is at least 75%, more particularly at least 90%, and still more particularly at least 99%, the total dry weight of the preparation. In some embodiments, the disclosure also provides articles of manufacture and kits, e.g., suitable for performing any of the methods provided by the invention or preparing any of the preparations provided by the invention.

For example, in some embodiments, the disclosure provides a kit comprising a first container comprising a sperm potentiating energy depletion composition that, upon contact with sperm, induces energy depletion and generates a potentiated mammalian sperm.

In some embodiments, the sperm potentiating energy depletion composition comprises a low glucose concentration, e.g., less than about: 0.5, 0.4, 0.3, 0.2, 0.1, 0.09, 0.08, 0.07, 0.06, 0.05, 0.04, 0.03, mM glucose, or less, such as less than about: 0.02 or 0.01 mM, e.g., less than about 0.01 mM. In some embodiments the sperm potentiating energy depletion composition is substantially glucose-free. In some embodiments, the sperm potentiating energy depletion composition comprises a low pyruvate concentration, e.g., less than about: 0.15, 0.10, 0.09, 0.08, 0.07, 0.06, 0.05, 0.04, 0.03, 0.02, 0.01, 0.005, 0.003, 0.002 mM, or less. In some embodiments the sperm potentiating energy depletion composition is substantially pyruvate-free.

In some embodiments, the sperm potentiating energy depletion composition is substantially free of carbon sources, such as low glucose concentration and low pyruvate concentration, e.g., is substantially glucose-free and substantially pyruvate-free. In some embodiments, the sperm potentiating energy depletion composition comprises a buffer. In some embodiments, the buffer is HEPES, MOPS, or a combination thereof. In some embodiments, the sperm potentiating energy depletion composition comprises HEPES in a concentration of about: 1 mM-50 mM, 2-40 mM, 3-30 mM, 5-20 mM, 7-15 mM or 7.5-12.5 mM. In some embodiments, the HEPES is at a concentration of about: 1, 2, 5, 6, 7, 8, 9, 10, 11, 12, 15, 20, 25, 30, 35, 40, 45, or 50 mM. In some embodiments, HEPES is at a concentration of at least about: 1, 2, 5, 6, 7, 8, 9, 10, 11, 12, 15, 20, 25, 30, 35, 40, 45, or 50 mM. In some embodiments, the HEPES is at a concentration of 10 mM.

In some embodiments, the sperm potentiating energy depletion composition further comprises a serum albumin e.g., human serum albumin, fetal bovine serum, or bovine serum albumin. In some embodiments, the sperm potentiating energy depletion composition comprises human serum albumin (HSA), e.g., at a concentration of about: 1-10 mg/ml, 2-8 mg/ml, or 3-7 mg/ml. In some embodiments, the HSA is at a concentration of about: 1, 2, 3, 4, 5, 6, 7, 8, 9, or 10 mg/ml. In some embodiments, the HSA is at a concentration of at least about: 1, 2, 3, 4, 5, 6, 7, 8, 9, or 10 mg/ml. In some embodiments the HSA is at a concentration of 4 mg/ml. In some embodiments, the serum albumin, such as a human serum albumin, is provided in a separate solution, such as a concentrated stock solution, and diluted into one or more of the compositions in a kit provided by the invention

In some embodiments, the sperm potentiating energy depletion composition further comprises an antibiotic. In some embodiments, the antibiotic is present in the sperm potentiating energy depletion composition at a concentration of about: 1-20 μg/ml, 2-18 μg/ml, 4-16 μg/ml, 6-14 μg/ml, or 8-12 μg/ml. In some embodiments, the antibiotic is at a concentration of about: 1, 2, 4, 6, 8, 10, 12, 14, 16, 18, or 20 μg/ml. In some embodiments, the antibiotic is at a concentration of at least about: 1, 2, 4, 6, 8, 10, 12, 14, 16, 18, or 20 μg/ml. In some embodiments the antibiotic is at a concentration of 10 μg/ml. In some embodiments, the antibiotic is gentamicin or penicillin.

In some embodiments, the sperm potentiating energy depletion composition further comprises one or more salts; e.g., NaCl, KCl, CaCl₂, KH₂PO₄, MgSO₄.7H₂O, NaHCO₃.

In some embodiments, NaCl is present at a concentration of about: 50-150 mM, 60-140 mM, 70-130 mM, 80-120, mM, or 90-100 mM. In some embodiments, the NaCl is present at a concentration of about: 50, 60, 70, 80, 90, 95, 96, 97, 97.1, 97.2, 97.3, 97.4, 97.5, 97.6, 97.8, 97.9, 98, 98.5, 99, 99.5, 100, 110, 120, 130, 140, or 150 mM. In some embodiments, the NaCl is present at a concentration of at least about: 50, 60, 70, 80, 90, 95, 96, 97, 97.1, 97.2, 97.3, 97.4, 97.5, 97.6, 97.8, 97.9, 98, 98.5, 99, 99.5, 100, 110, 120, 130, 140, or 150 mM. In some embodiments, NaCl is present at a concentration of 97.8 mM.

In some embodiments, KCl is present at a concentration of about: 1-10 mM, 1.5-9.5 mM, 2-9 mM, 2.5-8.5 mM, 3-8 mM, 3.5-7.5 mM, 4-7, mM, 4.5-6.5 mM, 4.5-6 mM, or 4.5-5 mM. In some embodiments, the KCl is present at a concentration of about: 1, 2, 3, 4, 4.1, 4.2, 4.3, 4.4, 4.5, 4.6, 4.7, 4.8, 4.9, 5, 5.5, 6, 7, 8, 9, or 10 mM. In some embodiments, the KCl is present at a concentration of at least about: 1, 2, 3, 4, 4.1, 4.2, 4.3, 4.4, 4.5, 4.6, 4.7, 4.8, 4.9, 5, 5.5, 6, 7, 8, 9, or 10 mM. In some embodiments, KCl is present at a concentration of 4.7 mM.

In some embodiments, CaCl₂ is present at a concentration of about: 1-5 mM, 1.1-4.5 mM, 1.2-4 mM, 1.3-3.5 mM, 1.4-3 mM, or 1.5-2.5 mM. In some embodiments, the CaCl₂ is present at a concentration of about: 1.0, 1.2, 1.4, 1.6, 1.8, 2, 2.2, 2.4, 2.6, 2.8, 3, 3.5, 4, 4.5, or 5 mM. In some embodiments, the CaCl₂ is present at a concentration of at least about: 1.0, 1.2, 1.4, 1.6, 1.8, 2, 2.2, 2.4, 2.6, 2.8, 3, 3.5, 4, 4.5, or 5. In some embodiments, CaCl₂ is present at a concentration of 2 mM.

In some embodiments, KH₂PO₄ is present at a concentration of about: 0.1-0.6 mM, 0.15-0.55 mM, 0.2-0.5 mM, 0.25-0.45 mM, or 0.3-0.4 mM. In some embodiments, the KH₂PO₄ is present at a concentration of about: 0.1, 0.15, 0.2, 0.22, 0.25, 0.26, 0.3, 0.32, 0.34, 0.35, 0.36, 0.37, 0.38, 0.39, 0.4, 0.45, 0.5, or 0.6 mM. In some embodiments, the KH₂PO₄ is present at a concentration of at least about: 0.1, 0.15, 0.2, 0.22, 0.25, 0.26, 0.3, 0.32, 0.34, 0.35, 0.36, 0.37, 0.38, 0.39, 0.4, 0.45, 0.5, or 0.6 mM. In some embodiments, KH₂PO₄ is present at a concentration of 0.37 mM.

In some embodiments, NaHCO₃ is present at a concentration of about: 10-50 mM, 12-45 mM, or 15-30 mM. In some embodiments, the NaHCO₃ is present at a concentration of about: 10, 12, 14, 16, 18, 20, 22, 24, 26, 30, 35, 40, 45, or 50 mM. In some embodiments, the NaHCO₃ is present at a concentration of at least about: 10, 12, 14, 16, 18, 20, 22, 24, 26, 30, 35, 40, 45, or 50 mM. In some embodiments, NaHCO₃ is present at a concentration of 20 mM.

In some embodiments, MgSO₄.7H₂O is present at a concentration of about: 0.1-0.5 mM, 0.12-0.45 mM, 0.14-0.4 mM, 0.16-0.35, or 0.18-0.3 mM. In some embodiments, the MgSO₄.7H₂O is present at a concentration of about: 0.1, 0.12, 0.14, 0.16, 0.18, 0.2, 0.22, 0.24, 0.26, 0.28, 0.3, 0.35, 0.4, 0.45, or 0.5 mM. In some embodiments, the MgSO₄.7H₂O is present at a concentration of at least about: 0.1, 0.12, 0.14, 0.16, 0.18, 0.2, 0.22, 0.24, 0.26, 0.28, 0.3, 0.35, 0.4, 0.45, or 0.5 mM. In some embodiments, MgSO₄.7H₂O is present at a concentration of 0.2 mM.

In some embodiments, the sperm potentiating energy depletion composition comprises a pH indicator e.g., phenol red, e.g., at a concentration of about: 0.0001-0.001%, 0.0002-0.009%, 0.0003-0.0008%, 0.0004-0.0007%, or 0.0005-0.00065%. In some embodiments, phenol red is present at a concentration of about: 0.0001%, 0.0002%, 0.0003%, 0.0004%, 0.0005%, 0.0006%, 0.0007%, 0.0008%, 0.0009%, or 0.001%. In some embodiments, phenol red is present at a concentration of at least about: 0.0001%, 0.0002%, 0.0003%, 0.0004%, 0.0005%, 0.0006%, 0.0007%, 0.0008%, 0.0009%, or 0.001%. In some embodiments, phenol red is present at a concentration of 0.0006%.

In some embodiments, the sperm potentiating energy depletion composition is a buffered solution comprising a slightly acidic pH and having an osmolality of between about: 200-280 mOsm (mOsm/kg), e.g., between about: 220-260, 225-255, 230-250 mOsm (mOsm/kg), optionally, wherein upon addition of the first or second energy source, the osmolarity (or osmolality) is increased to at least about: 270, 275, 280, 285, 290, or 295 mOsm (mOsm/kg). A “slightly acidic” pH means less than 7, but more than 5. In some embodiments, a slightly acidic pH is between about: 6 and 7, e.g., greater than, or about: 6.1, 6.2, 6.3, 6.4, 6.5, 6.7, 6.8, 6.9 and less than 7.0 (such as 6.99), e.g., between about: 6.5 and 6.99, such as between about: 6.7-6.9, e.g., about 6.8.

In some embodiments, the sperm potentiating energy depletion composition is a nutrient free synthetic human tubal fluid. In some embodiments, the nutrient free synthetic human tubal fluid comprises NaCl e.g., at a concentration of 97.8 mM, KCl, e.g., at a concentration of 4.7 mM, CaCl₂), e.g., at a concentration of 2 mM, KH₂PO₄, e.g., at a concentration of 0.37 mM, MgSO₄.7H₂O, e.g., at a concentration of 0.2 mM, HSA, e.g., at a concentration of 4 mg/ml, gentamycin e.g., at a concentration of 10 μg/ml, HEPES, e.g., at a concentration of 10 mM, and phenol red, e.g., at a concentration of 0.0006%. In some embodiments, the nutrient free synthetic human tubal fluid consists essentially of NaCl e.g., at a concentration of 97.8 mM, KCl, e.g., at a concentration of 4.7 mM, CaCl₂), e.g., at a concentration of 2 mM, KH₂PO₄, e.g., at a concentration of 0.37 mM, MgSO₄.7H₂O, e.g., at a concentration of 0.2 mM, HSA, e.g., at a concentration of 4 mg/ml, gentamycin e.g., at a concentration of 10 μg/ml, HEPES, e.g., at a concentration of 10 mM, and phenol red, e.g., at a concentration of 0.0006%. In some embodiments, the nutrient free synthetic human tubal fluid consists of NaCl e.g., at a concentration of 97.8 mM, KCl, e.g., at a concentration of 4.7 mM, CaCl₂), e.g., at a concentration of 2 mM, KH₂PO₄, e.g., at a concentration of 0.37 mM, MgSO₄.7H₂O, e.g., at a concentration of 0.2 mM, HSA, e.g., at a concentration of 4 mg/ml, gentamycin e.g., at a concentration of 10 μg/ml, HEPES, e.g., at a concentration of 10 mM, and phenol red, e.g., at a concentration of 0.0006%. In some embodiments, the nutrient free synthetic human tubal fluid is substantially free of carbon sources, such as low glucose concentration, low lactate concentration and low pyruvate concentration, e.g., is substantially glucose-free, substantially lactate-free and substantially pyruvate-free.

In some embodiments, the kit further comprises a second container comprising a second composition comprising a first energy source selected from: a glycolytic energy source or a gluconeogenesis substrate, but not both a glycolytic energy source and gluconeogenesis substrate. In some embodiments, the kit further comprises a third container comprising a third composition comprising a second energy source selected from: a glycolytic energy source or a gluconeogenesis substrate and the selected second energy source is not the one selected as the first energy source.

In some embodiments, the glycolytic energy source is glucose. In some embodiments, the gluconeogenesis substrate is pyruvate. In some embodiments, the kit comprises only the first solution comprising the first energy source. In some embodiments, the first energy source is a gluconeogenesis substrate (e.g., pyruvate). In some embodiments, the first energy source is a glycolytic energy source (e.g., glucose). In some embodiments, the kit comprises both the first solution and the second solution, where for example, the first energy source is glucose and the second energy source is pyruvate. In some embodiments, the kit comprises both the first solution and the second solution, where for example, the first energy source is pyruvate and the second energy source is glucose. In some embodiments, the glycolytic energy source is glucose, e.g., at a concentration of about: 100 mM-1M, 200-900 mM, 300-800 mM, 400-600 mM or 500 mM, e.g., at least about: 100, 200, 300, 400, 500, 600, 700, 800, 900 mM, or 1M. In some embodiments, the gluconeogenesis substrate is pyruvate, e.g., at a concentration of about: 10-50 mM, 15-45 mM, 20-40 mM, or 25-35 mM. In some embodiments, the pyruvate is at a concentration of about: 10, 15, 20, 25, 30, 35, 40, 45, or 50 mM e.g., about: 31, 32, 33, 34, 35, 36, 37, 38, 39, or 40 mM. In some embodiments, the pyruvate is at a concentration of at least about: 10, 20, 30, 40, 50, 60, 70, 80, 90, or 100 mM.

In some embodiments, the kit further includes a means of enriching or isolating sperm, such as a microfluidic device, a density gradient solution, or a sperm isolating matrix. In some embodiments, the sperm isolating matrix is silanized silica, optionally wherein the silanized silica is in media substantially free of any glycolytic energy source or gluconeogenesis substrate. In some embodiments, the sperm potentiating energy depletion composition comprises the silanized silica. In some embodiments, the silanized silica is suspended in an appropriate diluent e.g., the nutrient free synthetic human tubal fluid.

In some embodiments, the kit comprises instructions for carrying out the methods disclosed herein. The kit can also include a washing medium, a preservation medium, culture medium (e.g., HTF), a diluent, and the like. The kits can further contain adjuvants, reagents, and buffers as necessary. If necessary, other additives (e.g., amino acids (e.g., glutamic acid) or free radical scavengers) may be present. Moreover, hormones or other proteins may be added. Such hormones and proteins include luteinizing hormone, estrogen, progesterone, follicle stimulating hormone, human chorionic gonadotropin, growth factors, follicular fluid and oviductin, albumin and amino acids. Typically, glycerol is added in 3% to 15%; other suitable concentrations may be readily determined by methods known in the art. Other agents are typically added at a concentration ranging from about: 0.1% to 5%. Skim milk, gelatin, proteins such as casein or oviductin, may also be added.

Other kits consonant with the invention include those, for example that may not include antibiotic (or provides an antibiotic other than gentamicin) and/or that may or may not include phenol red in one or more (1, 2, or all 3) reagents. Kits that substitute components consonant with parameters described by this disclosure as a whole will be readily appreciated to be part of the invention. Substantially similar kits are specifically envisioned, where “substantially similar” kits encompass those where one or more of the components (i.e., 1, 2, 3, 4, 5, 6, 7, 8 or, if applicable, 9 components) vary from the molar concentration described in these particular embodiments by up to about: 1, 2, 3, 4, 5, 6, 7, 8, 9, 10, 11, 12, 13, 14, or 15%. Both liquid solutions (e.g., with purified water, with adjustment of pH, e.g, with HCl and/or NaOH) and lyophilized compositions are encompassed by these particular exemplifications.

The kits can also include a carrier, package, or container that is compartmentalized to receive one or more containers such as vials, tubes, and the like, each of the container(s) comprising one of the separate elements, such as the sperm potentiating composition, second composition comprising the first energy source, and third composition comprising the second energy source to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, bags, containers, bottles, and any packaging material suitable for use in methods disclosed herein. A kit typically includes labels listing contents and/or instructions for use, and package inserts with instructions for use. A set of instructions can also be included.

The kits disclosed herein can be useful for a variety of applications including, but not limited to processing sperm for IVF and IUI. The kits of the present disclosure are useful for practicing the methods disclosed herein. Disclosed herein is a kit comprising a sperm potentiating energy depletion composition, e.g., a nutrient free synthetic HTF. In some embodiments, a kit of the present disclosure comprises a silane coated silica diluted in nutrient free synthetic HTF. Such a kit can be useful, for example, for the process of separating sperm from sperm samples by density gradient or swim up method. The kits of the present disclosure comprise a sperm potentiating energy depletion composition (e.g., comprising a nutrient free synthetic HTF), and a second composition comprising a first energy source (e.g., glucose). One exemplary use of such a kit is for preparing sperm for IUI. Sperm separated by density gradient or swim up method can be washed or diluted using the sperm potentiating energy depletion composition, e.g., a nutrient free synthetic HTF. The sperm incubated with sperm potentiating energy depletion composition for a suitable time can be provided with an effective amount of a first energy source (e.g., a gluconeogenesis substrate or a glycolytic energy source) to prepare the sperm for IUI. In some embodiments, the sperm incubated with sperm potentiating energy depletion composition for a suitable time can be provided with an effective amount of a first energy source, for example, a gluconeogenesis substrate such as pyruvate or salt thereof to prepare the sperm for IUI. In other embodiments, further to the second composition comprising a first energy source, the kits disclosed herein can comprise a third container comprising a third container comprising a third composition comprising a second energy source. In some embodiments, the first energy source is a gluconeogenesis substrate (e.g., pyruvate) and the second energy source is a glycolytic energy source (e.g., glucose). In other embodiments, the first energy source is a glycolytic energy source (e.g., glucose) and the second energy source is a gluconeogenesis substrate (e.g., pyruvate). Such a kit can be useful, for example, for IVF. The sperm incubated with a sperm potentiating energy depletion composition (e.g., comprising a nutrient free synthetic HTF) for a suitable time can be further provided with an effective amount of a first energy source and sequentially or simultaneously provided with an effective amount of a second energy source to prepare the sperm for IVF.

The components of the kits provide for initial incubation of sperm in nutrient free synthetic HTF that does not contain a glycolytic energy source (e.g., glucose) or a gluconeogenesis substrate (e.g., pyruvate), and then later a glycolytic energy source or a gluconeogenesis substrate are added simultaneously or sequentially, resulting in improved sperm function. Gluconeogenesis substrate means a non-carbohydrate carbon sources that is used in the process of gluconeogenesis. The gluconeogenesis substrate acts as substrate for the gluconeogenic pathway, further acting to facilitate gluconeogenesis. Gluconeogenesis substrate suitable for use in the kits of the present disclosure include, but are not limited to, pyruvate, lactate, succinate, citrate, fumarate, malate, aspartate, glycerol, acetyl CoA, isocitrate, alpha-ketoglutarate, succinyl-CoA, oxaloacetate; or a physiologically acceptable derivative, salt, ester, polymer or alpha-keto analogue of the gluconeogenesis substrate. Any gluconeogenic amino acid, or a physiologically acceptable derivative, salt, ester, or polymer, or alpha-keto analogue thereof is also suitable as a gluconeogenesis substrate. Non-limiting examples of gluconeogenic amino acids include alanine, arginine, asparagine, cystine, glutamine, glycine, histidine, hydroxyproline, methionine, proline, serine, threonine and valine. Non-limiting examples of pharmaceutically acceptable salts of pyruvate are lithium pyruvate, sodium pyruvate, potassium pyruvate, magnesium pyruvate, calcium pyruvate, and zinc pyruvate. In some embodiments, the pyruvate is sodium pyruvate. Non-limiting examples of salts of lactate include sodium lactate, potassium lactate, magnesium lactate, calcium lactate, zinc lactate, and manganese lactate. The pyruvate component of the kit can be substituted with any other gluconeogenesis substrate listed above.

Glycolytic energy source includes carbon sources for glycolysis. Non-limiting examples of glycolytic energy source include monosaccharides (such as fructose, glucose, galactose and mannose) and disaccharides (sucrose, lactose, maltose, and trehalose), as well as polysaccharides, galactose, oligosaccharides, polymers thereof. The glucose component of the kit can be substituted with any other glycolytic energy source listed above.

In some embodiments, the kits comprise additional components, for example, other components upstream and downstream of glycolysis such as NADH, NAD+, citrate, AMP, ADP, or a combination thereof are added in combination with at least the first energy source or the second energy source.

It is understood that physiologically acceptable means non-toxic to sperm, oocytes or embryos, and which additionally improves their function and survival during in vitro and in vivo handling and manipulation.

Non-limiting uses of the sperm potentiating energy depletion composition of the kit include, for example, for isolating sperm using swim up method, as a diluent, for example, for diluting silane coated silica for density gradient, and for washing sperm. Sperm samples can be processed by, for example, separation or washing in a sperm potentiating energy depletion composition, and then can be used in a variety of diagnostic or research protocols including infertility testing, and sperm toxicology testing. Examples of infertility tests include tests of sperm motility, percent living sperm, sperm count, membrane function, penetration rate and in vitro fertilization rate. Protocols available in the art may be used which are suitable for a particular sperm cell type and a particular diagnostic or research application. In some embodiments, incubating a sperm with a sperm potentiating energy depletion composition potentiates the sperm. In some embodiments, providing the potentiated sperm with an effective amount of a first energy source increases sperm function, and prepares sperm for IUI. In some embodiments, providing the potentiated sperm with an effective amount of a first energy source and simultaneously or sequentially providing an effective amount of a second energy source increases sperm function, and prepares sperm for IVF.

In some embodiments, compositions and solutions of the kit are provided in prefilled tubes, in a predetermined volume. The product also can be provided in solution in a dispenser for a particular application. In one embodiment, centrifuge tubes are provided. The kits may be stored under refrigeration or room temperature.

In some embodiments, the kits described herein comprise the preservation media provided by the invention. The kits provided by the invention are useful for performing the methods of the invention (e.g., inducing increased sperm function, promoting fertilization, producing offspring with improved fitness etc.), which methods can, in some embodiments, be performed using the various kits provided by the invention to then, in certain embodiments, produce the sperm preparations provided by the invention, and/or in additional methods provided by the invention, such as methods of fertilization, including methods of assisted reproduction. In some embodiments, the kits provided herein are useful for generating sperm preparations of the present disclosure.

### EXAMPLES

The present disclosure will be described in greater detail by way of the following specific examples. The following examples are offered for illustrative purposes, and are not intended to limit the invention in any manner. Those of skill in the art will readily recognize a variety of non-critical parameters that can be changed or modified to yield alternative embodiments according to the invention. All patents, patent applications, and printed publications listed herein are incorporated herein by reference in their entirety.

### Example 1: Materials and Methods

### Media

Media for human sperm capacitation was Human Tubal Fluid (Complete HTF or C-HTF) medium, containing 97.8 mM NaCl, 4.7 mM KCl, 2 mM CaCl2, 0.37 mM KH2PO4, 0.2 mM MgSO4.7H2O, 25.1 mM NaHCO3-, 0.33 mM Na-pyruvate, 2.78 mM glucose, lactate 21.4 mM and 5 mg/mL human serum albumin (HSA), 10 μg/mL gentamicin and phenol red 0.0006% at pH 7.4 equilibrated with 5% CO2. For sperm starvation treatment glucose, lactate and pyruvate were omitted from the HTF media above (F-HTF, test media).

### Semen Samples

Semen samples were obtained from healthy males or males seeking treatment for infertility by masturbation into sterile containers. Ejaculates were liquified for up to 2 hours prior to processing for the experiment. Following liquefaction, the volume of the ejaculate was divided equally for processing into F-HTF (test) conditions or C-HTF (control conditions). Semen samples were processed by either density-gradient centrifugation or direct swim up method to collect viable sperm cells.

### Sperm Processing

### Density Gradient Centrifugation

Following liquefaction, the entire volume of each ejaculate was equally divided over two different gradient conditions. The test sample was prepared using a 45-90% Percoll (Sigma, P-1644)) gradient in phosphate buffered saline. The control sample was prepared using an Isolate gradient (Irvine Scientific, Santa Ana, Calif.; 99264) in human tubal fluid. Both samples were centrifuged for 20 min at 500×g. Following centrifugation, the supernatant was removed, and the pellet washed with 10 ml media. The test sample was washed in F-HTF and the control sample was washed in C-HTF.

### Sperm Swim Up

Following liquefaction, the entire volume of each ejaculate was divided into a test sample and control sample, as previously described. The test sample was layered gently with 2.5 ml of F-HTF. The control sample was layered with C-HTF medium. Tubes were carefully inclined at a 45° angle and incubated for 1 h at 37° C., 5% CO₂. The supernatant was carefully collected, and washed F-HTF and the control sample was washed in C-HTF.

### Analysis of Sperm Motility

Sperm suspensions of test and control sperm (6 μl) were loaded into one pre-warmed chamber slide (depth, 20 μm) (Leja slide, Spectrum Technologies) and placed on a microscope stage at 37° C. Sperm motility was examined using the CEROS II computer-assisted semen analysis (CASA) system (Hamilton Thorne Research, Beverly, Mass.). One-second tracks were captured using the following settings: 60 frames per second, 60 frames acquired, minimum contrast=80, minimum size=3 pixels, default cell size=6 pixels, default cell intensity=160, slow cells counted as motile, low VAP cutoff=10 μm/s, low VSL cutoff=0 μm/s, minimum intensity gate=0.18, maximum intensity gate=1.21, minimum size gate=0.56 pixels, maximum size gate=2.63 pixels, minimum elongation gate=0 pixels, and maximum elongation gate=99 pixels. Raw data were sorted and analyzed using the CASAnova parameters (Goodson et al., 2018, supra). At least 20 microscopy fields corresponding to a minimum of 500 sperm were analyzed in each experiment.

### Example 2: Experimental Results

This example shows that serial reintroduction of energy source after nutrient depletion increases sperm hyperactivation.

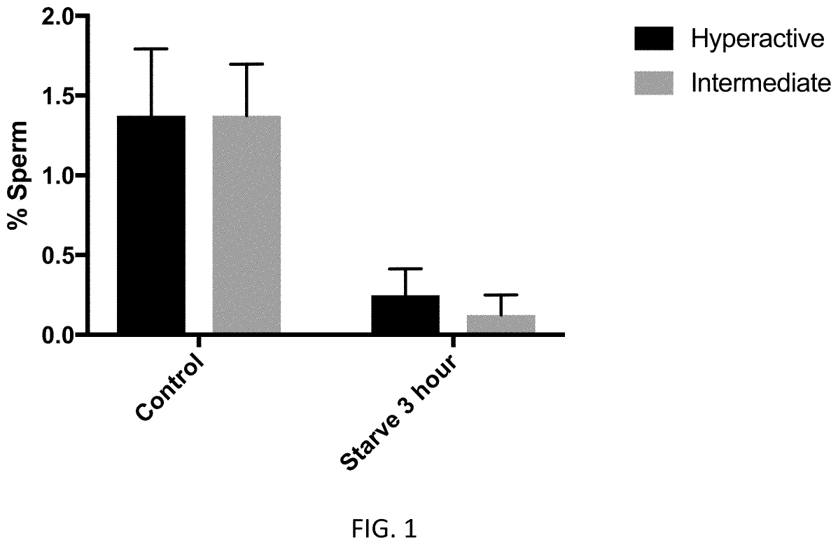

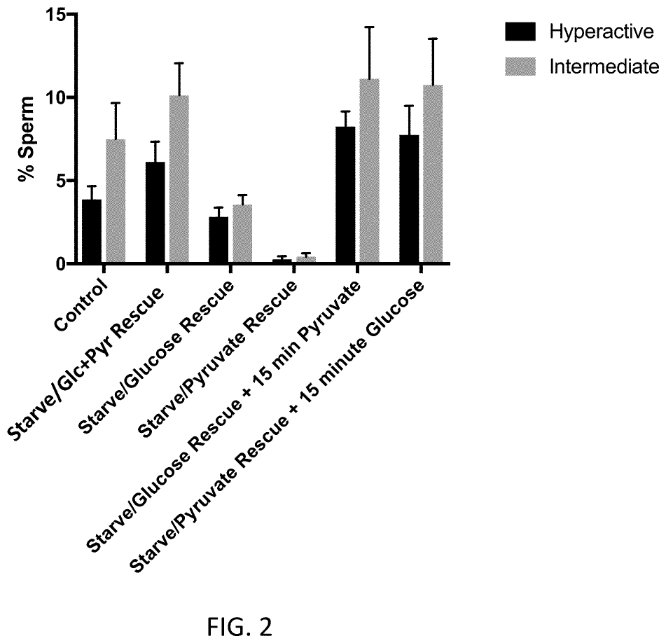

Incubating sperm in a glucose, pyruvate and lactate-free media for three hours resulted in a reduction in motility as shown in FIG. 1. Rescue of sperm motility was tested with different energy substrates. When sperm were starved for 3 hour and rescued with a complete HTF sperm hyperactivation and intermediate motility were elevated compared with the control treatment (FIG. 2). In contrast, sperm treated with glucose (5 mM) or pyruvate (0.33 mM) alone did not improve sperm hyperactivation compared to the control (FIG. 2). Reintroduction of pyruvate alone had no impact on sperm motility from the starvation state, however, reintroduction of glucose alone restored motility to the levels of control (FIG. 2) suggesting that glucose is the major energy source required for sperm hyperactivation. Surprisingly, when pyruvate was added to the glucose-treated sperm or glucose to the pyruvate-treated sperm, this triggered a significant elevation in hyperactivation motility relative to control conditions or when both pyruvate and glucose were reintroduced to sperm at the same time

### Example 3: Enhancing Activation

Osmolarity of C-HTF is approximately 290 mOsm, where F-HTF is approximately 243 mOsm. To illustrate that hypotonic conditions stress sperm such that when reversed, triggers elevated sperm motility and function, sperm are incubated in different conditions that are hypotonic, isotonic, or hypertonic in the presence of a carbon source that is not metabolized efficiently by the sperm such as trehalose, dextran, or other long chain sugar, and impacts on motility observed by CASA analysis during incubation in hypotonic conditions and following return to isotonic conditions. This includes adjusting concentrations of various ions such as calcium, sodium, and potassium during the potentiation phase, and evaluating motility following return to C-HTF. Additionally, impacts of increasing or decreasing the concentration of ions such as calcium, sodium and potassium during both the potentiation phase and the rescue phase are tested, as are the staged addition ions to mimic the ion cycling that occurs in the female reproductive tract during natural conception. In addition to motility, calcium ion flux is assessed. These manipulations, either alone or in conjunction with the described manipulation of glucose and pyruvate enhance the percentage of sperm that achieve hyperactive or intermediate motility.

Although human sperm exhibit reduced motility during the starvation phase of these treatments, the sperm do not completely stop moving suggesting that the cells are utilizing an internal energy source such as glycogen or degrading cellular components such as lipids, proteins, or RNA. Sperm exposed to the starvation phase are assessed for total lipid content, RNA content, and protein content. Proteomic, metabolomic, and lipidomic analysis are performed following the starvation phase, following addition of first energy source, and following addition of second energy source to illustrate intracellular changes associated with sperm motility states. Total RNA (including certain subfractions, such as mRNA or small non-coding RNA, such as microRNA) is measured in sperm treated with control conditions and sperm treated with test conditions, as illustrated in Example 2. The results of this analysis will indicate RNA is being used as an energy source by sperm.

Nutrient depletion and reintroduction can also alter methylation and acetylation patterns of DNA, RNA, and proteins in the sperm in a manner that improves sperm fitness and/or offspring health and fitness. DNA methylation analysis can be performed by bisulfite sequencing DNA from sperm, either bulk or single cell. Changes in sperm DNA methylation will be assessed after nutrient deprivation and after each nutrient reintroduction.

Staging introduction of upstream carbon sources for glycolysis (such as glucose, mannose, fructose, dextrose, or sucrose) and downstream metabolites (such as pyruvate, lactate, succinate, citrate, fumarate, malate) change the rate of conversion of AMP to ATP resulting in improved sperm motility and function as compared to simultaneous addition. ATP and AMP levels are measured in sperm following starvation, introduction of first energy source and introduction of second energy source. Staged introduction of nutrients following starvation increases conversion of AMP to ATP. Example 4

This example provides additional evidence that staged reintroduction of energy sources activates sperm.

Sperm samples from men seeking treatment for infertility were obtained from a fertility clinic. These samples included normally fertile and subfertile sperm. To improve sperm quality, samples were prepared by density gradient centrifugation as described in Example 1. Following liquefaction, the entire volume of each ejaculate was equally divided and subjected to two different density gradient conditions. The test sample was prepared using a 45-90% Percoll (Sigma, P-1644) gradient diluted in phosphate buffered saline solution devoid of nutrients with a final pH of 7.4 (F-PERCOLL). The control sample was prepared using a 45-90% Percoll gradient diluted in phosphate buffered saline solution with nutrients such as (lactate, glucose and pyruvate) with a final pH of 7.4 (C-PERCOLL). Both samples were centrifuged for 20 min at 500×g. Following centrifugation, the supernatant was removed, and the pellet washed with 10 ml media. The test sample was washed in F-HTF and the control sample was washed in C-HTF.

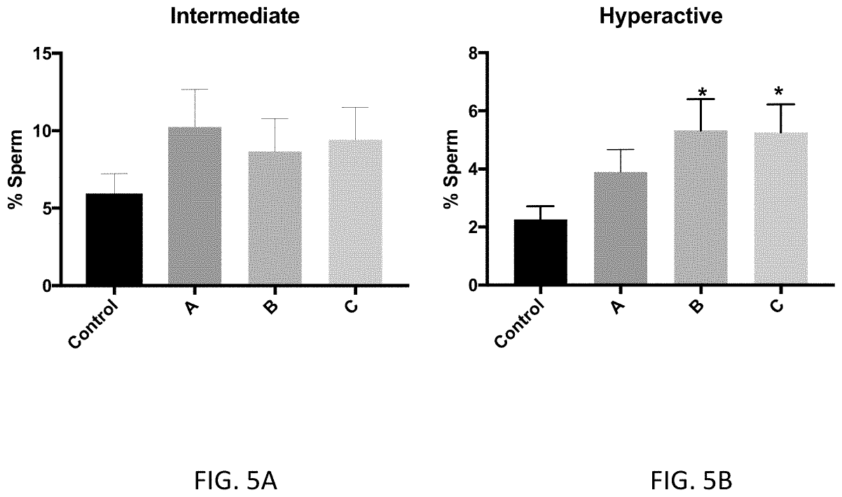

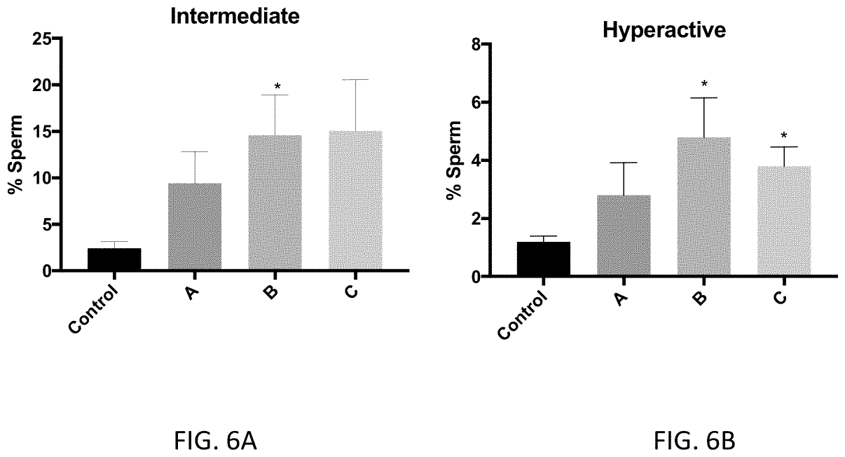

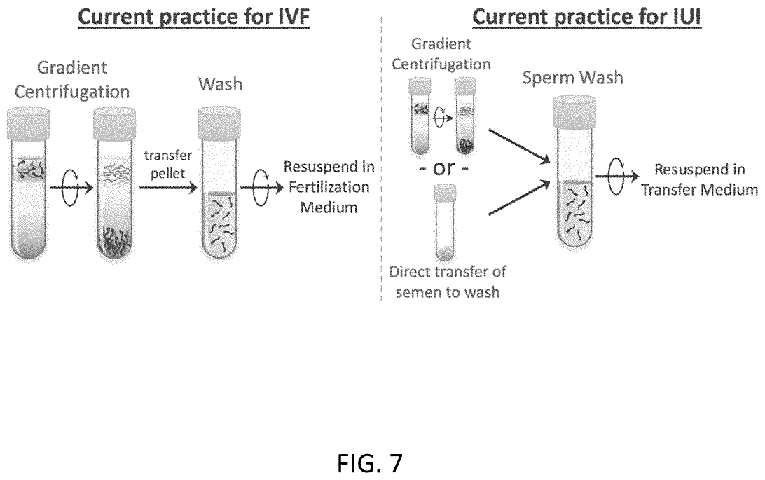

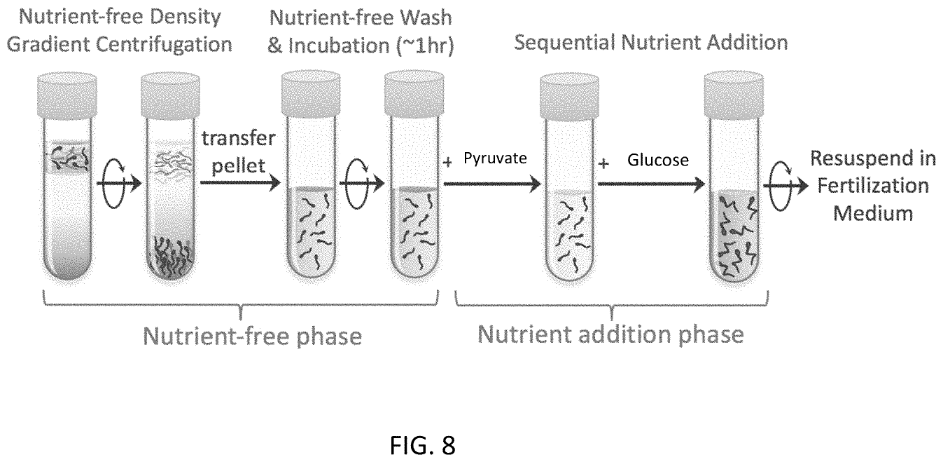

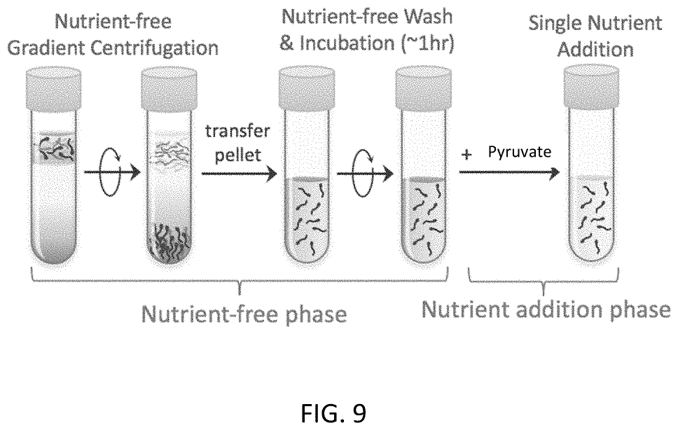

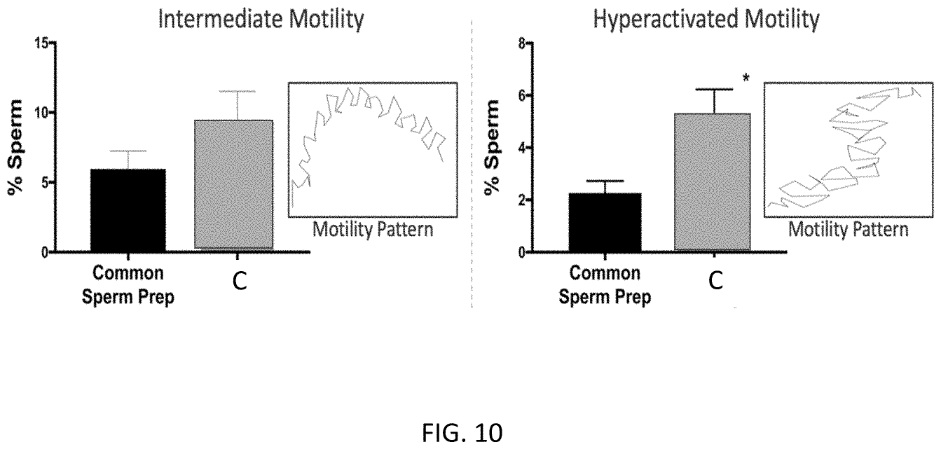

Samples were treated with C-HTF media as described in example 1 or separated by density gradient in a nutrient free media and washed with F-HTF. Sperm with F-HTF A) 1 hour incubation in F-HTF followed by addition of glucose (5 mM), pyruvate (0.33 mM) and lactate incubation for 1 hour 15 minutes, B) 1 hour incubation in F-HTF, addition of pyruvate for 1 hour, then addition of glucose for 15 minutes, or C) 1 hour incubation in F-HTF, addition of glucose for 1 hour then addition of pyruvate for 15 minutes. Samples were analyzed by CASA as outlined in Example 1. Results are shown in FIGS. 5A and 5B, and FIGS. 6A and 6B. Each test condition resulted in an increase in the number of sperm with intermediate and hyperactive motility relative to control, with the highest level of activation observed with treatments B and C.

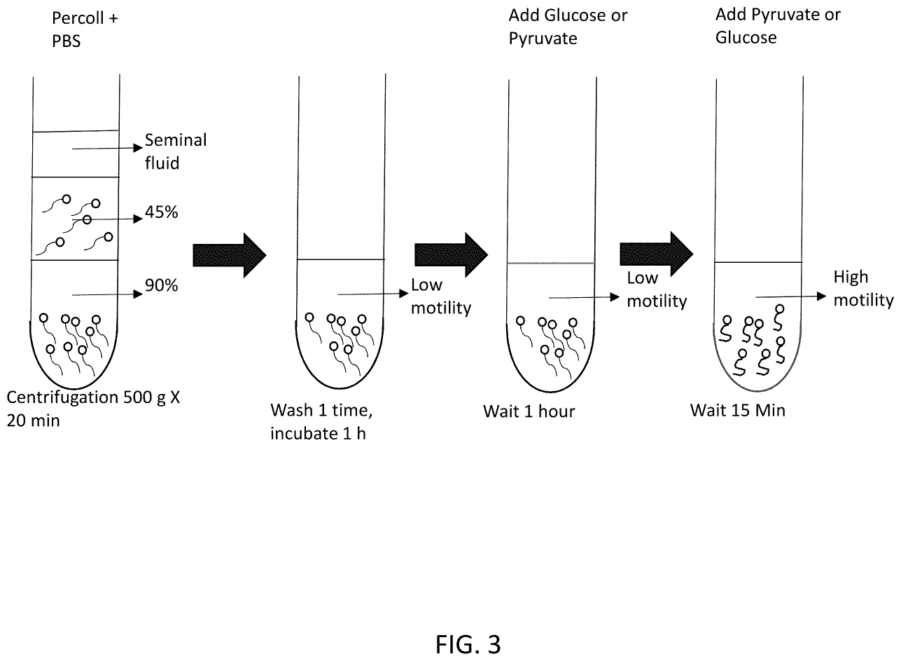

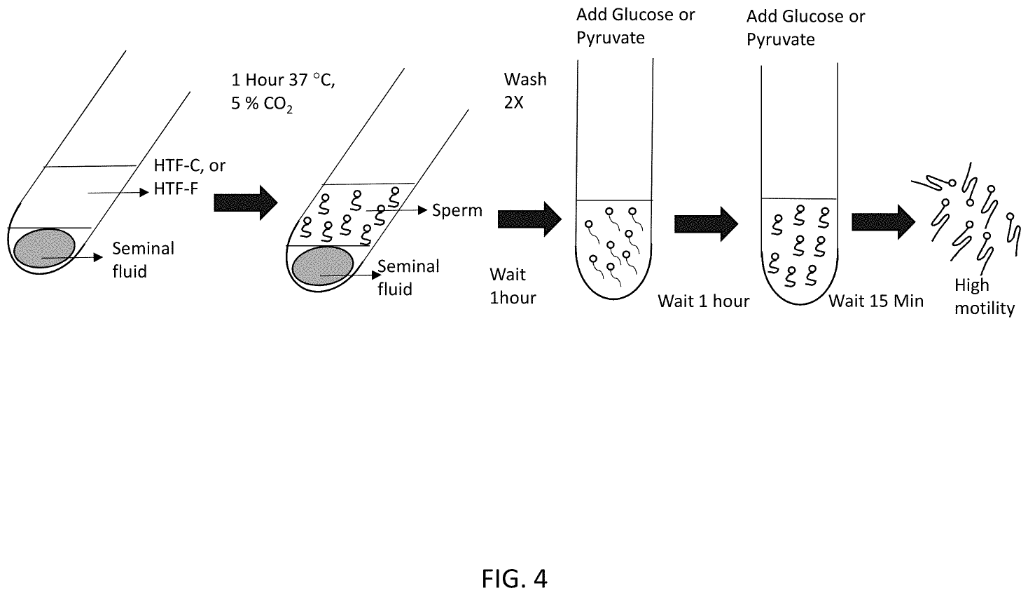

To speed up the starvation state, sperm were separated by density gradient in a nutrient free media and washed with 10 ml F-HTF. After 1-hour incubation in F-HTF, sperm with reduced motility similar to the reduced motility as seen in FIG. 1 were primed with either pyruvate (0.33 mM) or glucose (5 mM) for one hour and then rescued with either (B) glucose (5 mM) or (C) pyruvate (0.33 mM) for 15 minutes as depicted in FIG. 3. Similar to the results shown in FIG. 2, this speed/starve protocol also significantly improved the sperm motility parameters shown in FIG. 5

### Example 5

This example describes use of sperm treated according to certain embodiments of the invention to improve fertility in human subjects undergoing IUI.

Subjects are adult females (e.g., between 18 and 35 years old) without history of recurrent pregnancy loss and may or may not having previously attempted IUI. Subjects are treated with standard of care medicines (e.g., Clomid preparation, with Hcg triggering injection as indicated) and randomly assigned to receive either IUI of sperm prepared by diluting and centrifuging semen on C-HTF or F-HTF and collecting and resuspending cells in C-HTF or F-HTF. Alternatively, the sperm can be collected by density gradient centrifugation and washing and resuspending cells in C-HTF or F-HTF. Sperm are treated with F-HTF (e.g., for 1 hour), then either pyruvate or glucose is added and the sperm are incubated (e.g., for 1 hour), and then the sperm are used for inseminating the female. Sperm are treated with C-HTF (e.g., 2 hours), and then the sperm are used for inseminating the female. Pregnancies are monitored with regular follow-up. Females receiving sperm incubated in the absence of glucose (e.g., 5 mM) or pyruvate (e.g., 0.33 mM) followed by the staged addition of glucose or pyruvate are expected to exhibit a parameter of improved fertility, for example, increased rate of pregnancy, fetal heart rate (e.g., at 7 weeks), ongoing pregnancy (e.g., at 10 weeks) and/or livebirth rates.

### Example 6

This example describes use of sperm treated according to certain embodiments of the invention to improve fertility in human subjects undergoing IVF.

Subjects are adult females (e.g., between 18 and 37 years old) without history of recurrent pregnancy loss and may or may not having previously attempted IVF. Subjects are treated with standard of care medicines (e.g., ovulation suppression followed by ovulation stimulation, with human chorionic gonadotropin triggering injection as indicated) prior to egg retrieval. At egg retrieval, subjects' eggs are randomly assigned to insemination with sperm processed using control conditions or treatment conditions. In the control group, sperm are collected by density gradient centrifugation are resuspended in either sperm wash media, C-HTF or Fertilization media. Non-limiting examples of commercially available fertilization media include Global Total for fertilization (Origio), Continuous Single Culture®-NX Complete (Irvine), Sydney IVF Fertilization Medium (Cook Medical), Irvine Scientific Sperm Wash (Irvine). In the treatment group, sperm are collected by density gradient centrifugation, are washed and resuspended in F-HTF for a sufficient incubation period to potentiate the sperm (e.g., 1 hour). Following this incubation, either pyruvate (0.33 mM) or glucose (5 mM) is added and the sperm are incubated (e.g., for 1 hour). Following this incubation, either glucose (5 mM) or pyruvate (0.33 mM) (whichever was not added in the first step) is added and the sperm are incubated (e.g., at least 15 minutes). For both the treatment and control groups, sperm will be incubated with eggs in vitro and fertilization rates and embryo development monitored. Embryos (e.g., at Day 5) will be transferred to the female and pregnancy will be determined by blood test (e.g., 2 weeks later). Pregnancies are monitored with regular follow-up. Embryos generated with sperm incubated in the absence of glucose and pyruvate followed by the staged addition of glucose and pyruvate are expected to exhibit an improved parameter of fertility, e.g., increased rates of fertilization, blastocyst development. Females receiving embryos generated with sperm incubated in the absence of glucose and pyruvate followed by the staged addition of glucose and pyruvate are expected to exhibit improved pregnancy rate, fetal heart rate (e.g., at 7 weeks), ongoing pregnancy (e.g., at 10 weeks) and/or livebirth rates.

### Example 7 Kits

The kits disclosed herein prepare sperm for IUI or IVF by sequencing of two nutrients in nutrient free sHTF resulting in an increase in the proportion of sperm that exhibit intermediate and hyperactive motility. The components of the kits provide for initial incubation of sperm in nutrient free synthetic HTF that does not contain glucose or pyruvate, and then later glucose and pyruvate are added sequentially. The starve-refeed method generates greater proportions of intermediate and hyperactivated sperm compared to standard sperm preparation (FIG. 10).

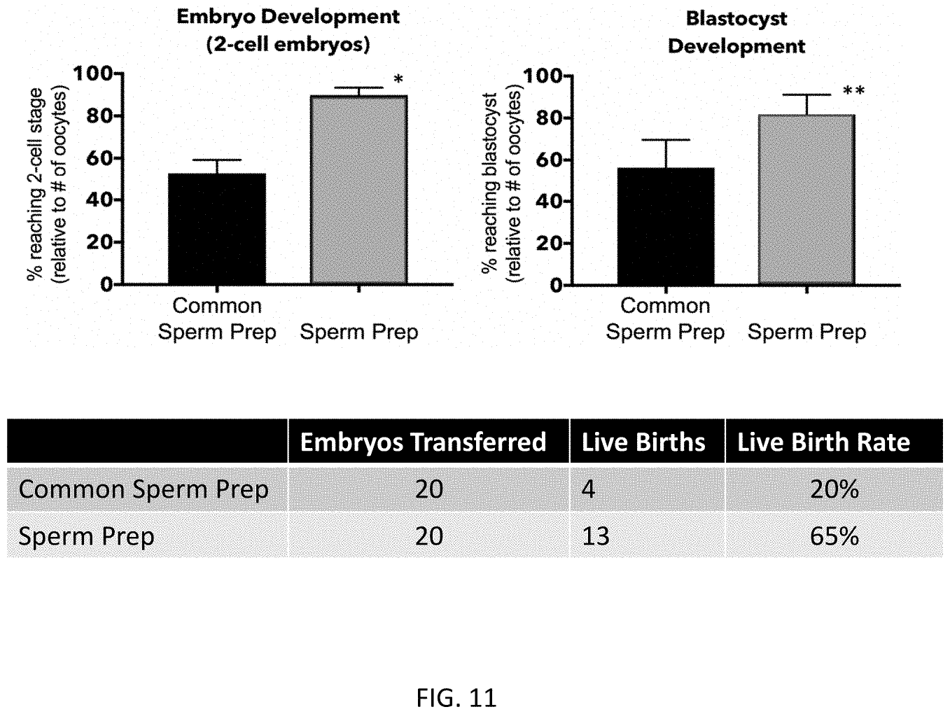

Sperm isolated from the epididymis from a sub-fertile strain of mouse were incubated in nutrient free synthetic HTF that does not contain glucose or pyruvate, and then later provided with glucose and pyruvate sequentially to result in increased proportion of sperm that exhibit hyperactive motility, and subsequently increased the fertilization rate, development to blastocyst, and live birth rate (FIG. 11). Abnormal motility phenotypes were not observed as a result of this nutrient sequencing in mice sperm or human sperm, and abnormalities in embryo development or pups in mice were also not been observed. Based on results and the known association of sperm motility with fertilization, the use of kits disclosed herein can increase the probability of pregnancy and live births for couples undergoing IVF and IUI.

The kits described here are useful for processing and preparing sperm for IVF and IUI. The kits can be maintained as separate kits or can be combined. For example, a kit for density gradient separation can be separate from a kit for IVF or a kit for IUI, or a kit for density gradient separation can be combined with a kit for IVF or IUI. In such combined kit embodiments, a kit for IVF would comprise components from the separate kits, i.e., components for density gradient separation and components for IVF. A kit for density gradient is used in the process of separating sperm from the ejaculate by the density gradient method. A kit for IVF consists of nutrient free sHTF. This reagent is useful for washing sperm to prepare sperm for the fertilization step in IVF and can be used in sperm separation to either (1) isolate motile viable sperm by swim-up or (2) dilute the reagent of a kit for density gradient separation. A kit for density gradient separation includes silane-coated silica in nutrient free sHTF.

A kit for IUI includes nutrient free sHTF. This component can be used for washing sperm, holding sperm for IUI procedure, and, like IVF, can be used in sperm separation to either (1) isolate motile viable sperm by swim-up or (2) dilute density gradient components of the kits. A density gradient reagent of the kit can be silane-coated silica in nutrient free sHTF. Exemplary compositions of the kits are provided in Tables 1-5 below. The tables provide exemplary components of individual reagents and their concentrations

Table 1 below lists the general reagents for certain exemplary kits

| Kit name | Kit for density gradient | Kit for IVF | Kit for IUI |
|---|---|---|---|
| Reagent 1 | Silane-coated | Nutrient-free | Nutrient-free |
| | silica in | sHTF | sHTF |
| | Nutrient-free sHTF | | |
| Reagent 2 | | Pyruvate | Pyruvate |
| Reagent 3 | | Glucose | |

Table 2 lists an exemplary composition of reagents included in kit for IVF and kit for IUI.

| | | Kit for | | |
|---|---|---|---|---|
| | | Density | Kit for | Kit for |
| Reagent | Composition | Gradient | IVF | IUI |
| Sperm | Silanized silica gel | X | | |
| isolation | suspension in nutrient | | | |
| reagent | free sHTF | | | |
| Nutrient-free | 97.8 mM NaCl | | X | X |
| sHTF | 4.7 mM KCl | | | |
| (Reagent 1) | 2 mM CaCl₂ | | | |
| | 0.37 mM KH₂PO₄ | | | |
| | 0.2 mM MgSO₄•7H₂O | | | |
| | 20 mM NaHCO₃ | | | |
| | 4 mg/mL human | | | |
| | serum albumin (HSA) | | | |
| | 10 μg/mL gentamicin | | | |
| | 0.0006% phenol red | | | |
| | 10 mM HEPES | | | |
| Glucose | 500 mM in water | | X | |
| (Reagent 3) | | | | |
| Pyruvate | 33 mM in water | | X | X |
| (Reagent 2) | | | | |

Table 3 lists an exemplary composition of reagent 1 (i.e., nutrient free sHTF)

| Component | Concentration (g/L) |
|---|---|
| NaCl | 5.17 |
| KCl | 0.35 |
| KH₂PO₄ | 0.0502 |
| MgSO₄•7H₂O | 0.0492 |
| CaCl₂•2H₂O | 0.294 |
| NaHCO₃ | 1.68 |
| HEPES | 0.953 |
| Gentamicin Sulfate | 0.010 |
| Phenol Red | 0.010 |

Table 4 lists an exemplary composition of reagent 2

| Component | Concentration (g/L) |
|---|---|
| NaCl | 5.17 |
| KCl | 0.35 |
| KH₂PO₄ | 0.0502 |
| MgSO₄•7H₂O | 0.0492 |
| CaCl₂•2H₂O | 0.294 |
| NaHCO₃ | 1.68 |
| HEPES | 0.953 |
| Sodium pyruvate | 0.145 |
| Gentamicin Sulfate | 0.010 |
| Phenol Red | 0.010 |

Table 5 lists an exemplary composition of reagent 3)

| Component | Concentration (g/L) |
|---|---|
| NaCl | 5.17 |
| KCl | 0.35 |
| KH₂PO₄ | 0.0502 |
| MgSO₄•7H₂O | 0.0492 |
| CaCl₂•2H₂O | 0.294 |
| NaHCO₃ | 1.68 |
| HEPES | 0.953 |
| Glucose | 1.8 |
| Gentamicin Sulfate | 0.010 |
| Phenol Red | 0.010 |

The components of the kits provide for initial incubation of sperm in nutrient free synthetic HTF that does not contain a glycolytic energy source (e.g., glucose) or a gluconeogenesis substrate (e.g., pyruvate), and then later a glycolytic energy source or a gluconeogenesis substrate are added simultaneously or sequentially resulting in improved sperm function. Gluconeogenesis substrate means a non-carbohydrate carbon sources that is used in the process of gluconeogenesis. The gluconeogenesis substrate acts as substrate for the gluconeogenic pathway, further acting to facilitate gluconeogenesis. Gluconeogenesis substrate suitable for use in the kits of the present disclosure include, but are not limited to, pyruvate, lactate, succinate, citrate, fumarate, malate, aspartate, glycerol, acetyl CoA, isocitrate, alpha-ketoglutarate, succinyl-CoA, oxaloacetate; or a physiologically acceptable derivative, salt, ester, polymer or alpha-keto analogue of the gluconeogenesis substrate. Any gluconeogenic amino acid, or a physiologically acceptable derivative, salt, ester, or polymer, or alpha-keto analogue thereof is also suitable as a gluconeogenesis substrate. Non-limiting examples of gluconeogenic amino acids include alanine, arginine, asparagine, cystine, glutamine, glycine, histidine, hydroxyproline, methionine, proline, serine, threonine and valine. Non-limiting examples of pharmaceutically acceptable salts of pyruvate are lithium pyruvate, sodium pyruvate, potassium pyruvate, magnesium pyruvate, calcium pyruvate, and zinc pyruvate. In some embodiments, the pyruvate is sodium pyruvate. Non-limiting examples of salts of lactate include sodium lactate, potassium lactate, magnesium lactate, calcium lactate, zinc lactate, and manganese lactate. The pyruvate component of the kit can be substituted with any other gluconeogenesis substrate listed above.

Glycolytic energy source includes carbon sources for glycolysis. Non-limiting examples of glycolytic energy source include monosaccharides (such as fructose, glucose, galactose and mannose) and disaccharides (sucrose, lactose, maltose, and trehalose), as well as polysaccharides, galactose, oligosaccharides, polymers thereof. The glucose component of the kit can be substituted with any other glycolytic energy source listed above.

In some embodiments, the kits comprise additional components, for example, other components upstream and downstream of glycolysis such as NADH, NAD+, citrate, AMP, ADP, or a combination thereof are added in combination with at least the first energy source or the second energy source.

It is understood that physiologically acceptable means non-toxic to sperm, oocytes or embryos, and which additionally improves their function and survival during in vitro and in vivo handling and manipulation.

### Example 8 Kit for Density Gradient

The kit for density gradient is useful to separate motile sperm from the ejaculate, using density gradient centrifugation. It is composed of a density gradient reagent i.e., suspension of silanized silica in nutrient-free sHTF. The kit for density gradient separation includes more than one concentration of silanized silica, which is provided e.g., based on user preferences, particularly among technicians in IVF clinics. In addition, the nutrient-free sHTF component of kit for IVF and kit for IUI can be used to dilute density gradient reagent to allow users to customize the concentration of silanized silica.

The kit for density gradient also includes instructions for use, which will mirror current clinical practice with density gradients. In general, semen samples are applied to the surface of the density gradient and centrifuged. Motile sperm are collected from the pellet at the bottom of the tube with a pipette. The sample is then ready for washing.

The density gradient reagent lacks nutrients that will be added later from the kit for IVF or the kit for IUI during sperm washing.

The kit for density gradient is designed without nutrients for the convenience of users of the kit for IVF and the kit for IUI. Without a nutrient-free density gradient, technicians would have to wash the sperm more times to prepare it for the staged introduction of nutrients provided through the use of the kit for IVF and the kit for IUI, using additional time and resources. The density gradient reagent has a pH of approximately 7.4.

### Example 9 Kit for IVF

The kit for IVF is useful for washing sperm, isolating motile viable sperm by swim-up, and diluting the density gradient reagent. The kit prepares sperm for the fertilization step in IVF. The kit for IVF contains 3 reagents: nutrient-free sHTF and concentrated solutions of pyruvate and glucose (Tables 1-5). The kit also includes instructions for use. The instructions for use include instructions on conducting a washing step and also include instructions related to the timed sequential addition of pyruvate and glucose. The general use of the kit is explained below:

### Sperm Separation: Swim-Up

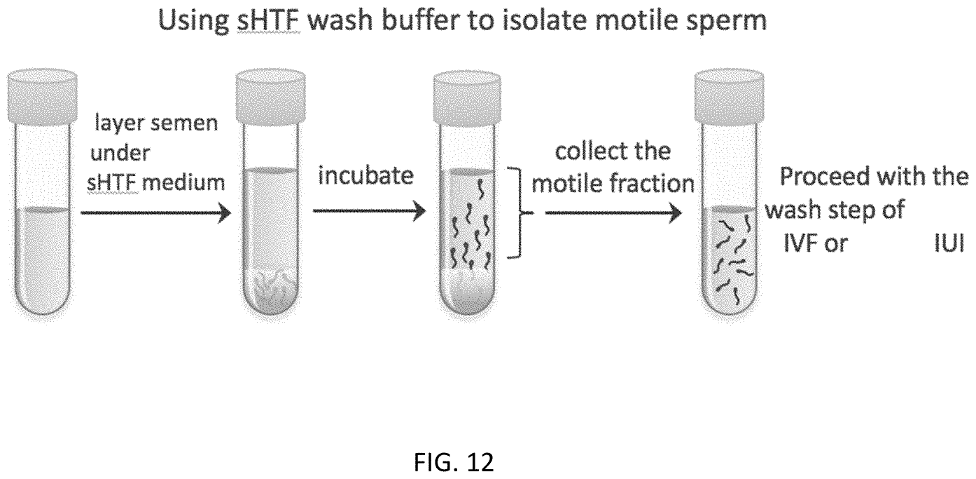

In most clinics, density gradient centrifugation is preferred over swim-up as a separation method. However, to accommodate user preferences, the nutrient-free sHTF reagent can be used for isolating motile viable sperm by swim-up (FIG. 12). The semen sample is first layered beneath a small volume of nutrient-free sHTF. Sperm is then given sufficient time to swim up into the nutrient-free sHTF. Finally, the top nutrient-free sHTF layer is collected and centrifuged.

### Sperm Separation: Diluting Density Gradient Reagent

When used to dilute density gradient reagent, nutrient-free sHTF is added to density gradient reagent to achieve the desired concentration. Preferred concentration of silanized silica in density gradient media can vary clinic to clinic. The kit for IVF allows users to customize the concentration of density gradient reagent to fit their needs.

### Washing Sperm

After sperm separation, the pelleted sperm is washed once in nutrient-free sHTF and incubated briefly. Glucose is added, and the sperm is incubated as detailed in the kit instructions. Then, pyruvate is added, and the sperm is incubated as detailed in the kit instructions before being centrifuged a final time. The sample is ready for resuspension in a fertilization medium for co-incubation with the oocyte.

The kit for IVF prepares sperm for the fertilization step in IVF. The kit for IVF includes three reagents—nutrient-free sHTF, glucose containing reagent, and pyruvate containing reagent—to allow for staged introduction of nutrients. Glucose and pyruvate are present as sequential additives rather than being pre-mixed. Table 2 and table 5 provide exemplary compositions of a reagent containing glucose. Table 2 and table 4 provide exemplary compositions of a reagent containing pyruvate.

### Example 10 Kit for IUI

The kit for IUI is used for washing sperm, isolating motile viable sperm by swim-up, diluting the density gradient reagent, and holding sperm for IUI procedure. The kit for IUI does not include glucose, but is otherwise identical to the kit for IVF. The instructions for use of the IVF and IUI kits is also generally identical, with the main difference being that instructions in the kit for IUI do not include the step of adding glucose. The instructions end with the preparation being ready for intrauterine insemination. Glucose is present in the uterus at concentrations at or above the final concentration of glucose in the kit for IVF's nutrient-free sHTF-glucose-pyruvate mixture. Therefore, sperm in the prepared sample is sufficiently exposed to glucose upon intrauterine injection to induce hyperactivation.

The kit for IUI includes two reagents—nutrient-free sHTF and pyruvate containing reagent—allowing for staging the introduction of pyruvate in vitro (included as a separate reagent) and glucose naturally (through in uterine exposure). In the kit for IUI glucose and pyruvate are initially absent from the media. Pyruvate is added after initial nutrient-free incubation. Glucose is not added ex vivo, but is provided upon intrauterine insemination of the sperm. This has the added benefit of synchronizing sperm hyperactivation triggered by exposure to pyruvate with the time of intrauterine injection. Table 2 and table 5 provide exemplary compositions of a reagent containing glucose. Table 2 and table 4 provide exemplary compositions of a reagent containing pyruvate.

### Example 11—Indications for Use

### Kit for Density Gradient

The kit for density gradient is useful to separate motile sperm from the ejaculate, using density gradient centrifugation. It is indicated to be used in conjunction with the kit for IVF or the kit for IUI.

### Kit for IVF

The kit for IVF is useful for washing sperm, isolating motile viable sperm by swim-up, and diluting the density gradient reagent in the kit for density gradient separation.

The kit prepares sperm for the fertilization step in IVF. When density gradient centrifugation is performed to separate sperm from the seminal fluid prior to preparing sperm using the kit for IVF, the kit for density gradient can be used.

### Kit for IUI

The kit for IUI is useful for washing sperm, isolating motile viable sperm by swim-up, diluting the density gradient reagent in kit for density gradient, and holding sperm for IUI procedure. When density gradient centrifugation is performed to separate sperm from the seminal fluid prior to preparing sperm using kit for IUI, the kit for density gradient separation can be used.

### Example 12—Improved Offspring Metabolic Fitness

Obesity is a growing worldwide public health concern because of its association with many human diseases, including type 2 diabetes, cardiovascular diseases, respiratory diseases, arthritis, and cancers. Most cases of obesity result from a mismatch in energy intake and energy expenditure combined with genetic pre-disposition. In addition to classic genetic inheritance of risk genes, epigenetics messages may be incorporated into the sperm. This example illustrates the effect of sperm subject to the methods provided by the invention on offspring metabolic fitness.

A diet-induced obesity model is used to test impact of starvation/rescue treatment of sperm from obese mice on fertility and offspring body mass. Male C57BL/6 mice are fed normal chow or a high fat diet. Sperm are collected and capacitated under control conditions (C-HTF, control) or following the starvation/rescue procedure (F-HTF, with staged reintroduction of glucose and pyruvate, test) described in Example 2. Sperm are analyzed for changes in motility total RNA (as well as small non-coding RNA, including micro RNA) content, and DNA, RNA, and protein methylation between control and test conditions. RNAseq is performed to evaluate changes in RNA, such as small non-coding RNA, including microRNA. Bisulfite sequencing is performed to evaluate changes in DNA methylation. In vitro fertilization is performed using sperm from control and test conditions, and the number of fertilized eggs, blastocyst formation, and live births is evaluated. Finally, RNA seq and bisulfite sequencing for DNA methylation is performed on 2-cell embryos from each experimental condition. Additionally, body mass of offspring from each condition is monitored for 12 months, as well as periodic blood chemistry analysis. Sperm subject to test conditions show reduced RNA levels (including micro RNA) and/or changes in DNA methylation compared to controls and corresponding pups show reduced obesity relative to controls.

Effects of nutrient deprivation and reintroduction on RNA content and methylation and acetylation of DNA, RNA and proteins will be assessed on sperm obtained from obese or overweight males. Sperm are isolated and exposed to control conditions or the starvation/rescue procedures (F-HTF with staged reintroduction of glucose and pyruvate, test). Samples will be analyzed for changes in RNA content, DNA, RNA and protein methylation and acetylation following starvation phase and reintroduction of each nutrient.

### Example 13 Exemplary Preservation Medium

### Samples and Methods

### Semen Samples and Sperm Preparation

A cohort of unselected donors supplied semen samples for this study. Samples were produced by masturbation into a sterile container and delivered to the laboratory within 1 hour of ejaculation. Fractionation of semen samples was achieved by density gradient centrifugation (Isolate, Irvine Scientific). See Tarchala S M, et al., 53rd Annual Meeting of the American Society for Reproductive Medicine, Cincinnati, Ohio; P-116, 1998. Following centrifugation for 20 minutes at 300×g, the seminal plasma fraction and the low density layer were removed, and the high-density fraction predominantly containing spermatozoa with a high percentage of viability, motility and normal morphology, was washed twice with the appropriate storage medium.

### Storage of Spermatozoa.

Electrolyte-free medium (EFM) was prepared as described by Riel et al. (Biol Reprod. 2011 September; 85(3):536-47). Test preservation Medium or test medium, consisted of 0.33 M glucose, 3% bovine serum albumin, 10 mM HEPES, 10 μg/ml gentamicin, pH adjusted to 6.8 in sterile cell culture quality water. The Refrigeration Medium (RM) consists of TYB (TES Tris and egg yolk buffer) with gentamicin (Sigma-Aldrich G1272). Washed sperm samples were resuspended in 0.5-1.0 mL medium (EFM, RM, or Test preservation Medium) and stored at 4° C. in a cooling incubator (Benchmark Scientific). For cryopreservation, samples were mixed (1:1 sample medium ratio) with freezing medium (TYB with glycerol and gentamicin) (FM, Irvine Scientific, Catalog #90128), slowly (0.5° C./minute) cooled at 4° C., then frozen and stored in the vapor phase of liquid nitrogen following the manufacturer's recommendations.

### Computer-Assisted Sperm-Motility Analysis (CASA)

Computer-assisted sperm-motility analysis (CASA) of semen and stored sperm samples was obtained with the CEROS II system from Hamilton Thorne following the protocol recommenced by Goodson et al. (Biol Reprod. 2017 Nov. 1; 97(5):698-708). The relative distribution of static and motile sperm, as well as the curvilinear velocity (VCL) was determined in at least 500 sperm. Before CASA, sperm were allowed to recover for 4 hours in conditions known to induce capacitation, modified human tubal fluid (mHTF) with 5 mg/mL human serum albumin, 25 mM sodium bicarbonate, pH 7.3. 6 μl of sperm suspensions were loaded into disposable 20 μm chamber slides (Leja Products SC 20-01-02-B) and videos were acquired for CASA (1 second, 60 frames per second).

Terminal Deoxynucleotidyl Transferase dUTP Nick End Labelling (TUNEL)

DNA fragmentation was measured using the APO-DIRECT kit (BD Biosciences 556381) following the protocol recommenced by Simon et al. (Hum Reprod. 2014 May; 29(5):904-17) on 3×10\hat{ }6 cells. The presence of free 3′-OH groups measured with the CytoFLEX flow cytometer (Beckman Coulter Life Sciences).

### Lipid Peroxidation Assay.

Peroxidative damage in sperm was assessed using the lipid peroxidation sensor BODIPY C11 (Thermo Fisher Scientific D3861) on 1×10\hat{ }6 cells. The shift of the fluorescence emission peak was measured with the CytoFLEX flow cytometer relative to positive controls (sample incubated with 80 uM ferrous sulfate) and negative controls (no dye).

### Data Processing

Flow cytometry raw data were processed with FlowJo v10 (FlowJo, LLC). CASA and flow cytometry data were imported in Prism 8 (GraphPad) for statistical analysis.

### Results

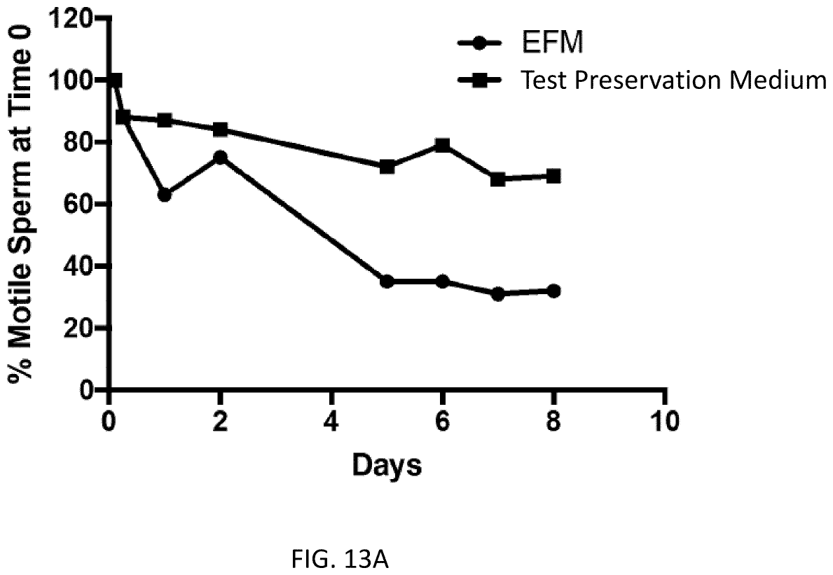

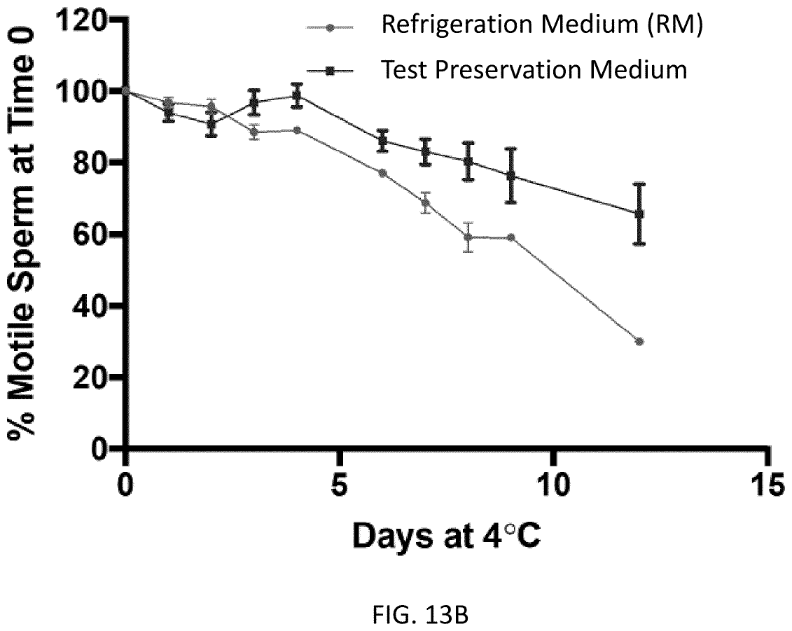

The motility of human sperm from multiple n donors was assessed using CASA during short term storage at 4° C. in electrolyte free medium (EFM), refrigeration medium (RM), or Test preservation Medium. Up to 80% initial sperm motility was preserved after 7 days of storage in Test preservation Medium, which represented a significant improvement over storage in EFM (FIG. 13A, 75% in Test Medium vs. 35% in EFM) and storage in RM (FIG. 13B, 80% in Test Medium vs. 60% in EFM). Up to 50% initial sperm motility was preserved after 14 days storage in Test preservation Medium.

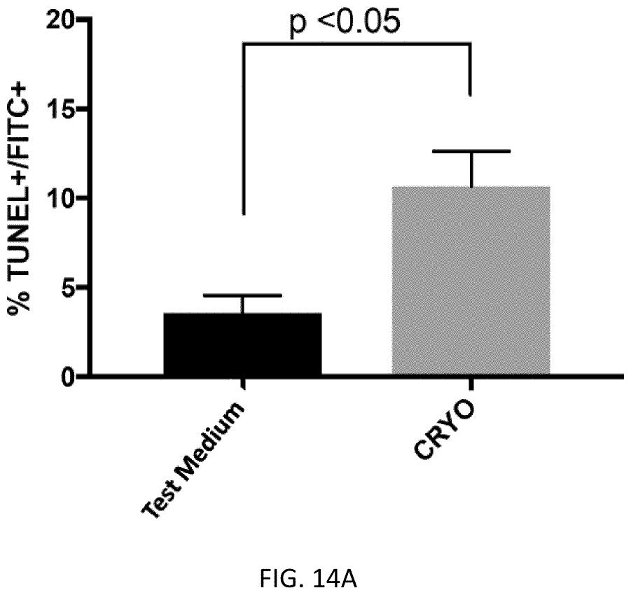

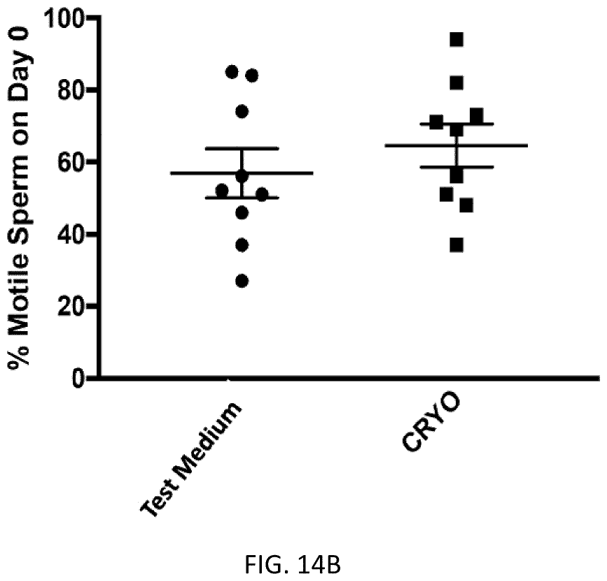

Short term storage in Test preservation Medium compares favorably with cryopreservation by preventing sperm DNA damage. Fresh human sperm samples were stored for 7 days in Test Medium at 4° C., or diluted in Irvine freezing medium and frozen in liquid nitrogen. After 7 days, the motility parameters (assessed by CASA) of Test Medium-preserved sperm and cryopreserved sperm were not significantly different (FIG. 14A). However, TUNEL analyses revealed that DNA fragmentation was significantly lower in Test Medium-preserved sperm than in cryopreserved and thawed sperm (FIG. 14B), suggesting that storage in Test Medium (test preservation medium) at 4° C. is less detrimental to sperm than cryopreservation for short-term storage.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

All publications, patents, and patent applications mentioned in this specification are herein incorporated by reference in their entirety for all purposes, to the same extent as if each individual publication, patent, or patent application was specifically and individually indicated to be incorporated by reference. For example, all publications and patents mentioned herein are incorporated herein by reference in their entirety for the purpose of describing and disclosing the kits, compositions, and methodologies that are described in the publications, which might be used in connection with the methods, kits, and compositions described herein. The documents discussed herein are provided solely for their disclosure prior to the filing date of the present application. Nothing herein is to be construed as an admission that the inventors described herein are not entitled to antedate such disclosure by virtue of prior invention or for any other reason. Where any conflict exists between a document incorporated by reference and the present disclosure, this disclosure will control.

Headings used in this application are for convenience only and do not affect the interpretation of this application. Headings should not be used to limit the invention in any way. For example, methods described under the heading sperm function, should not be limited such that the methods cannot be performed as described under the heading sperm motility, or with the reagents disclosed under the heading preservation media. Rather, it is intended that the methods and reagents disclosed and described under the various headings are wholly interchangeable and can be performed in any combination such that one of skill in the art would be able to select the disclosure from any portion of the description of the invention herein to combine with any other portion of the description of the invention herein.

Preferred features of each of the aspects provided by the invention (e.g., media, compositions, preparations, and methods) are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

What is claimed is:

1. A method of enhancing sperm function, the method comprising:
(a) incubating a human sperm in a composition for a time suitable to generate a potentiated human sperm, wherein the composition comprises:
(i) about 1, 2, 4, 5, 6, 7, 8, 9, 10, 11, 12, or 15 mM HEPES;
(ii) one or more salts; and
(iii) 18, 20, or 22 mM NaHCO₃;
wherein the composition is substantially pyruvate-free and substantially glucose-free; and
(b) providing the potentiated human sperm with an effective amount of an energy source, wherein the effective amount is an amount sufficient to induce increased sperm function.

2. The method of claim 1, wherein the incubating of (a) is for at least about 1 hour.

3. The method of claim 1, wherein providing the potentiated human sperm with the effective amount of an energy source comprises incubating the sperm with the energy source for at least about 30 minutes.

4. The method of claim 1, wherein providing the potentiated human sperm with the effective amount of an energy source comprises incubating the sperm with the energy source for at least about 1 hour.

5. The method of claim 1, wherein the composition comprises about 10 mM HEPES.

6. The method of claim 1, wherein the composition comprises about 4 mM HEPES.

7. The method of claim 1, wherein the NaHCO₃ is at a concentration of about 20 mM.

8. The method of claim 1, wherein the energy source is a gluconeogenesis substrate.

9. The method of 8, wherein the gluconeogenesis substrate is pyruvate.

10. The method of claim 1, wherein the energy source is a glycolytic energy source.

11. The method of claim 10, wherein the glycolytic energy source is glucose.

12. The method of claim 1, which further comprises:
subsequently providing the mammalian sperm with an effective amount of a second energy source, wherein the effective amount is an amount sufficient to induce increased sperm function.

13. The method of claim 12, wherein the energy source and the second energy source are provided sequentially or simultaneously.

14. The method of claim 12, wherein the first energy source is a gluconeogenesis substrate and the second energy source is a glycolytic energy source.

15. The method of claim 13, wherein in the glycolytic energy source is glucose.

16. The method of claim 12, wherein the first energy source is pyruvate and the second energy source is glucose.

17. The method of claim 12, wherein providing the sperm with the effective amount of the second energy source comprises incubating the sperm with the second energy source for at least about 15 minutes.

18. The method of claim 1, which further comprises artificial insemination in the vagina or intrauterine insemination (IUI) of the human sperm.

19. The method of claim 12, which further comprises using the human sperm to perform in vitro fertilization (IVF).

20. The method of claim 1, wherein the human sperm is from an oligospermic subject or a subfertile subject.
